(19) United States
(12) Patent Application Publication
(10) Pub. No.: US 2024/0247250 A1
(43) Pub. Date: Jul. 25, 2024

(54) COMPOSITIONS AND METHODS FOR MODIFYING DNA

(71) Applicant: The Regents of the University of California, Oakland, CA (US)

(72) Inventors: Ember M. Tota, La Jolla, CA (US); Neal Devaraj, La Jolla, CA (US)

(21) Appl. No.: 18/563,336

(22) PCT Filed: May 24, 2022
(86) PCT No.: PCT/US2022/030760

§ 371 Date: Nov. 21, 2023

Related U.S. Application Data
(60) Provisional application No. 63/192,456, filed on May 24, 2021.

(51) Int. Cl. C12N 15/10; C07D 487/04; C12N 9/10

(52) U.S. Cl. CPC C12N 15/102; C07D 487/04; C12N 9/1077; C12Y 204/02029

(57) ABSTRACT

Provided herein are, inter alia, compositions and methods for modifying a DNA molecule. The methods include contacting a DNA molecule with a transglycosylase enzyme and a Pre-queuosinel (preQi) analog or derivative, wherein a guanine nucleobase within a hairpin structure in the DNA molecule is exchanged for the preQi analog or derivative.

### CROSS-REFERENCE TO RELATED APPLICATIONS

This application claims priority to U.S. Provisional Application No. 63/192,456, filed May 24, 2021, which is hereby incorporated by reference in its entirety and for all purposes.

### STATEMENT AS TO RIGHTS TO INVENTIONS MADE UNDER FEDERALLY SPONSORED RESEARCH AND DEVELOPMENT

This invention was made with government support under GM123285 awarded by the National Institutes of Health. The government has certain rights in the invention.

### BACKGROUND

While harnessing the programmable power of nucleic acids is no new revelation for science, new innovative applications that realize this power have been crucial to scientific advancements of late. These innovative strategies often rely heavily on nucleic acid modifications used for visualization, immobilization, conjugation, and affinity interactions, among others. Modification strategies include the site-specific insertion of synthetic nucleotides bearing a functional group of interest, non-site-specific chemical modification of nucleobases or the sugar backbone, and a limited set of enzymatic modification techniques. For many technical applications, precision is the key to its success, and it is necessary to have the means to carry out an efficient, site-specific, modification of the nucleic acid substrate. While a variety of site-specific enzymatic RNA modification strategies have been well established, the same is not true for DNA modifications, particularly single stranded DNA (ssDNA). Currently enzymatic modification of ssDNA is limited to 3′ insertion of modified nucleobases and the 5′ insertion of modified phosphate groups. Consequently, there is a need for higher precision methods and compositions for enzymatic modification of ssDNA. Provided herein are, inter alia, solutions to these and other problems in the art.

### BRIEF SUMMARY

In an aspect is provided a method of modifying a DNA molecule, the method including contacting the DNA molecule with a tRNA-guanine transglycosylase (TGT) enzyme and a PreQ1 analog, wherein the DNA molecule includes a guanine nucleobase within a loop portion of a hairpin in the DNA molecule, and wherein the PreQ1 analog has the formula:

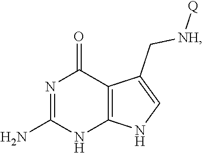

wherein Q is not hydrogen.

In another aspect is provided a method of substituting a guanine nucleobase within a DNA molecule with a PreQ1 analog, the method including contacting the DNA molecule and PreQ1 analog with a tRNA-guanine transglycosylase (TGT) enzyme, wherein the guanine nucleobase is within a loop portion of a hairpin in the DNA molecule, and wherein the PreQ1 analog has the formula:

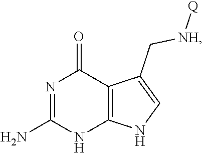

wherein Q is not hydrogen.

In another aspect is provided a method of modifying a DNA molecule, the method including contacting the DNA molecule with a tRNA-guanine transglycosylase (TGT) enzyme and a PreQ1 derivative, wherein the DNA molecule includes a guanine nucleobase within a loop portion of a hairpin in the DNA molecule, and wherein the PreQ1 derivative has the formula:

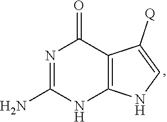

wherein Q is not hydrogen.

In an aspect is provided a method of substituting a guanine nucleobase within a DNA molecule with a PreQ1 derivative, the method including contacting the DNA molecule and PreQ1 derivative with a tRNA-guanine transglycosylase (TGT) enzyme, wherein the guanine nucleobase is within a loop portion of a hairpin in the DNA molecule, and

wherein the PreQ1 derivative has the formula:

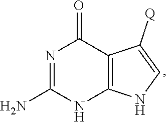

wherein Q is not hydrogen.

In an aspect is provided a DNA compound having the formula:

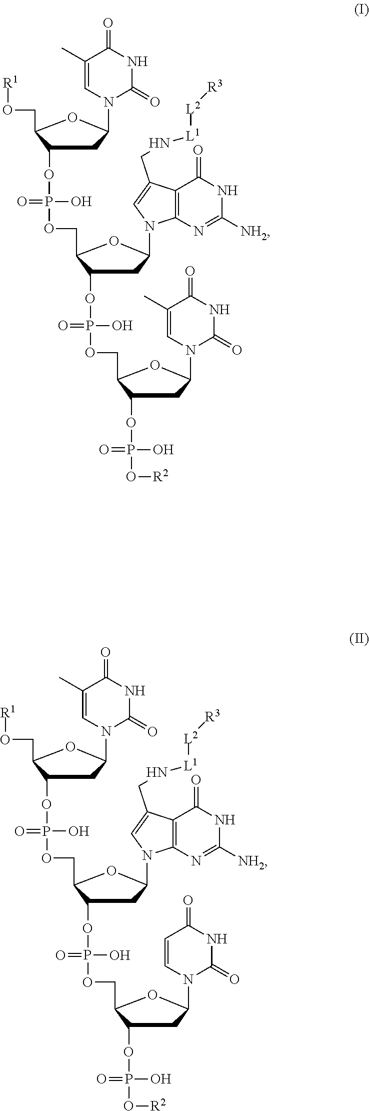

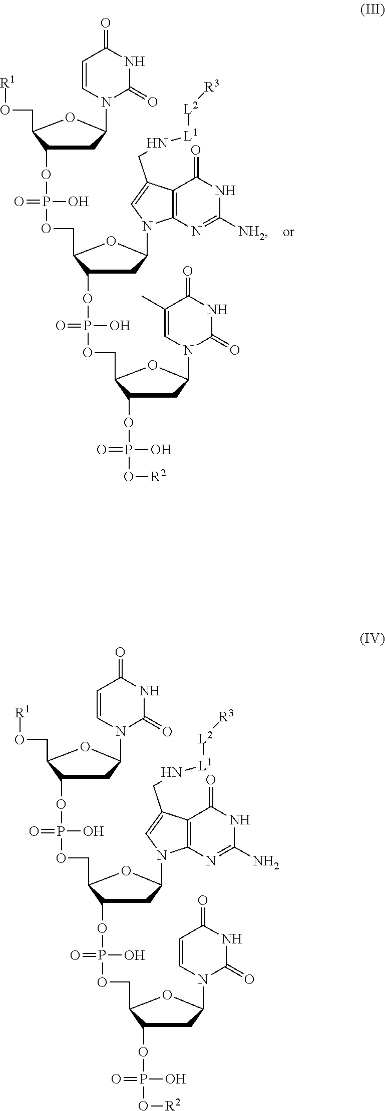

In another aspect is provided a DNA compound having the formula:

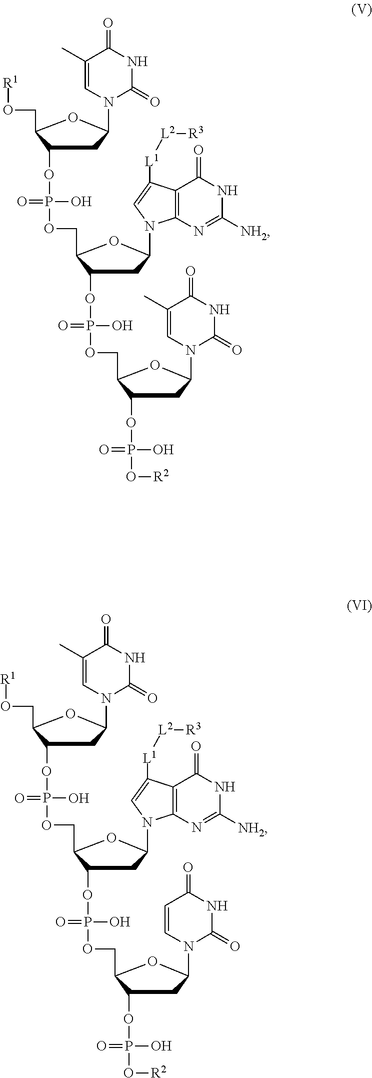

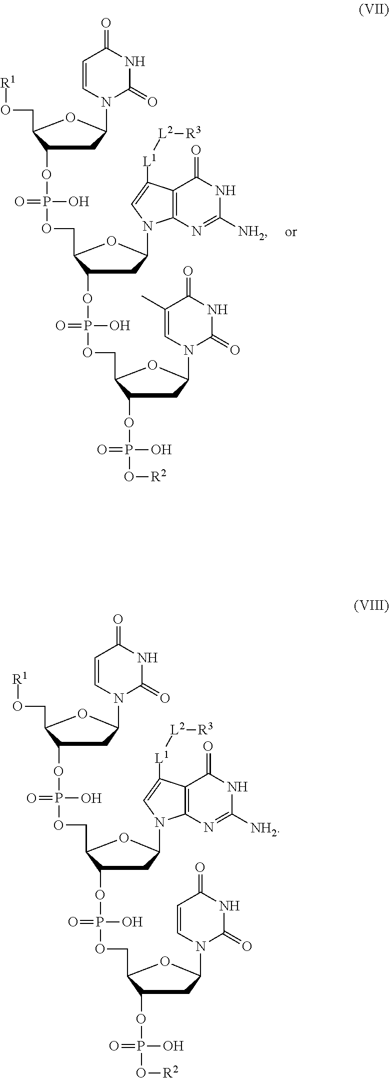

In another aspect a cell including the DNA compound provided herein including embodiments thereof is provided.

In an aspect is provided a method of modifying DNA, the method including: contacting a DNA molecule with a transglycosylase and a nucleoside derivative under conditions suitable to exchange at least one guanine nucleobase of the DNA for the nucleoside derivative, the DNA molecule comprising a hairpin comprising a loop sequence of 5′ YYGYYYY 3′ (SEQ ID NO: 103). In aspects, the hairpin loop is 5′ YTGTYYY 3′ (SEQ ID NO:104), 5′ YTGTCCY 3′ (SEQ ID NO:105), 5′ YTGTYCC 3′ (SEQ ID NO:106), 5′ YUGUYYY 3′ (SEQ ID NO:107), 5′ YUGTYYY 3′ (SEQ ID NO:108) or 5′ YTGUYYY 3′ (SEQ ID NO:9).

In an aspect is provided a method substituting a guanine with a PreQ1 analog within a DNA molecule, including (i) contacting a PreQ1 analog with an DNA molecule in the presence of a transglycosylase; and (ii) allowing the transglycosylase to substitute a guanine moiety from a guanine within the DNA sequence with the PreQ1 analog thereby forming a modified DNA molecule.

### BRIEF DESCRIPTION OF THE DRAWINGS

FIG. 1 is a schematic showing DNA-TAG insertion of a modified PreQ1 (e.g. PreQ1 analog, PreQ1 derivative) into a DNA molecule (SEQ ID NO:98).

FIGS. 2A-2C show TGT modification of modified DNA and RNA substrates. FIG. 2A. A reaction scheme showing insertion of PreQ1-Biotin. FIG. 2B. Thymine and Uracil nucleobase structures. FIG. 2C. TGT can modify RNA hairpin structures including T, and DNA hairpin structures including U, as illustrated in the representative images of gels showing a change in molecular weight of the modified DNA and RNA hairpins (bottom panel). Example sequences used in this study include SEQ ID NO:1, 2, 3, 4, 1, and 5 (top panel, left to right).

FIGS. 3A-3B show the steric limitations of DNA labeling. FIG. 3A. A representative image of a gel illustrating TGT modification of DNA molecules using analogs of various cognate anticodon loops. Sequences used in this study include 6, 7, 8, and 9 (top panel, right to left). FIG. 3B. TGT modification of anticodon loop analogs Sequences used in this study include SEQ ID NO: 10, 11, 12, 8, 13, 14, 9, 15, 16, 6, 17, 18, 7, 99, 100, and 101 (left panel, top to bottom).

FIGS. 4A-4C show assessment of TGT activity with various loop and stem sequences in the DNA molecule substrate. Representative gels showing modification of DNA sequences including HIS stem with various loops. FIG. 4A. The cononical sequences are shown above the representative image of the gels; SEQ ID NO:19 (top panel); SEQ ID NO:26 (bottom panel). TGT prefers loops with smaller pyrimidine bases, specifically those with the sequences YTGTCCY (SEQ ID NO:105) or YTGTYCC (SEQ ID NO:106) where Y is C or T. Loop from hairpin 130 is used to test different stems. Sequences used for this study include SEQ ID NO:20-25, as listed from top to bottom (top panel), and SEQ ID NO:27, 28, 18, 29-31 (bottom panel). FIG. 4B. The cononical sequences are shown above the representative image of the gels; SEQ ID NO:19 (top panel); SEQ ID NO:26 (bottom panel). Sequences used for this study include SEQ ID NO:32-37, as listed from top to bottom (top panel), and SEQ ID NO:38-43 (bottom panel). FIG. 4C. The cononical sequences are shown above the representative image of the gels; SEQ ID NO:19 (top panel); SEQ ID NO:26 (bottom panel). Sequences used for this study include SEQ ID NO:44-49, as listed from top to bottom (top panel), and SEQ ID NO:50 and 51 (bottom panel).

FIGS. 4D-4G. show that TGT can tolerate a variety of stems bearing TTGTCCT loop. Representative images of gels showing modification of DNA sequences with various stem sequences and the TTGTCCT loop. FIG. 4D. Sequences used for this experiment include SEQ ID NO:52-57 (left panel, top to bottom) and SEQ ID NO:58, 53, 59, 60, 61, and 62 (right panel, top to bottom). FIG. 4E. Sequences used for this experiment include SEQ ID NO:63-65, 21, 66, and 67 (left panel, top to bottom) and SEQ ID NO:68-73 (right panel, top to bottom). FIG. 4F. Sequences used for this experiment include SEQ ID NO:74, 73, 75-78 (left panel, top to bottom) and SEQ ID NO:79, 80, 80, 81-83 (right panel, top to bottom). FIG. 4G. Sequences used for this experiment include SEQ ID NO:84-86, 83, 87 and 88 (left panel, top to bottom) and SEQ ID NO:89 and 90 (right panel, top to bottom).

FIG. 5 shows deconvoluted mass spectrometry and LC trace confirm PreQ1-biotin labeling of hairpin 176.

FIGS. 6A-6C show that TGT can insert a variety of labels into the ssDNA hairpin at various positions. FIG. 6A. TGT can insert a variety of labels into the discovered DNA hairpin sequence with high efficiency. FIG. 6B. TAG2 DNA hairpin can be modified internally. Sequences used for this study include SEQ ID NO:91-93 (top to bottom). FIG. 6C. TAG2 DNA hairpin can be modified at either end of a DNA sequence of interest. Sequences used for this study include SEQ ID NO:94-97.

FIG. 7 shows DNA-TAG labeled probe sets co-localize with commercially available RNA FISH probe sets.

FIGS. 8A-8B show dose dependent U6 RNA detection using SiR-PreQ1 labeled U6 antisense probe. FIG. 8A. A representative image of a blot illustrating in vitro transcribed U6 RNA and FIG. 8B. A representative image of a blot illustrating U2OS cellular RNA.

### DETAILED DESCRIPTION

Before the present invention is further described, it is to be understood that this invention is not strictly limited to particular embodiments described, as such may of course vary. It is also to be understood that the terminology used herein is for the purpose of describing particular embodiments only, and is not intended to be limiting, since the scope of the present invention will be limited only by the claims.

It must be noted that as used herein and in the appended claims, the singular forms “a,” “an,” and “the” include plural referents unless the context clearly dictates otherwise. It should further be understood that as used herein, the term “a” entity or “an” entity refers to one or more of that entity. For example, a nucleic acid molecule refers to one or more nucleic acid molecules. As such, the terms “a”. “an”, “one or more” and “at least one” can be used interchangeably. Similarly, the terms “comprising”, “including” and “having” can be used interchangeably.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed. In addition, all sub-combinations are also specifically embraced by the present invention and are disclosed herein just as if each and every such sub-combination was individually and explicitly disclosed herein.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono-, or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by,
—CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quatemized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to:
—CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). In embodiments, the heteroalkyl is fully saturated. In embodiments, the heteroalkyl is monounsaturated. In embodiments, the heteroalkyl is polyunsaturated. The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as
C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkene. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quatemized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzooxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocyclic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

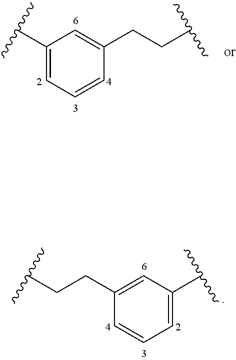 [SMILES: *CCc1cccc(*)c1 ; *CCc1cccc(*)c1]

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen,
—SiR′R″R′, —OC(O)R′, —C(O)R′, —CO₂R′, —CONR′R″, —OC(O)NR′R″, —NR″C(O)R′, —NR′C(O)NR″R″, —NR″C(O)₂R′, —NRC(NR′R″R″)═NR″″, —NRC(NR′R″)═NR″′, —S(O)R′,
—S(O)₂R′, —S(O)₂NR′R″, —NRSO₂R′, —NR′NR″R″′, —ONR′R″, —NR′C(O)NR″NR″′R″″, —CN, —NO₂, —NR′SO₂R″, —NR′C(O)R″, —NR′C(O)OR″, —NR′OR″, —N₃, in a number ranging from zero to (2m′+1), where m′ is the total number of carbon atoms in such radical. R, R′, R″, R″′, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R″′, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen,
—SiR′R″R′, —OC(O)R′, —C(O)R′, —CO₂R′, —CONR′R″, —OC(O)NR′R″, —NR″C(O)R′, —NR′C(O)NR″R″, —NR″C(O)₂R′, —NRC(NR′R″R″′)═NR″″, —NRC(NR′R″)═NR″′, —S(O)R′,
—S(O)₂R′, —S(O)₂NR′R″, —NRSO₂R′, —NR′NR″R″′, —ONR′R″, —NR′C(O)NR″NR″′R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″,
NR′C(O)—OR″, —NR′OR″, in a number ranging from zero to the total number of open valences on the aromatic ring system; and where R′, R″, R″′, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R″′, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—,
CRR′—, or a single bond, and q is an integer of from 0 to 3. Alternatively, two of the substituents on adjacent atoms of the aryl or heteroaryl ring may optionally be replaced with a substituent of the formula -A—(CH₂)ᵣ—B—, wherein A and B are independently —CRR′—, —O—, —NR—, —S—, —S(O)—,
—S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R″′)_{d}—, where s and d are independently integers of from 0 to 3, and X′ is —O—, —NR′—, —S—, —S(O)—, —S(O)₂—, or —S(O)₂NR′—. The substituents R, R′, R″, and R″′ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), selenium (Se), phosphorus (P), and silicon (Si).

A “substituent group,” as used herein, means a group selected from the following moieties:
(A) oxo,
halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂,
—OCHF₂, —OCH₂Cl, —OCH₂Br, —OCH₂I, —OCH₂F, —CN, —OH, —NH₂, —COOH, —CONH₂,
—NO₂, —SH, —SO₃H, —OSO₃H, —SO₂NH₂, —NHNH₂, —ONH₂, —NHC(O)NHNH₂,
—NHC(O)NH₂, —NHSO₂H, —NHC(O)H, —NHC(O)OH, —NHOH, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₂₀, C₁-C₁₂, C₁-C₈, C₁-C₆, C₁-C₄, or C₁-C₂), heteroalkyl (e.g., 2 to 20 membered, 2 to 12 membered, 2 to 8 membered, 2 to 6 membered, 4 to 6 membered, 2 to 3 membered, or 4 to 5 membered), cycloalkyl (e.g., C₃-C₁₀, C₃-C₈, C₃-C₆, C₄-C₆, or C₅-C₆), heterocycloalkyl (e.g., 3 to 10 membered, 3 to 8 membered, 3 to 6 membered, 4 to 6 membered, 4 to 5 membered, or 5 to 6 membered), aryl (e.g., C₆-C₁₂, C₆-C₁₀, or phenyl), or heteroaryl (e.g., 5 to 12 membered, 5 to 10 membered, 5 to 9 membered, or 5 to 6 membered), substituted with at least one substituent selected from:
(i) oxo,
halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂,
—OCHF₂, —OCH₂Cl, —OCH₂Br, —OCH₂I, —OCH₂F, —CN, —OH, —NH₂, —COOH, —CONH₂,
—NO₂, —SH, —SO₃H, —OSO₃H, —SO₂NH₂, —NHNH₂, —ONH₂, —NHC(O)NHNH₂,
—NHC(O)NH₂, —NHSO₂H, —NHC(O)H, —NHC(O)OH, —NHOH, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(ii) alkyl (e.g., C₁-C₂₀, C₁-C₁₂, C₁-C₈, C₁-C₆, C₁-C₄, or C₁-C₂), heteroalkyl (e.g., 2 to 20 membered, 2 to 12 membered, 2 to 8 membered, 2 to 6 membered, 4 to 6 membered, 2 to 3 membered, or 4 to 5 membered), cycloalkyl (e.g., C₃-C₁₀, C₃-C₈, C₃-C₆, C₄-C₆, or C₅-C₆), heterocycloalkyl (e.g., 3 to 10 membered, 3 to 8 membered, 3 to 6 membered, 4 to 6 membered, 4 to 5 membered, or 5 to 6 membered), aryl (e.g., C₆-C₁₂, C₆-C₁₀, or phenyl), or heteroaryl (e.g., 5 to 12 membered, 5 to 10 membered, 5 to 9 membered, or 5 to 6 membered), substituted with at least one substituent selected from:
(a) oxo,
halogen, —CCl₃, —CBr₃, —CF₃, —CI₃, —CHCl₂, —CHBr₂, —CHF₂, —CHI₂, —CH₂Cl,
—CH₂Br, —CH₂F, —CH₂I, —OCCl₃, —OCF₃, —OCBr₃, —OCI₃, —OCHCl₂, —OCHB r₂,
—OCHI₂, —OCHF₂, —OCH₂Cl, —OCH₂Br, —OCH₂I, —OCH₂F, —CN, —OH, —NH₂, —COOH,
—CONH₂, —NO₂, —SH, —SO₃H, —OSO₃H, —SO₂NH₂, —NHNH₂, —ONH₂, —NHC(O)NHNH₂,
—NHC(O)NH₂, —NHSO₂H, —NHC(O)H, —NHC(O)OH, —NHOH, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(b) alkyl (e.g., C₁-C₂₀, C₁-C₁₂, C₁-C₈, C₁-C₆, C₁-C₄, or C₁-C₂), heteroalkyl (e.g., 2 to 20 membered, 2 to 12 membered, 2 to 8 membered, 2 to 6 membered, 4 to 6 membered, 2 to 3 membered, or 4 to 5 membered), cycloalkyl (e.g., C₃-C₁₀, C₃-C₈, C₃-C₆, C₄-C₆, or C₅-C₆), heterocycloalkyl (e.g., 3 to 10 membered, 3 to 8 membered, 3 to 6 membered, 4 to 6 membered, 4 to 5 membered, or 5 to 6 membered), aryl (e.g., C₆-C₁₂, C₆-C₁₀, or phenyl), or heteroaryl (e.g., 5 to 12 membered, 5 to 10 membered, 5 to 9 membered, or 5 to 6 membered), substituted with at least one substituent selected from: oxo,
halogen, —CCl₃, —CBr₃, —CF₃, —CI₃, —CHCl₂, —CHBr₂, —CHF₂. CHI₂, —CH₂Cl, —CH₂Br, —CH₂F, —CH₂I, —OCCl₃, —OCF₃, —OCBr₃, —OCI₃, —O CHCl₂,
—OCHBr₂, —OCHI₂, —OCHF₂, —OCH₂Cl, —OCH₂Br, —OCH₂I, —OCH₂F, —CN, —OH,
—NH₂, —COOH, —CONH₂, —NO₂, —SH, —SO₃H, —OSO₃H, —SO₂NH₂, —NHNH₂, —ONH₂, —NHC(O)NHNH₂,
—NHC(O)NH₂, —NHSO₂H, —NHC(O)H, —NHC(O)OH, —NHOH, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and/or each substituted or unsubstituted heteroaryl is a substituted or unsubstituted 5 to 6 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted phenylene, and/or each substituted or unsubstituted heteroarylene is a substituted or unsubstituted 5 to 6 membered heteroarylene. In some embodiments, the compound is a chemical species set forth in the application (e.g., Examples section, figures, or tables below).

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on an R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R^{100.1}. As a further example, R^{1A} may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1}. As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1}, L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1}, respectively. In turn, each first substituent group (e.g., R^{1.1}, R^{2.1}, R^{3.1}, R^{4.1}, R^{5.1} . . . R^{100.1}; R^{1A.1}, R^{2A.1}, R^{3A.1}, R^{4A.1}, R^{5A.1} . . . R^{100A.1}; R^{L1.1}, R^{L2.1}, R^{L3.1}, R^{L4.1}, R^{L5.1} . . . R^{L100.1}) may be further substituted with one or more second substituent groups (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}) may be further substituted with one or more third substituent groups (e.g., R^{1.3}, R^{2.3}, R^{3.3}, R^{4.3}, R^{5.3} . . . R^{100.3}; R^{1A.3}, R^{2A.3}, R^{3A.3}, R^{4A.3}, R^{5A.3} . . . R^{100A.3}; R^{L1.3}, R^{L2.3}, R^{L3.3}, R^{L4.3}, R^{L5.3} . . . R^{L100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R″ may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g., when R^{WW.1} is R^{WW.2}-substituted or unsubstituted alkyl, examples of groups so formed include but are not limited to itself optionally substituted by 1 or more R^{WW.2} which R^{WW.2} is optionally substituted by one or more R^{WW.3}. By way of example when the R^{WW} group is phenyl substituted by R^{WW.1}, which is methyl, the methyl group may be further substituted to form groups including but not limited to:

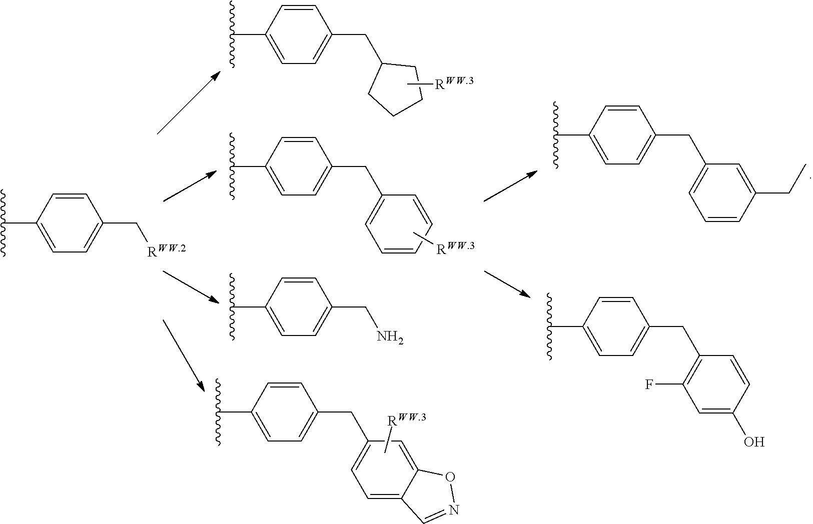

R^{WW.1} is independently oxo,
halogen, —CX^{WW.1}₃, —CHX^{WW.1}₂, —CH₂X^{WW.1}, —OCX^{WW.1}₃,
—OCH₂X^{WW.1}, —OCHX^{WW.1}₂, —CN, —OH, —NH₂, —COOH, —CONH₂, —NO₂, —SH, —SO₃H, —OSO₃H,
—SO₂NH₂, —NHNH₂, —ONH₂, —NHC(O)NHNH₂,
—NHC(O)NH₂, —NHSO₂H, —NHC(O)H,
—NHC(O)OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo, halogen,
—CX^{WW.1}₃, —CHX^{WW.1}₂, —CH₂X^{WW.1}, —OCX^{WW.1}₃, —OCH₂X^{WW.1}, —OCHX^{WW.1}₂, —CN, —O H, —NH₂,
—COOH, —CONH₂, —NO₂, —SH, —SO₃H, —OSO₃H, —SO₂NH₂, —NHNH₂, —ONH₂, —NHC(O)NHNH₂,
—NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo,
halogen, —CX^{WW.2}₃, —CHX^{WW.2}₂, —CH₂X^{WW.2}, —OCX^{WW.2}₃,
—OCH₂X^{WW.2}, —OCHX^{WW.2}₂, —CN, —OH, —NH₂, —COOH, —CONH₂, —NO₂, —SH, —SO₃H, —OSO₃H,
—SO₂NH₂, —NHNH₂, —ONH₂, —NHC(O)NHNH₂,
—NHC(O)NH₂, —NHSO₂H, —NHC(O)H,
—NHC(O)OH, —NHOH, —N₃, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen,
—CX^{WW.2}₃, —CHX^{WW.2}₂, —CH₂X^{WW.2}, —OCX^{WW.2}₃, —OCH₂X^{WW.2}, —OCHX^{WW.2}₂, —CN, —O H, —NH₂,
—COOH, —CONH₂, —NO₂, —SH, —SO₃H, —OSO₃H, —SO₂NH₂, —NHNH₂, —ONH₂, —NHC(O)NHNH₂,
—NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered. 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo,
halogen, —CX^{WW.3}₃, —CHX^{WW.3}₂, —CH₂X^{WW.3}, —OCX^{WW.3}₃,
—OCH₂X^{WW.3}, —OCHX^{WW.3}₂, —CN, —OH, —NH₂, —COOH, —CONH₂, —NO₂, —SH, —SO₃H, —OSO₃H,
—SO₂NH₂, —NHNH₂, —ONH₂, —NHC(O)NHNH₂,
—NHC(O)NH₂, —NHSO₂H, —NHC(O)H,
—NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g., substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2} and each second substituent group, R^{WW.2}, may be unsubstituted or independently substituted with one or more third substituent groups, referred to herein as R^{WW.3}; and each third substituent group, R^{WW.3}, is unsubstituted. Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100A.1}, R^{WW.2} is R^{100A.2}, and R^{WW.3} is R^{100A.3}. Alternatively, in embodiments where R^{100A} and R^{100B} are optionally joined together to form an openly substituted ring, R^{WW.1} is R^{100B.1}, R^{WW.2} is R^{100B.2}, and R^{WW.3} is R^{100B.3}. R^{WW.1}, R^{WW.2} and R^{WW.3} in this paragraph are as defined in the preceding paragraphs.

R^{LWW.1} is independently oxo,
halogen, —CX^{LWW.1}₃, —CHX^{LWW.1}₂, —CH₂X^{LWW.1},
—OCX^{LWW.1}₃, —OCH₂X^{LWW.1}, —OCHX^{LWW.1}₂, —CN, —OH, —NH₂, —COOH, —CONH₂, —NO₂, —SH,
—SO₃H, —OSO₃H, —SO₂NH₂, —NHNH₂, —ONH₂, —NHC(O)NHNH₂,
—NHC(O)NH₂, —NHSO₂H,
—NHC(O)H, —NHC(O)OH, —NHOH, —N₃, R^{LWW.2}-substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo,
halogen, —CX^{LWW.1}₃, —CHX^{LWW.1}₂—CH₂X^{LWW.1}, —OCX^{LWW.1}₃, —OCH₂X^{LWW.1}, —OCHX^{LWW.1}₂
—CN, —OH, —NH₂, —COOH, —CONH₂, —NO₂, —SH, —SO₃H, —OSO₃H, —SO₂NH₂, —NHNH₂, —ONH₂, —NHC(O)NHNH₂,
—NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered. 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo,
halogen, —CX^{LWW.2}₃, —CHX^{LWW.2}₂, —CH₂X^{LWW.2},
—OCX^{LWW.2}₃, —OCH₂X^{LWW.2}, —OCHX^{LWW.2}₂, —CN, —OH, —NH₂, —COOH, —CONH₂, —NO₂, —SH,
—SO₃H, —OSO₃H, —SO₂NH₂, —NHNH₂, —ONH₂, —NHC(O)NHNH₂,
—NHC(O)NH₂, —NHSO₂H,
—NHC(O)H, —NHC(O)OH, —NHOH, —N₃, R^{LWW.3}-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo,
halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂,
—CN, —OH, —NH₂, —COOH, —CONH₂, —NO₂, —SH, —SO₃H, —OSO₃H, —SO₂NH₂, —NHNH₂, —ONH₂, —NHC(O)NHNH₂,
—NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered. 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo,
halogen, —CX^{LWW.3}₃, —CHX^{LWW.3}₂, —CH₂X^{LWW.3},
—OCX^{LWW.3}₃, —OCH₂X^{LWW.3}, —OCHX^{LWW.3}₂, —CN, —OH, —NH₂, —COOH, —CONH₂, —NO₂, —SH,
—SO₃H, —OSO₃H, —SO₂NH₂, —NHNH₂, —ONH₂, —NHC(O)NHNH₂,
—NHC(O)NH₂, —NHSO₂H,
—NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW}₃, —CHX^{WW}₂, —CH₂X^{WW},
—OCX^{WW}₃, —OCH₂X^{WW}, —OCHX^{WW}₂, —CN, —OH, —NH₂, —COOH, —CONH₂, —NO₂, —SH, —SO₃H,
—OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R^{WW.1}-substituted or unsubstituted alkyl (e.g., C₁-C₈, C₁-C₆, C₁-C₄, or C₁-C₂), R^{WW.1}-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g., 1, 2, 3, 1A, 2A, 3A, 1B, 2B, 3B, etc.). R^{WW.1}, R^{WW.2}, and R^{WW.3} are as defined above.

In the event that any L linker group recited in a claim or chemical formula description set forth herein (i.e., an L^{WW} substituent) is not explicitly defined, then that L group (L^{WW} group) is herein defined as independently a bond, —O—, —NH—, —C(O)—, —C(O)NH—, —NHC(O)—,
—NHC(O)NH—, —C(O)O—, —OC(O)—, —S—, —SO₂—, —SO₂NH—, R^{LWW.1}-substituted or unsubstituted alkylene (e.g., C₁-C₈, C₁-C₆, C₁-C₄, or C₁-C₂), R^{LWW.1}-substituted or unsubstituted heteroalkylene (e.g., 2 to 8 membered, 2 to 6 membered. 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{LWW.1}, as well as R^{LWW.2} and R^{LWW.3} are as defined above.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C— or ¹⁴C— enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego. For example, useful detectable agents include ¹⁸F, ³²p, ³³p, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As ⁸⁶y, ⁹⁰y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide specifically reactive with a target peptide. A detectable moiety is a monovalent detectable agent or a detectable agent capable of forming a bond with another composition.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²23Ra, and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb, and Lu.

Examples of detectable agents (e.g. detectable moieties) include imaging agents, including fluorescent and luminescent substances, molecules, or compositions, including, but not limited to, a variety of organic or inorganic small molecules commonly referred to as “dyes,” “labels,” or “indicators.” Examples include fluorescein, rhodamine, acridine dyes, Alexa dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent moiety or fluorescent dye moiety. In embodiments, the detectable moiety is a fluorescein isothiocyanate moiety, tetramethylrhodamine-5-(and 6)-isothiocyanate moiety, Cy2 moiety, Cy3 moiety, Cy5 moiety, Cy7 moiety, 4′,6-diamidino-2-phenylindole moiety, Hoechst 33258 moiety, Hoechst 33342 moiety, Hoechst 34580 moiety, propidium-iodide moiety, or acridine orange moiety. In embodiments, the detectable moiety is a Indo-1, Ca saturated moiety, Indo-1 Ca2+ moiety, Cascade Blue BSA pH 7.0 moiety, Cascade Blue moiety, LysoTracker Blue moiety, Alexa 405 moiety, LysoSensor Blue pH 5.0 moiety, LysoSensor Blue moiety, DyLight 405 moiety, DyLight 350 moiety, BFP (Blue Fluorescent Protein) moiety, Alexa 350 moiety, 7-Amino-4-methylcoumarin pH 7.0 moiety, Amino Coumarin moiety, AMCA conjugate moiety, Coumarin moiety, 7-Hydroxy-4-methylcoumarin moiety, 7-Hydroxy-4-methylcoumarin pH 9.0 moiety, 6,8-Difluoro-7-hydroxy-4-methylcoumarin pH 9.0 moiety, Hoechst 33342 moiety, Pacific Blue moiety, Hoechst 33258 moiety, Hoechst 33258-DNA moiety, Pacific Blue antibody conjugate pH 8.0 moiety, PO-PRO-1 moiety, PO-PRO-1-DNA moiety, POPO-1 moiety, POPO-1-DNA moiety, DAPI-DNA moiety, DAPI moiety, Marina Blue moiety, SYTOX Blue-DNA moiety, CFP (Cyan Fluorescent Protein) moiety, eCFP (Enhanced Cyan Fluorescent Protein) moiety, 1-Anilinonaphthalene-8-sulfonic acid (1,8-ANS) moiety, Indo-1, Ca free moiety, 1,8-ANS (1-Anilinonaphthalene-8-sulfonic acid) moiety, BO-PRO-1-DNA moiety, BOPRO-1 moiety, BOBO-1-DNA moiety, SYTO 45-DNA moiety, evoglow-Pp1 moiety, evoglow-Bs1 moiety, evoglow-Bs2 moiety, Auramine 0 moiety, DiO moiety, LysoSensor Green pH 5.0 moiety, Cy 2 moiety, LysoSensor Green moiety, Fura-2, high Ca moiety, Fura-2 Ca2+ sup> moiety, SYTO 13-DNA moiety, YO-PRO-1-DNA moiety, YOYO-1-DNA moiety, eGFP (Enhanced Green Fluorescent Protein) moiety, LysoTracker Green moiety, GFP (S65T) moiety, BODIPY FL, MeOH moiety, Sapphire moiety, BODIPY FL conjugate moiety, MitoTracker Green moiety, MitoTracker Green FM, MeOH moiety, Fluorescein 0.1 M NaOH moiety, Calcein pH 9.0 moiety, Fluorescein pH 9.0 moiety, Calcein moiety, Fura-2, no Ca moiety, Fluo-4 moiety, FDA moiety, DTAF moiety, Fluorescein moiety. CFDA moiety, FITC moiety, Alexa Fluor 488 hydrazide-water moiety, DyLight 488 moiety, 5-FAM pH 9.0 moiety, Alexa 488 moiety. Rhodamine 110 moiety. Rhodamine 110 pH 7.0 moiety, Acridine Orange moiety, BCECF pH 5.5 moiety, PicoGreendsDNA quantitation reagent moiety, SYBR Green I moiety, Rhodaminen Green pH 7.0 moiety, CyQUANT GR-DNA moiety, NeuroTrace 500/525, green fluorescent Nissl stain-RNA moiety, DansylCadaverine moiety, Fluoro-Emerald moiety, Nissl moiety, Fluorescein dextran pH 8.0 moiety, Rhodamine Green moiety, 5-(and-6)-Carboxy-2′, 7′-dichlorofluorescein pH 9.0 moiety, DansylCadaverine, MeOH moiety, eYFP (Enhanced Yellow Fluorescent Protein) moiety, Oregon Green 488 moiety, Fluo-3 moiety, BCECF pH 9.0 moiety, SBFI-Na+ moiety, Fluo-3 Ca2+ moiety, Rhodamine 123 MeOH moiety, F1AsH moiety, Calcium Green-1 Ca2+ moiety, Magnesium Green moiety, DM-NERF pH 4.0 moiety, Calcium Green moiety, Citrine moiety. LysoSensor Yellow pH 9.0 moiety, TO-PRO-1-DNA moiety, Magnesium Green Mg2+ moiety, Sodium Green Na+ moiety, TOTO-1-DNA moiety, Oregon Green 514 moiety, Oregon Green 514 antibody conjugate pH 8.0 moiety, NBD-X moiety, DM-NERF pH 7.0 moiety, NBD-X, MeOH moiety, CI-NERF pH 6.0 moiety, Alexa 430 moiety, CI-NERF pH 2.5 moiety, Lucifer Yellow, CH moiety, LysoSensor Yellow pH 3.0 moiety, 6-TET, SE pH 9.0 moiety, Eosin antibody conjugate pH 8.0 moiety, Eosin moiety, 6-Carboxyrhodamine 6G pH 7.0 moiety, 6-Carboxyrhodamine 6G, hydrochloride moiety, Bodipy R6G SE moiety, BODIPY R6G MeOH moiety, 6 JOE moiety, Cascade Yellow moiety, mBanana moiety, Alexa 532 moiety, Erythrosin-5-isothiocyanate pH 9.0 moiety, 6-HEX, SE pH 9.0 moiety, mOrange moiety, mHoneydew moiety, Cy 3 moiety, Rhodamine B moiety, DiI moiety, 5-TAMRA-MeOH moiety, Alexa 555 moiety, DyLight 549 moiety, BODIPY TMR-X, SE moiety, BODIPY TMR-X MeOH moiety, PO-PRO-3-DNA moiety, PO-PRO-3 moiety, Rhodamine moiety, POPO-3 moiety, Alexa 546 moiety, Calcium Orange Ca2+ moiety, TRITC moiety, Calcium Orange moiety, Rhodaminephalloidin pH 7.0 moiety, MitoTracker Orange moiety, MitoTracker Orange MeOH moiety, Phycoerythrin moiety, Magnesium Orange moiety, R-Phycoerythrin pH 7.5 moiety, 5-TAMRA pH 7.0 moiety, 5-TAMRA moiety, Rhod-2 moiety, FM 1-43 moiety, Rhod-2 Ca2+ moiety, FM 1-43 lipid moiety, LOLO-1-DNA moiety, dTomato moiety, DsRed moiety, Dapoxyl (2-aminoethyl) sulfonamide moiety, Tetramethylrhodamine dextran pH 7.0 moiety, Fluor-Ruby moiety, Resorufin moiety, Resorufin pH 9.0 moiety, mTangerine moiety, LysoTracker Red moiety, Lissaminerhodamine moiety, Cy 3.5 moiety, Rhodamine Red-X antibody conjugate pH 8.0 moiety, Sulforhodamine 101 EtOH moiety, JC-1 pH 8.2 moiety, JC-1 moiety, mStrawberry moiety, MitoTracker Red moiety, MitoTracker Red, MeOH moiety, X-Rhod-1 Ca2+ moiety, Alexa 568 moiety, 5-ROX pH 7.0 moiety, 5-ROX (5-Carboxy-X-rhodamine, triethylammonium salt) moiety, BO-PRO-3-DNA moiety, BOPRO-3 moiety, BOBO-3-DNA moiety, Ethidium Bromide moiety, ReAsH moiety, Calcium Crimson moiety, Calcium Crimson Ca2+ moiety, mRFP moiety, mCherry moiety, HcRed moiety, DyLight 594 moiety, Ethidium homodimer-1-DNA moiety, Ethidiumhomodimer moiety, Propidium Iodide moiety, SYPRO Ruby moiety, Propidium Iodide-DNA moiety, Alexa 594 moiety, BODIPY TR-X, SE moiety, BODIPY TR-X. MeOH moiety, BODIPY TR-X phallacidin pH 7.0 moiety, Alexa Fluor 610 R-phycoerythrin streptavidin pH 7.2 moiety, YO-PRO-3-DNA moiety, Di-8 ANEPPS moiety, Di-8-ANEPPS-lipid moiety, YOYO-3-DNA moiety, Nile Red-lipid moiety, Nile Red moiety, DyLight 633 moiety, mPlum moiety, TO-PRO-3-DNA moiety, DDAO pH 9.0 moiety, Fura Red high Ca moiety, Allophycocyanin pH 7.5 moiety, APC (allophycocyanin) moiety, Nile Blue, EtOH moiety, TOTO-3-DNA moiety, Cy 5 moiety, BODIPY 650/665-X, MeOH moiety, Alexa Fluor 647 R-phycoerythrin streptavidin pH 7.2 moiety, DyLight 649 moiety, Alexa 647 moiety, Fura Red Ca2+ moiety, Atto 647 moiety, Fura Red, low Ca moiety, Carboxynaphthofluorescein pH 10.0 moiety, Alexa 660 moiety, Cy 5,5 moiety, Alexa 680 moiety, DyLight 680 moiety, Alexa 700 moiety, FM 4-64, 2% CHAPS moiety, or FM 4-64 moiety. In embodiments, the dectable moiety is a moiety of 1,1-Diethyl-4,4-carbocyanine iodide, 1,2-Diphenylacetylene, 1,4-Diphenylbutadiene, 1,4-Diphenylbutadiyne, 1,6-Diphenylhexatriene, 1,6-Diphenylhexatriene, 1-anilinonaphthalene-8-sulfonic acid, 2,7-Dichlorofluorescein, 2,5-DIPHENYLOXAZOLE, 2-Di-1-ASP, 2-dodecylresorufin, 2-Methylbenzoxazole, 3,3-Diethylthiadicarbocyanine iodide, 4-Dimethylamino-4-Nitrostilbene, 5(6)-Carboxyfluorescein, 5(6)-Carboxynaphtofluorescein, 5(6)-Carboxytetramethylrhodamine B, 5-(and-6)-carboxy-2′,7′-dichlorofluorescein., 5-(and-6)-carboxy-2,7′-dichlorofluorescein, 5-(N-hexadecanoyl)aminoeosin, 5-(N-hexadecanoyl)aminoeosin, 5-chloromethylfluorescein, 5-FAM, 5-ROX, 5-TAMRA, 5-TAMRA, 6,8-difluoro-7-hydroxy-4-methylcoumarin, 6,8-difluoro-7-hydroxy-4-methylcoumarin, 6-carboxyrhodamine 6G, 6-HEX, 6-JOE, 6-JOE, 6-TET, 7-aminoactinomycin D, 7-Benzylamino-4-Nitrobenz-2-Oxa-1,3-Diazole, 7-Methoxycoumarin-4-Acetic Acid, 8-Benzyloxy-5,7-diphenylquinoline, 8-Benzyloxy-5,7-diphenylquinoline, 9,10-Bis(Phenylethynyl)Anthracene, 9,10-Diphenylanthracene, 9-METHYLCARBAZOLE, (CS)2Ir(μ—Cl)2Ir(CS)2, AAA, Acridine Orange, Acridine Orange, Acridine Yellow, Acridine Yellow, Adams Apple Red 680, Adirondack Green 520, Alexa Fluor 350, Alexa Fluor 405, Alexa Fluor 430, Alexa Fluor 430, Alexa Fluor 480, Alexa Fluor 488, Alexa Fluor 488, Alexa Fluor 488 hydrazide, Alexa Fluor 500, Alexa Fluor 514, Alexa Fluor 532, Alexa Fluor 546, Alexa Fluor 546, Alexa Fluor 555, Alexa Fluor 555, Alexa Fluor 568, Alexa Fluor 594, Alexa Fluor 594, Alexa Fluor 594, Alexa Fluor 610, Alexa Fluor 610-R-PE, Alexa Fluor 633, Alexa Fluor 635, Alexa Fluor 647, Alexa Fluor 647, Alexa Fluor 647-R-PE, Alexa Fluor 660, Alexa Fluor 680, Alexa Fluor 680-APC, Alexa Fluor 680-R-PE, Alexa Fluor 700, Alexa Fluor 750, Alexa Fluor 790, Allophycocyanin, AmCyan1, Aminomethylcoumarin, Amplex Gold (product), Amplex Red Reagent, Amplex UltraRed, Anthracene, APC, APC-Seta-750, AsRed2, ATTO 390, ATTO 425, ATTO 430LS, ATTO 465, ATTO 488, ATTO 490LS, ATTO 495, ATTO 514, ATTO 520, ATTO 532, ATTO 550, ATTO 565, ATTO 590, ATTO 594, ATTO 610, ATTO 620, ATTO 633, ATTO 635, ATTO 647, ATTO 647N, ATTO 655, ATTO 665, ATTO 680, ATTO 700, ATTO 725, ATTO 740, ATTO Oxal2, ATTO Rho3B, ATTO Rho6G, ATTO Rho11, ATTO Rho12, ATTO Rho13, ATTO Rho14, ATTO Rho101, ATTO Thio12, Auramine O, Azami Green, Azami Green monomeric, B-phycoerythrin, BCECF, BCECF, Bex1, Biphenyl, Birch Yellow 580, Blue-green algae, BO-PRO-1, BO-PRO-3, BOBO-1, BOBO-3, BODIPY 630 650-X, BODIPY 650/665-X, BODIPY FL, BODIPY FL, BODIPY R6G, BODIPY TMR-X, BODIPY TR-X, BODIPY TR-X Ph 7.0, BODIPY TR-X phallacidin, BODIPY-DiMe, BODIPY-Phenyl, BODIPY-TMSCC, C3-Indocyanine, C3-Indocyanine, C3-Oxacyanine, C3-Thiacyanine Dye (EtOH), C3-Thiacyanine Dye (PrOH), C5-Indocyanine, C5-Oxacyanine, C5-Thiacyanine, C7-Indocyanine, C7-Oxacyanine, C545T, C-Phycocyanin, Calcein, Calcein red-orange, Calcium Crimson, Calcium Green-1, Calcium Orange, Calcofluor white 2MR, Carboxy SNARF-1 pH 6.0, Carboxy SNARF-1 pH 9.0, Carboxynaphthofluorescein, Cascade Blue, Cascade Yellow, Catskill Green 540, CBQCA, CellMask Orange, CellTrace BODIPY TR methyl ester, CellTrace calcein violet, CellTrace™ Far Red, CellTracker Blue, CellTracker Red CMTPX, CellTracker Violet BMQC, CF405M, CF405S, CF488A, CF543, CF555, CFP, CFSE, CF™ 350, CF™ 485, Chlorophyll A, Chlorophyll B, Chromeo 488, Chromeo 494, Chromeo 505, Chromeo 546, Chromeo 642, Citrine, Citrine, ClOH butoxy aza-BODIPY, ClOH C12 aza-BODIPY, CM-H2DCFDA, Coumarin 1, Coumarin 6, Coumarin 6, Coumarin 30, Coumarin 314, Coumarin 334, Coumarin 343, Coumarine 545T, Cresyl Violet Perchlorate, CryptoLight CF1, CryptoLight CF2, CryptoLight CF3, CryptoLight CF4, CryptoLight CF5, CryptoLight CF6, Crystal Violet, Cumarin153, Cy2, Cy3, Cy3, Cy3.5, Cy3B, Cy3B, Cy3Cy5 ET, Cy5, Cy5, Cy5.5, Cy7, Cyanine3 NHS ester, Cyanine5 carboxylic acid, Cyanine5 NHS ester, Cyclotella meneghiniana Kutzing, CypHer5, CypHer5 pH 9.15, CyQUANT GR, CyTrak Orange, Dabcyl SE, DAF-FM, DAMC (Weiss), dansyl cadaverine, Dansyl Glycine (Dioxane), DAPI, DAPI, DAPI, DAPI, DAPI (DMSO), DAPI (H2O), Dapoxyl (2-aminoethyl)sulfonamide, DCI, DCM, DCM, DCM (acetonitrile), DCM (MeOH), DDAO, Deep Purple, di-8-ANEPPS, DiA, Dichlorotris(1,10-phenanthroline) ruthenium(II), DiClOH C12 aza-BODIPY, DiClOHbutoxy aza-BODIPY, DiD, DiI, DiIC18(3), DiO, DiR, Diversa Cyan-FP, Diversa Green-FP, DM-NERF pH 4.0, DOCI, Doxorubicin, DPP pH-Probe 590-7.5, DPP pH-Probe 590-9.0, DPP pH-Probe 590-11.0, DPP pH-Probe 590-11.0, Dragon Green, DRAQ5, DsRed, DsRed, DsRed, DsRed-Express, DsRed-Express2, DsRed-Express T1, dTomato, DY-350XL, DY-480, DY-480XL MegaStokes, DY-485, DY-485XL MegaStokes, DY-490, DY-490XL MegaStokes, DY-500, DY-500XL MegaStokes, DY-520, DY-520XL MegaStokes, DY-547, DY-549P1, DY-549P1, DY-554, DY-555, DY-557, DY-557, DY-590, DY-590, DY-615, DY-630, DY-631, DY-633, DY-635, DY-636, DY-647, DY-649P1, DY-649P1, DY-650, DY-651, DY-656, DY-673, DY-675, DY-676, DY-680, DY-681, DY-700, DY-701, DY-730, DY-731, DY-750, DY-751, DY-776, DY-782, Dye-28, Dye-33, Dye-45, Dye-304, Dye-1041, DyLight 488, DyLight 549. DyLight 594, DyLight 633, DyLight 649, DyLight 680, E2-Crimson, E2-Orange, E2-Red/Green, EBFP, ECF, ECFP, ECL Plus, eGFP, ELF 97, Emerald, Envy Green, Eosin, Eosin Y, epicocconone, EqFP611, Erythrosin-5-isothiocyanate, Ethidium bromide, ethidium homodimer-1, Ethyl Eosin, Ethyl Eosin, Ethyl Nile Blue A, Ethyl-p-Dimethylaminobenzoate, Ethyl-p-Dimethylaminobenzoate, Eu2O3 nanoparticles, Eu (Soini), Eu(tta)3DEADIT, EvaGreen, EVOblue-30, EYFP, FAD, FITC, FITC, F1AsH (Adams), Flash Red EX, F1AsH-CCPGCC, F1AsH-CCXXCC, Fluo-3, Fluo-4, Fluo-5F, Fluorescein, Fluorescein 0.1 NaOH, Fluorescein-Dibase, fluoro-emerald, Fluorol 5G, FluoSpheres blue, FluoSpheres crimson, FluoSpheres dark red, FluoSpheres orange, FluoSpheres red, FluoSpheres yellow-green, FM4-64 in CTC, FM4-64 in SDS, FM 1-43, FM 4-64, Fort Orange 600, Fura Red, Fura Red Ca free, fura-2, Fura-2 Ca free, Gadodiamide, Gd-Dtpa-Bma, Gadodiamide, Gd-Dtpa-Bma, GelGreen™, GelRed™, H9-40, HcRed1, Hemo Red 720, HiLyte Fluor 488, HiLyte Fluor 555, HiLyte Fluor 647, HiLyte Fluor 680, HiLyte Fluor 750, HiLyte Plus 555, HiLyte Plus 647, HiLyte Plus 750, HmGFP, Hoechst 33258, Hoechst 33342, Hoechst-33258, Hoechst-33258. Hops Yellow 560, HPTS, HPTS, HPTS, HPTS, HPTS, indo-1, Indo-1 Ca free, Ir(Cn)2(acac), Ir(Cs)2(acac), IR-775 chloride, IR-806, Ir-OEP-CO—Cl, IRDye® 650 Alkyne, IRDye® 650 Azide, IRDye® 650 Carboxylate, IRDye® 650 DBCO, IRDye® 650 Maleimide, IRDye® 650 NHS Ester, IRDye® 680LT Carboxylate, IRDye® 680LT Maleimide, IRDye® 680LT NHS Ester, IRDye® 680RD Alkyne, IRDye® 680RD Azide, IRDye® 680RD Carboxylate, IRDye® 680RD DBCO, IRDye® 680RD Maleimide, IRDye® 680RD NHS Ester, IRDye® 700 phosphoramidite, IRDye® 700DX, IRDye® 700DX, IRDye® 700DX Carboxylate, IRDye® 700DX NHS Ester, IRDye® 750 Carboxylate, IRDye® 750 Maleimide, IRDye® 750 NHS Ester, IRDye® 800 phosphoramidite, IRDye® 800CW, IRDye®800CW Alkyne, IRDye® 800CW Azide, IRDye® 800CW Carboxylate, IRDye®800CW DBCO, IRDye® 800CW Maleimide, IRDye® 800CW NHS Ester, IRDye®800RS, IRDye® 800RS Carboxylate, IRDye® 800RS NHS Ester, IRDye® QC-1 Carboxylate, IRDye® QC-1 NHS Ester, Isochrysis galbana-Parke, JC-1, JC-1, JOJO-1, Jonamac Red Evitag T2, Kaede Green, Kaede Red, kusabira orange, Lake Placid 490, LDS 751, Lissamine Rhodamine (Weiss), LOLO-1, lucifer yellow CH, Lucifer Yellow CH, lucifer yellow CH, Lucifer Yellow CH Dilitium salt, Lumio Green, Lumio Red, Lumogen F Orange, Lumogen Red F300, Lumogen Red F300, LysoSensor Blue DND-192, LysoSensor Green DND-153, LysoSensor Green DND-153, LysoSensor Yellow/Blue DND-160 pH 3, LysoSensor YellowBlue DND-160, LysoTracker Blue DND-22, LysoTracker Blue DND-22, LysoTracker Green DND-26, LysoTracker Red DND-99, LysoTracker Yellow HCK-123, Macoun Red Evitag T2, Macrolex Fluorescence Red G, Macrolex Fluorescence Yellow 10GN, Macrolex Fluorescence Yellow 10GN, Magnesium Green, Magnesium Octaethylporphyrin, Magnesium Orange, Magnesium Phthalocyanine, Magnesium Phthalocyanine, Magnesium Tetramesitylporphyrin, Magnesium Tetraphenylporphyrin, malachite green isothiocyanate, Maple Red-Orange 620, Marina Blue, mBanana, mBBr, mCherry, Merocyanine 540, Methyl green, Methyl green, Methyl green, Methylene Blue, Methylene Blue, mHoneyDew, MitoTracker Deep Red 633, MitoTracker Green FM, MitoTracker Orange CMTMRos, MitoTracker Red CMXRos, monobromobimane, Monochlorobimane, Monoraphidium, mOrange, mOrange2, mPlum, mRaspberry, mRFP, mRFP1, mRFP1.2 (Wang), mStrawberry (Shaner), mTangerine (Shaner), N,N-Bis(2,4,6-trimethylphenyl)-3,4:9,10-perylenebis(dicarboximide), NADH, Naphthalene, Naphthalene, Naphthofluorescein, Naphthofluorescein, NBD-X, NeuroTrace 500525, Nilblau perchlorate, nile blue, Nile Blue, Nile Blue (EtOH), nile red, Nile Red, Nile Red, Nile red, Nileblue A, NIR1, NIR2, NIR3, NIR4, NIR820, Octaethylporphyrin, OH butoxy aza-BODIPY, OHC12 aza-BODIPY, Orange Fluorescent Protein, Oregon Green 488, Oregon Green 488 DHPE, Oregon Green 514, Oxazin1, Oxazin 750, Oxazine 1, Oxazine 170, P4-3, P-Quaterphenyl, P-Terphenyl, PA-GFP (post-activation), PA-GFP (pre-activation), Pacific Orange, Palladium(II) meso-tetraphenyl-tetrabenzoporphyrin, PdOEPK, PdTFPP, PerCP-Cy5.5, Perylene, Perylene, Perylene bisimide pH-Probe 550-5.0, Perylene bisimide pH-Probe 550-5.5, Perylene bisimide pH-Probe 550-6.5, Perylene Green pH-Probe 720-5.5, Perylene Green Tag pH-Probe 720-6.0, Perylene Orange pH-Probe 550-2.0, Perylene Orange Tag 550, Perylene Red pH-Probe 600-5.5, Perylenediimid, Perylne Green pH-Probe 740-5.5, Phenol, Phenylalanine, pHrodo, succinimidyl ester, Phthalocyanine, PicoGreen dsDNA quantitation reagent, Pinacyanol-Iodide, Piroxicam, Platinum(II) tetraphenyltetrabenzoporphyrin, Plum Purple, PO-PRO-1, PO-PRO-3, POPO-1, POPO-3, POPOP, Porphin, PPO, Proflavin, PromoFluor-350, PromoFluor-405, PromoFluor-415, PromoFluor-488, PromoFluor-488 Premium, PromoFluor-488LSS, PromoFluor-500LSS, PromoFluor-505, PromoFluor-510LSS, PromoFluor-514LSS, PromoFluor-520LSS, PromoFluor-532, PromoFluor-546, PromoFluor-555, PromoFluor-590, PromoFluor-610, PromoFluor-633, PromoFluor-647, PromoFluor-670, PromoFluor-680, PromoFluor-700, PromoFluor-750, PromoFluor-770, PromoFluor-780, PromoFluor-840, propidium iodide, Protoporphyrin IX, PTIR475/UF, PTIR545/UF, PtOEP, PtOEPK, PtTFPP, Pyrene, QD525, QD565, QD585, QD605, QD655, QD705, QD800, QD903, QD PbS 950, QDot 525, QDot 545, QDot 565, Qdot 585, Qdot 605, Qdot 625, Qdot 655, Qdot 705, Qdot 800, QpyMe2, QSY 7, QSY 7, QSY 9, QSY 21, QSY 35, quinine, Quinine Sulfate, Quinine sulfate, R-phycoerythrin, R-phycoerythrin, ReAsH-CCPGCC, ReAsH-CCXXCC, Red Beads (Weiss), Redmond Red, Resorufin, resorufin, rhod-2, Rhodamin 700 perchlorate, rhodamine, Rhodamine 6G, Rhodamine 6G, Rhodamine 101, rhodamine 110, Rhodamine 123, rhodamine 123. Rhodamine B, Rhodamine B, Rhodamine Green, Rhodamine pH-Probe 585-7.0, Rhodamine pH-Probe 585-7.5, Rhodamine phalloidin, Rhodamine Red-X, Rhodamine Red-X, Rhodamine Tag pH-Probe 585-7.0, Rhodol Green, Riboflavin, Rose Bengal, Sapphire, SBFI, SBFI Zero Na, Scenedesmus sp., SensiLight PBXL-1, SensiLight PBXL-3, Seta 633-NHS, Seta-633-NHS, SeTau-380-NHS, SeTau-647-NHS, Snake-Eye Red 900, SNIR1, SNIR2, SNIR3, SNIR4, Sodium Green, Solophenyl flavine 7GFE 500, Spectrum Aqua, Spectrum Blue, Spectrum FRed, Spectrum Gold, Spectrum Green, Spectrum Orange, Spectrum Red, Squarylium dye III, Stains All, Stilben derivate, Stilbene, Styryl8 perchlorate, Sulfo-Cyanine3 carboxylic acid, Sulfo-Cyanine3 carboxylic acid, Sulfo-Cyanine3 NHS ester, Sulfo-Cyanine5 carboxylic acid, Sulforhodamine 101, sulforhodamine 101, Sulforhodamine B, Sulforhodamine G, Suncoast Yellow, SuperGlo BFP, SuperGlo GFP, Surf Green EX, SYBR Gold nucleic acid gel stain, SYBR Green I, SYPRO Ruby, SYTO 9, SYTO 11, SYTO 13, SYTO 16, SYTO 17, SYTO 45, SYTO 59, SYTO 60, SYTO 61, SYTO 62, SYTO 82, SYTO RNASelect, SYTO RNASelect, SYTOX Blue, SYTOX Green, SYTOX Orange, SYTOX Red, T-Sapphire, Tb (Soini), tCO, tdTomato, Terrylen, Terrylendiimid, testdye, Tetra-t-Butylazaporphine, Tetra-t-Butylnaphthalocyanine, Tetracen, Tetrakis(o-Aminophenyl)Porphyrin, Tetramesitylporphyrin, Tetramethylrhodamine, tetramethylrhodamine, Tetraphenylporphyrin, Tetraphenylporphyrin, Texas Red, Texas Red DHPE, Texas Red-X, ThiolTracker Violet, Thionin acetate, TMRE, TO-PRO-1, TO-PRO-3, Toluene, Topaz (Tsien1998), TOTO-1, TOTO-3, Tris(2,2-Bipyridyl)Ruthenium(II) chloride, Tris(4,4-diphenyl-2,2-bipyridine) ruthenium(II) chloride, Tris(4,7-diphenyl-1,10-phenanthroline) ruthenium(II) TMS, TRITC (Weiss), TRITC Dextran (Weiss), Tryptophan, Tyrosine, Vex1, Vybrant DyeCycle Green stain, Vybrant DyeCycle Orange stain, Vybrant DyeCycle Violet stain, WEGFP (post-activation), WellRED D2, WellRED D3, WellRED D4, WtGFP, WtGFP (Tsien1998), X-rhod-1, Yakima Yellow, YFP, YO-PRO-1, YO-PRO-3, YOYO-1, YoYo-1, YoYo-1 dsDNA, YoYo-1 ssDNA, YOYO-3, Zinc Octaethylporphyrin, Zinc Phthalocyanine, Zinc Tetramesitylporphyrin, Zinc Tetraphenylporphyrin, ZsGreen1, or ZsYellow1. In embodiments, R³ is a monovalent moiety of a compound described within this paragraph.

A “labeled protein or polypeptide” is one that is bound, either covalently, through a linker or a chemical bond, or noncovalently, through ionic, van der Waals, electrostatic, or hydrogen bonds to a label such that the presence of the labeled protein or polypeptide may be detected by detecting the presence of the label bound to the labeled protein or polypeptide. Alternatively, methods using high affinity interactions may achieve the same results where one of a pair of binding partners binds to the other, e.g., biotin, streptavidin.

The term “linker” as described herein is a divalent chemical group that joins one chemical moiety to another. In embodiments, the linker is a covalent linker. In embodiments, the linker is a non-covalent linker. Specific examples of linkers are described throughout the specification, including in the examples and figures. Any appropriate linker may be used, including a polyethylene (PEG) linker or equivalent, or a polymer linker known in the art.

As used herein, the term “bioconjugate” or “bioconjugate linker” refers to the resulting association between atoms or molecules of bioconjugate reactive groups. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g. a sulfhydryl). In embodiments, the first bioconjugate reactive group (e.g., haloacetyl moiety) is covalently attached to the second bioconjugate reactive group (e.g. a sulfhydryl). In embodiments, the first bioconjugate reactive group (e.g., pyridyl moiety) is covalently attached to the second bioconjugate reactive group (e.g. a sulfhydryl). In embodiments, the first bioconjugate reactive group (e.g., —N-hydroxysuccinimide moiety) is covalently attached to the second bioconjugate reactive group (e.g. an amine). In embodiments, the first bioconjugate reactive group (e.g., maleimide moiety) is covalently attached to the second bioconjugate reactive group (e.g. a sulfhydryl). In embodiments, the first bioconjugate reactive group (e.g., -sulfo-N-hydroxysuccinimide moiety) is covalently attached to the second bioconjugate reactive group (e.g. an amine).

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or
CH₃). Likewise, for a linker variable (e.g., L¹ and L² as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amio acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine.; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that may be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The terms “TGT protein”, “TGT enyzme”, “TGT”, and “tRNA-guanine transglycosylase enzyme” are used interchangeably, and include any of the recombinant or naturally-occurring forms of tRNA-guanine transglycosylase, also known as Queuine tRNA-ribosyltransferase, Guanine insertion enzyme, Queuine tRNA-ribosyltransferase or variants or homologs thereof that maintain TGT activity (e.g. within at least 50%, 80%, 90%, 95%, 96%, 97%, 98%, 99% or 100% activity compared to TGT). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring TGT protein. In embodiments, the TGT protein is substantially identical to the protein identified by the UniProt reference number P0A847 or a variant or homolog having substantial identity thereto. In embodiments, the TGT protein is substantially identical to the protein identified by the UniProt reference number P28720 or a variant or homolog having substantial identity thereto.

The “catalytic domain,” “catalytic site,” “binding site” or “active site” of bacterial transglycosylase is a region within the transglycosylase where substrate molecules bind to and undergo a chemical transformation (e.g. substitution of a guanine within a DNA molecule with a PreQ1 analog or derivative). The active site includes residues that form temporary bonds with the “substrate.”

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The term “plasmid” or “expression vector” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids.

A “cell” as used herein, refers to a cell carrying out metabolic or other functions sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein (e.g. TGT) in the absence of a compound as described herein (including embodiments and examples).

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound (e.g. DNA compound) as described herein and a protein or enzyme (e.g. TGT). In some embodiments contacting includes allowing a compound described herein to interact with a protein or enzyme.

### DNA Compounds

Provided herein, inter alia, are DNA compounds including a PreQ1 analog or PreQ1 derivative thereof as provided herein including embodiments thereof. As described throughout the specification, PreQ1 analogs or derivatives are useful for modifying DNA molecules with functional moieties, including biomolecules, bioorthogonal handles, detectable moieties, and drug moieties. In instances, a PreQ1 analog (e.g. an alkyl amine derivative, etc.) includes a functional moiety (e.g. biomolecules, detectable moieties, drug moieties, etc.) attached to the exocyclic primary amine of PreQ1 via a linker, for example an alkyl chain linker or a polyethylene glycol (PEG) linker. In instances, a PreQ1 derivative includes a functional moiety (e.g. biomolecules, detectable moieties, drug moieties, etc.) attached to the C7 of PreQ1 via a linker, for example an alkyl chain linker or a polyethylene glycol (PEG) linker.A PreQ1 analog or derivative is capable of replacing a guanine nuclebase within a loop region of a hairpin within a DNA molecule, thereby generating the DNA compound as provided herein including embodiments thereof. This reaction can be carried out by a transglycosylase, for example, bacterial (E.coli) tRNA Guanine Transglycosylase (TGT) whose natural substrate is the small molecule PreQ1. Applicant has demonstrated that PreQ1 analog and derivatives can be successfully incorporated by the TGT enzyme into the loop portion of a DNA hairpin with comparable efficiency to that of the natural substrate.

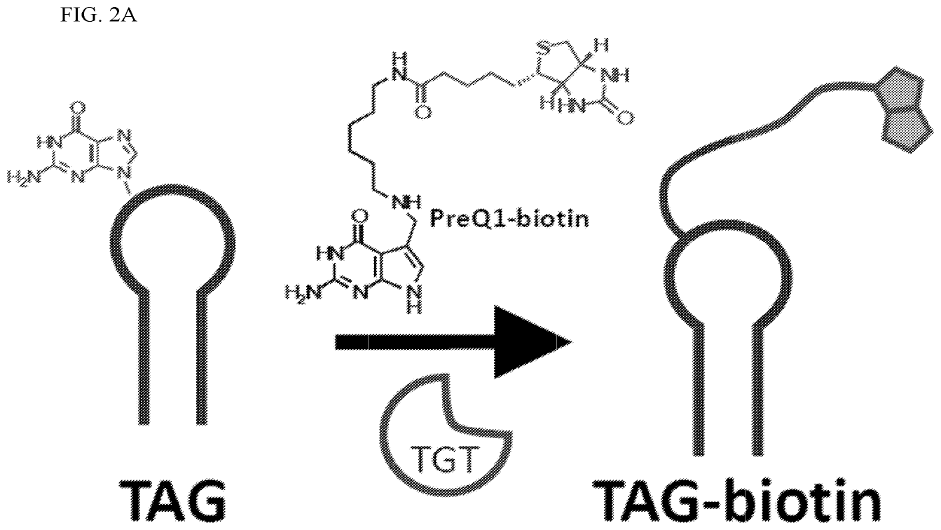

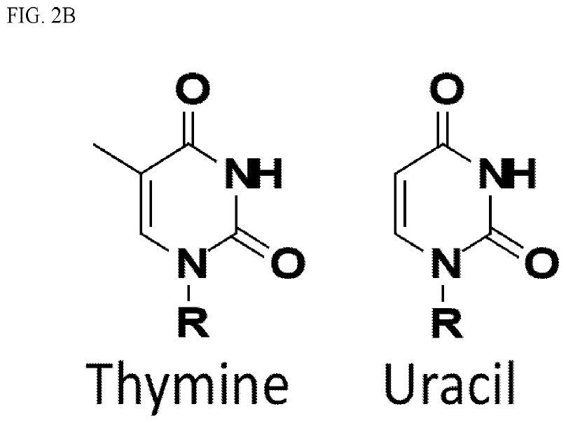

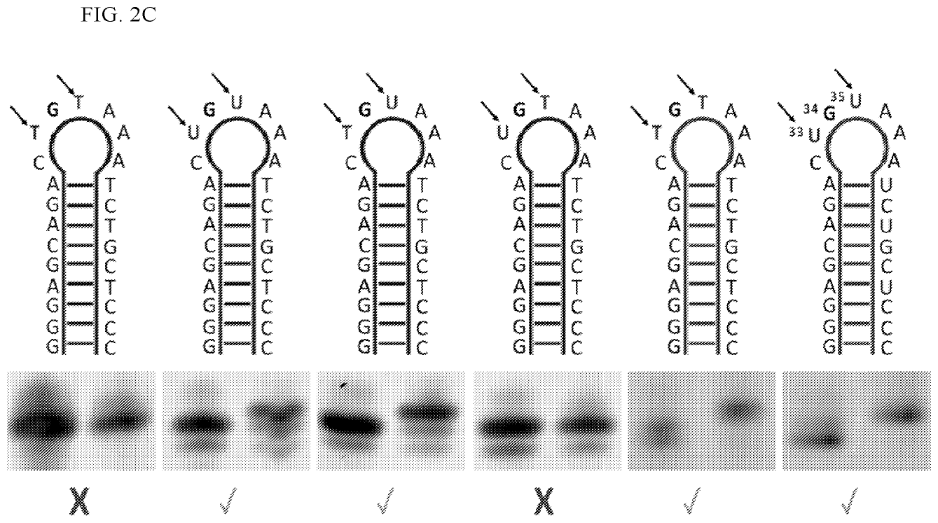

The terms “DNA hairpin”, “DNA stem loop”, and “hairpin” are interchangeable, are used in accordance to their ordinary meaning in the art, and refer to a region of a DNA oligonucleotide (e.g. DNA molecule) that that includes two nucleotide sequences that base pair to form a double-stranded structure (e.g. the stem) with a non-base paired structure (e.g. the loop, the loop portion) at one end of the double-stranded structure. DNA hairpins are typically formed in a single-stranded DNA when two regions of the same DNA strand at least partially complementary when read in opposite directions base-pair to form a double helix (the stem portion) that ends in an unpaired loop (the loop portion). DNA hairpins may form when duplex DNA undergoes supercoiling and a portion of the duplex separates. A strand of the separated portion may form a hairpin when complementary regions of the strand base pair. Example DNA hairpins are shown in FIG. 2C. The stem, also referred to as the “stem portion”, of the DNA hairpin is typically from about 4 to about 40 base pairs in length. The loop, also referred to as the “loop portion”, forms a non-based paired structure at one end of the DNA hairpin, and must be at least 3 nucleotides in length, and is typically from about 3 nucleotides to about 10 nucleotides in length. However, the loop may be longer, for example up to 50 nucleotides in length. Thus, the “loop” or “loop portion” caps one end of the DNA hairpin. As used herein, a “DNA molecule” may refer to a nucleic acid with at least one DNA hairpin structure.

In embodiments, the loop (e.g. loop portion) of the DNA hairpin is from about 3 nucleotides to about 30 nucleotides in length. In embodiments, the loop (e.g. loop portion) of the DNA hairpin is from about 6 nucleotides to about 30 nucleotides in length. In embodiments, the loop (e.g. loop portion) of the DNA hairpin is from about 9 nucleotides to about 30 nucleotides in length. In embodiments, the loop (e.g. loop portion) of the DNA hairpin is from about 12 nucleotides to about 30 nucleotides in length. In embodiments, the loop (e.g. loop portion) of the DNA hairpin is from about 15 nucleotides to about 30 nucleotides in length. In embodiments, the loop (e.g. loop portion) of the DNA hairpin is from about 18 nucleotides to about 30 nucleotides in length. In embodiments, the loop (e.g. loop portion) of the DNA hairpin is from about 21 nucleotides to about 30 nucleotides in length. In embodiments, the loop (e.g. loop portion) of the DNA hairpin is from about 24 nucleotides to about 30 nucleotides in length. In embodiments, the loop (e.g. loop portion) of the DNA hairpin is from about 27 nucleotides to about 30 nucleotides in length.

In embodiments, the loop (e.g. loop portion) of the DNA hairpin is from about 3 nucleotides to about 27 nucleotides in length. In embodiments, the loop (e.g. loop portion) of the DNA hairpin is from about 3 nucleotides to about 24 nucleotides in length. In embodiments, the loop (e.g. loop portion) of the DNA hairpin is from about 3 nucleotides to about 21 nucleotides in length. In embodiments, the loop (e.g. loop portion) of the DNA hairpin is from about 3 nucleotides to about 18 nucleotides in length. In embodiments, the loop (e.g. loop portion) of the DNA hairpin is from about 3 nucleotides to about 15 nucleotides in length. In embodiments, the loop (e.g. loop portion) of the DNA hairpin is from about 3 nucleotides to about 12 nucleotides in length. In embodiments, the loop (e.g. loop portion) of the DNA hairpin is from about 3 nucleotides to about 9 nucleotides in length. In embodiments, the loop (e.g. loop portion) of the DNA hairpin is from about 3 nucleotides to about 6 nucleotides in length. In embodiments, the loop (e.g. loop portion) of the DNA hairpin is about 3, 6, 9, 12, 15, 18, 21, 24, 27, or 30 nucleotides in length.

In embodiments, the loop (e.g. loop portion) of the DNA hairpin is from about 3 nucleotides to about 10 nucleotides in length. In embodiments, the loop (e.g. loop portion) of the DNA hairpin is from about 4 nucleotides to about 10 nucleotides in length. In embodiments, the loop (e.g. loop portion) of the DNA stem loop is from about 5 nucleotides to about 10 nucleotides in length. In embodiments, the loop (e.g. loop portion) of the DNA stem loop is from about 6 nucleotides to about 10 nucleotides in length. In embodiments, the loop (e.g. loop portion) of the DNA stem loop is from about 7 nucleotides to about 10 nucleotides in length. In embodiments, the loop (e.g. loop portion) of the DNA stem loop is from about 8 nucleotides to about 10 nucleotides in length. In embodiments, the loop (e.g. loop portion) of the DNA stem loop is from about 9 nucleotides to about 10 nucleotides in length.

In embodiments, the loop (e.g. loop portion) of the DNA hairpin is from about 3 nucleotides to about 9 nucleotides in length. In embodiments, the loop (e.g. loop portion) of the DNA hairpin is from about 3 nucleotides to about 8 nucleotides in length. In embodiments, the loop (e.g. loop portion) of the DNA hairpin is from about 3 nucleotides to about 7 nucleotides in length. In embodiments, the loop (e.g. loop portion) of the DNA hairpin is from about 3 nucleotides to about 6 nucleotides in length. In embodiments, the loop (e.g. loop portion) of the DNA hairpin is from about 3 nucleotides to about 5 nucleotides in length. In embodiments, the loop (e.g. loop portion) of the DNA hairpin is from about 3 nucleotides to about 4 nucleotides in length. In embodiments, the loop (e.g. loop portion) of the DNA hairpin is 3 nucleotides, 4 nucleotides, 5 nucleotides, 6 nucleotides, 7 nucleotides, 8 nucleotides, 9 nucleotides, or 10 nucleotides in length. In embodiments, the loop (e.g. loop portion) of the DNA hairpin is 4 nucleotides in length. In embodiments, the loop (e.g. loop portion) of the DNA hairpin is 5 nucleotides in length. In embodiments, the loop (e.g. loop portion) of the DNA hairpin is 6 nucleotides in length. In embodiments, the loop (e.g. loop portion) of the DNA hairpin is 7 nucleotides in length.

In embodiments, the stem (e.g. stem portion) of the DNA hairpin is from about 4 to about 40 base pairs in length. In embodiments, the stem (e.g. stem portion) of the DNA hairpin is from about 8 to about 40 base pairs in length. In embodiments, the stem (e.g. stem portion) of the DNA hairpin is from about 12 to about 40 base pairs in length. In embodiments, the stem (e.g. stem portion) of the DNA hairpin is from about 16 to about 40 base pairs in length. In embodiments, the stem (e.g. stem portion) of the DNA hairpin is from about 20 to about 40 base pairs in length. In embodiments, the stem (e.g. stem portion) of the DNA hairpin is from about 24 to about 40 base pairs in length. In embodiments, the stem (e.g. stem portion) of the DNA hairpin is from about 28 to about 40 base pairs in length. In embodiments, the stem (e.g. stem portion) of the DNA hairpin is from about 32 to about 40 base pairs in length. In embodiments, the stem (e.g. stem portion) of the DNA hairpin is from about 36 to about 40 base pairs in length.

In embodiments, the stem (e.g. stem portion) of the DNA hairpin is from about 4 to about 36 base pairs in length. In embodiments, the stem (e.g. stem portion) of the DNA hairpin is from about 4 to about 32 base pairs in length. In embodiments, the stem (e.g. stem portion) of the DNA hairpin is from about 4 to about 28 base pairs in length. In embodiments, the stem (e.g. stem portion) of the DNA hairpin is from about 4 to about 24 base pairs in length. In embodiments, the stem (e.g. stem portion) of the DNA hairpin is from about 4 to about 20 base pairs in length. In embodiments, the stem (e.g. stem portion) of the DNA hairpin is from about 4 to about 16 base pairs in length. In embodiments, the stem (e.g. stem portion) of the DNA hairpin is from about 4 to about 12 base pairs in length. In embodiments, the stem (e.g. stem portion) of the DNA hairpin is from about 4 to about 8 base pairs in length. In embodiments, the stem (e.g. stem portion) of the DNA hairpin is 4 base pairs, 8 base pairs, 12 base pairs, 16 base pairs, 20 base pairs, 24 base pairs, 28 base pairs, 32 base pairs, 36 base pairs, or 40 base pairs in length. In embodiments, the stem (e.g. stem portion) of the DNA hairpin is 5 base pairs in length. In embodiments, the stem (e.g. stem portion) of the DNA hairpin is 6 base pairs in length. In embodiments, the stem (e.g. stem portion) of the DNA hairpin is 7 base pairs in length. In embodiments, the stem (e.g. stem portion) of the DNA hairpin is 8 base pairs in length. In embodiments, the stem (e.g. stem portion) of the DNA hairpin is 9 base pairs in length. In embodiments, the stem (e.g. stem portion) of the DNA hairpin is 10 base pairs in length. In embodiments, the stem (e.g. stem portion) of the DNA hairpin is 11 base pairs in length.

The term “PreQ1 analog” as used herein, refers to a PreQ1 that is chemically modified at the nitrogen in the —CH₂NH₂— moiety of the PreQ1 nucleobase to form a secondary amine (e.g. —CH₂NHR—), where R is not hydrogen. PreQ1 analogs have the general structure:

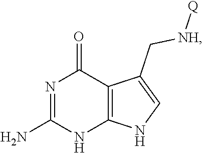

where Q is not hydrogen. In embodiments, Q is -L¹-L²-R³.

The term “PreQ1 derivative” as used herein, refers to a PreQ1 that is chemically modified at the C7 carbon of the PreQ1 nucleobase. PreQ1 derivatives have the general structure:

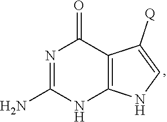

where Q is not hydrogen. In embodiments, Q is -L¹-L²-R³.

The structure of PreQ1 or PreQ1 nucleobase is as follows:

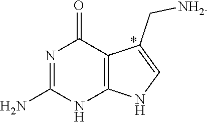 [SMILES: NCc1c[nH]c2[nH]c(N)nc(=O)c12]

The C7 carbon of PreQ1 is labeled with a *.

In an aspect is provided a DNA compound having the formula:

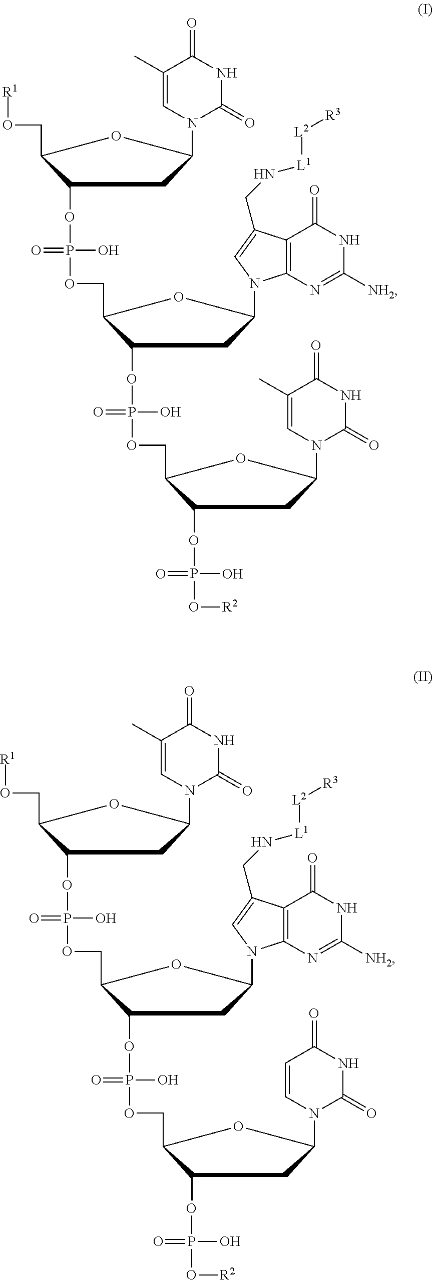

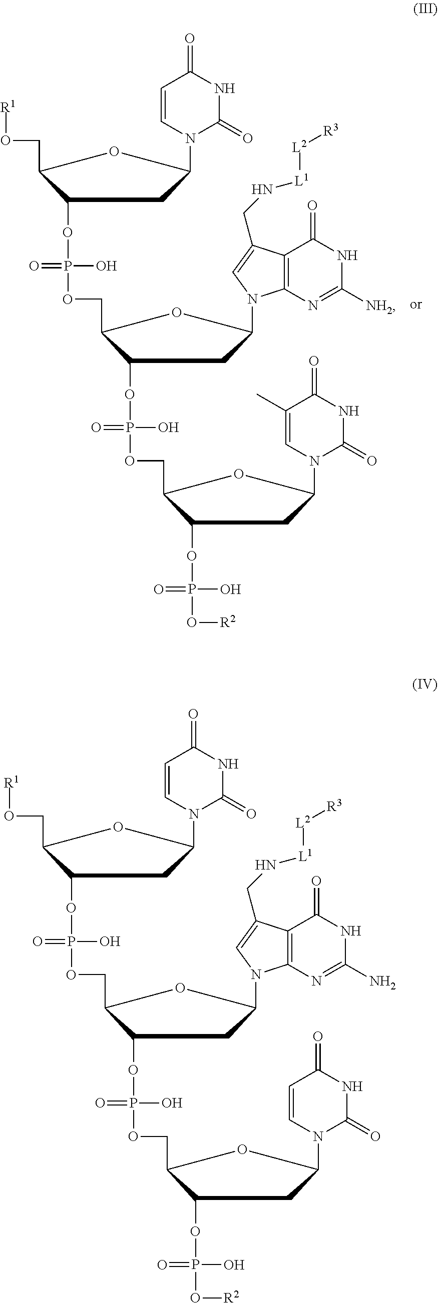

In another aspect is provided a DNA compound having the formula:

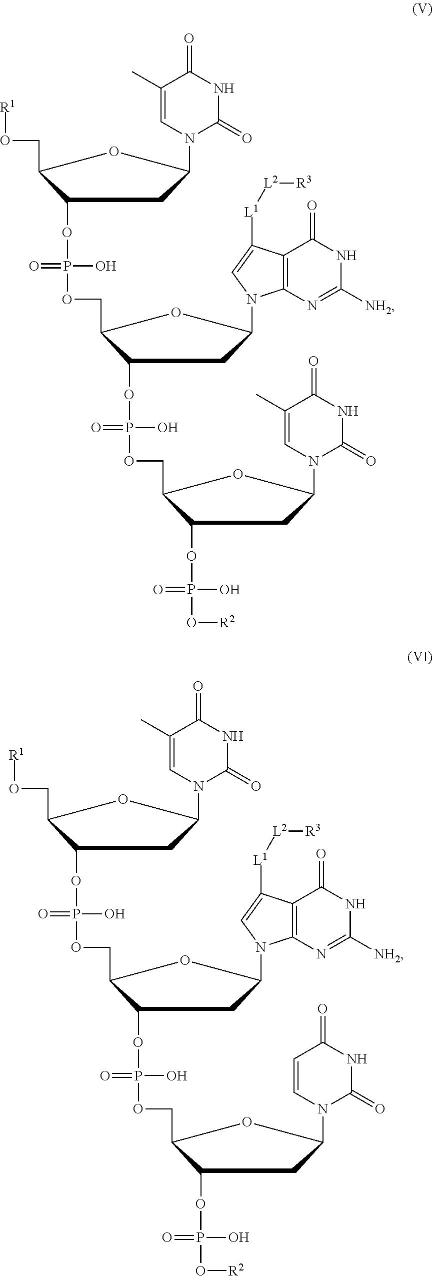

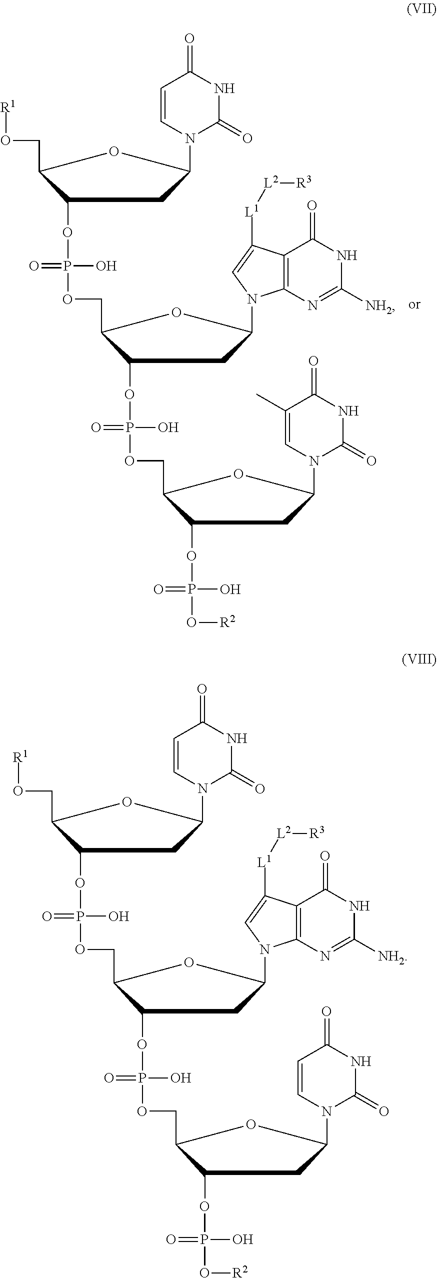

R¹ is hydrogen, a deoxynucleotide, or a first DNA sequence comprising the 5′ of a DNA hairpin. R² is hydrogen, a deoxynucleotide, or a second DNA sequence comprising the 3′ of the DNA hairpin.

L¹ and L² are independently a bond, —O—, —S—, —NH—, —C(O)NH₂, —NHC(O)—, —C(O)O—, —OC(O)—, —S(O)₂NH₂, —NHS(O)₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene, a peptide linker, or a cleavable linker.

R³ is a detectable moiety, a biomolecular moiety, a therapeutic moiety, hydrogen, halogen, —CX^{3.1}₃, —CHX^{3.1}₂, —CH₂X^{3.1}, —CN, —SOₙ₁R^{3A}, —SOᵥ₁NR^{3B}R^{3C}, —NHNR^{3B}R^{3C}, —ONR^{3B}R^{3C}, —NHC(O)NHNR^{3B}R^{3C}, —NHC(O)NR^{3B}R^{3C}, —N(O)ₘ₁, —NR^{3B}R^{3C}, —C(O)R^{3D}, —C(O)OR^{3D}, —C(O)NR^{3B}R^{3C}, —OR^{3A}, —NR^{3B}SO₂R^{3A}, —NR^{3B}C(O)R^{3D}, —NR^{3B}C(O)OR^{3D}, —NR^{3B}OR^{3D}, —OCX^{3.1}₂, —OCHX^{3.1}₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{3A}, R^{3B}, R^{3C} and R^{3D} are independently hydrogen, halogen, —CF₃, —CCl₃, —CBr₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3B} and R^{3C} substituents bonded to the same nitrogen atom may optionally be joined to form a substituted or unsubstituted heterocycloalkyl or substituted or unsubstituted heteroaryl.

X^{3.1} is independently-Cl, —Br, —I or —F.

In embodiments, the DNA compound has the formula:

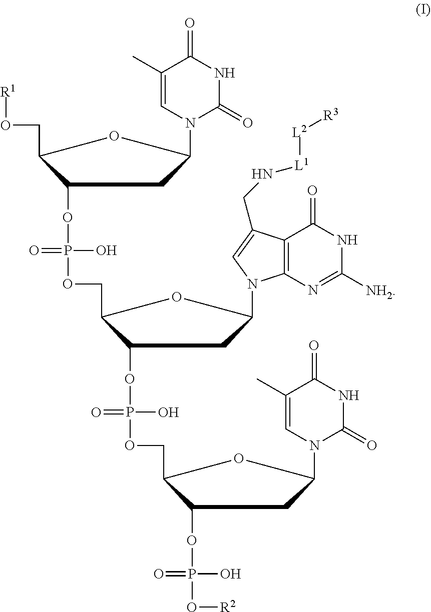

In embodiments, the DNA compound has the formula:

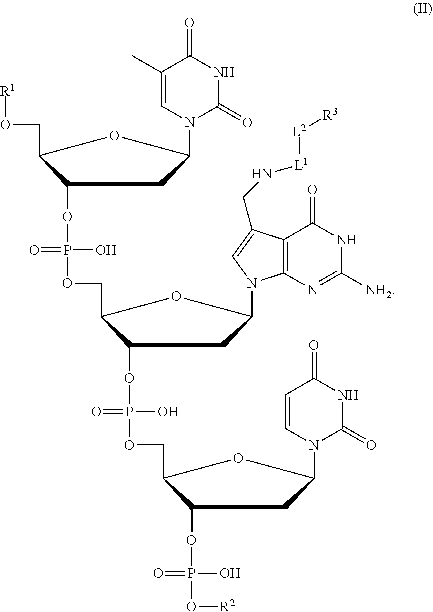

In embodiments, the DNA compound has the formula:

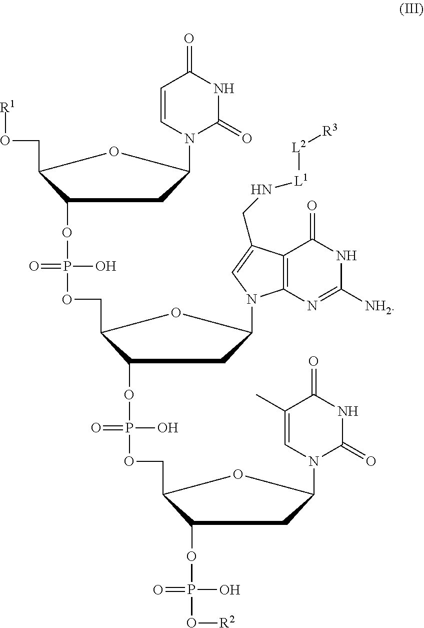

In embodiments, the DNA compound has the formula:

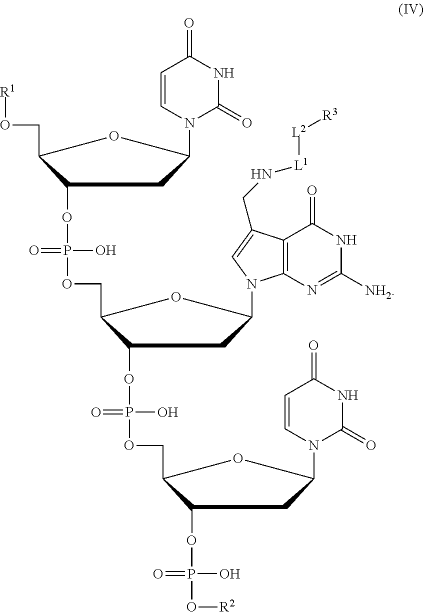

In embodiments, the DNA compound has the formula:

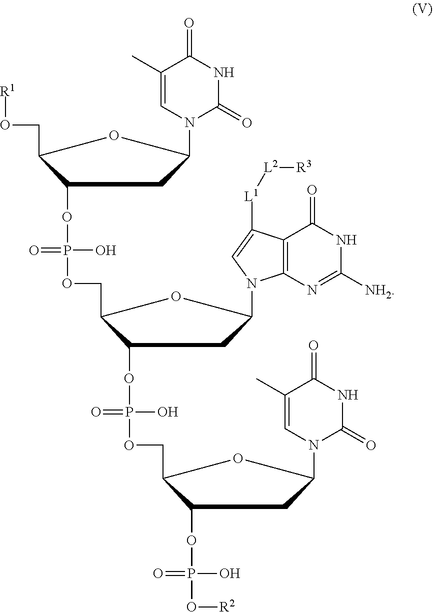

In embodiments, the DNA compound has the formula:

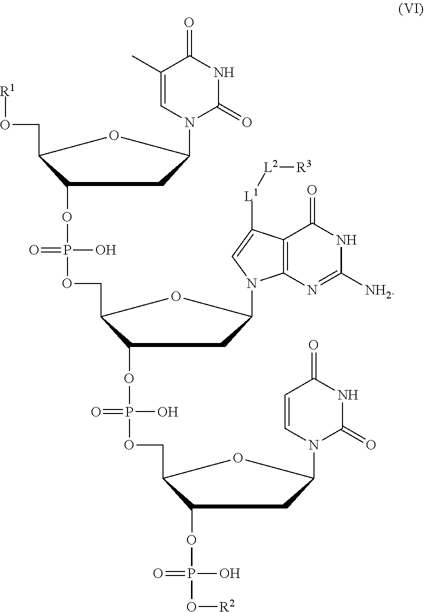

In embodiments, the DNA compound has the formula:

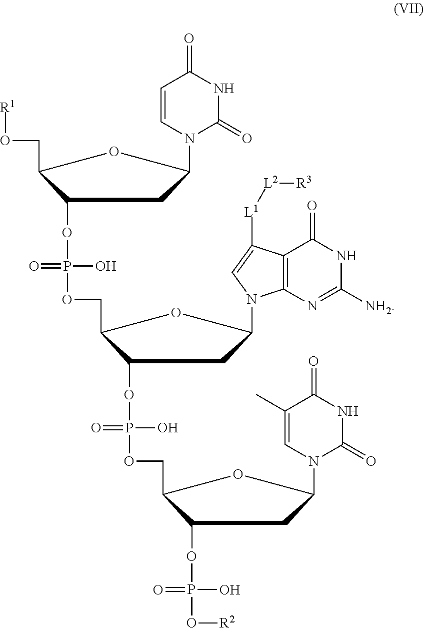

In embodiments, the DNA compound has the formula:

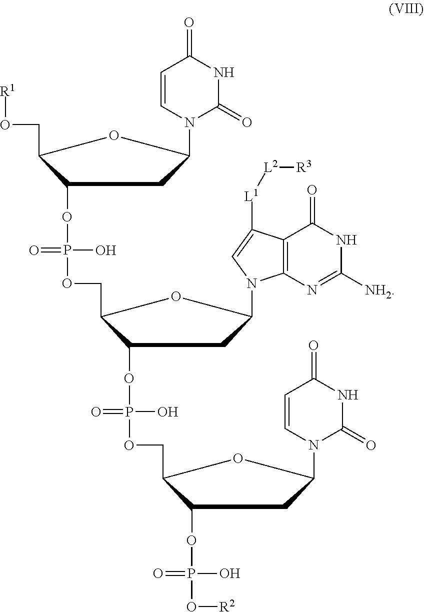

For the DNA compounds of formulas I-VIII, in embodiments, R¹ and R² are independently hydrogen. In embodiments, R¹ is hydrogen. In embodiments, R² is hydrogen.

For the DNA compounds of formulas I-VIII, in embodiments, R¹ and R² are independently a deoxynucleotide. In embodiments, R¹ is a deoxynucleotide. In embodiments, R² is a a deoxynucleotide. In embodiments, R¹ and R² are independently a deoxyadenosine, deoxyguanosine, thymidine, deoxyuridine, or deoxycytidine. In embodiments, R¹ is a deoxyadenosine. In embodiments, R¹ is a deoxyguanosine. In embodiments, R¹ is a thymidine. In embodiments, R¹ is a deoxycytidine. In embodiments, R² is a deoxyadenosine. In embodiments, R² is a deoxyguanosine. In embodiments, R² is a thymidine. In embodiments, R² is a deoxycytidine.

For the DNA compounds of formulas I-VIII, in embodiments, each of the phosphodiester intemucleotide linkages are independently substituted with a phosphodiester derivative. In embodiments, the phosphodiester derivative is a phosphoramidate, a phosphorodiamidate, a phosphorothioate, a phosphorodithioate, a phosphonocarboxylic acid, a phosphonocarboxylate, a phosphonoacetic acid, a phosphonoformic acid, a methyl phosphonate, a boron phosphonate, or a O— methylphosphoroamidite linkage.

In embodiments, the DNA compound (e.g. the DNA compounds of formulas I-VIII) provided herein including embodiments thereof includes a hairpin structure. In embodiments, the DNA compound provided herein is a hairpin structure. For example, the DNA compound may be a modified DNA molecule as provided herein including embodiments thereof, wherein the PreQ1 analog or derivative thereof is within the loop portion of the hairpin. Thus, in embodiments, R¹ includes the 5′ of a DNA hairpin as provided herein including embodiments thereof, and R² includes the 3′ of a DNA hairpin as provided herein including embodiments thereof. In embodiments, R¹ includes a 5′ loop portion of a DNA hairpin. In embodiments, R² includes the 3′ loop portion of the DNA hairpin. In embodiments, R¹ is the 5′ loop portion of a DNA hairpin. In embodiments, R² is the ₃′ loop portion of the DNA hairpin. In embodiments, R¹ and R² are independently a first DNA sequence including the 5′ of a DNA hairpin and a second DNA sequence including the 3′ of the DNA hairpin. In embodiments, R¹ includes a first DNA sequence including the 5′ of a DNA hairpin. In embodiments, R² includes a second DNA sequence including the 3′ of the DNA hairpin.

In embodiments, R¹ is hydrogen. In embodiments, R¹ is a deoxynucleotide. In embodiments, R¹ is a deoxyadenosine. In embodiments, R¹ is a deoxyguanosine. In embodiments, R¹ is a thymidine. In embodiments, R¹ is a deoxycytidine.

In embodiments, R² is hydrogen. In embodiments, R² is a deoxynucleotide. In embodiments, R² is a deoxyadenosine. In embodiments, R² is a deoxyguanosine. In embodiments, R² is a thymidine. In embodiments, R² is a deoxycytidine.

In embodiments, for the PreQ1 analog, PreQ1 derivative and DNA compounds of formulas I-VIII, a functional moiety (R³) may be attached to the PreQ1 analog, PreQ1 derivative or DNA compound. In embodiments, for the PreQ1 analog provided herein a functional moiety may be attached to the exocyclic primary amine of the PreQ1 analog within the DNA compound (e.g. DNA compounds of formulas I-IV) via covalent linkers, L¹ and L². In embodiments, a functional moiety (R³) may be attached to C7 of the PreQ1 derivative within the DNA compound (e.g. DNA compounds of formulas V-VIII) via covalent linkers, L¹ and L². L¹ and L² are independently a bond, —O—, —S—, —NH—, —C(O)NH₂, —NHC(O)—, —C(O)O—, —OC(O)—, —S(O)₂NH₂, —NHS(O)₂—, substituted or unsubstituted alkylene (e.g., C₁-C₈, C₁-C₆, or C₁-C₄), substituted or unsubstituted heteroalkylene (e.g., 2 to 8 membered, 2 to 6 membered, or 2 to 4 membered), substituted or unsubstituted cycloalkylene (e.g., C₃-C₈, C₃-C₆, or C₅-C₆), substituted or unsubstituted heterocycloalkylene (e.g., 3 to 8 membered, 3 to 6 membered, or 5 to 6 membered), substituted or unsubstituted arylene (e.g., C₆-C₁₀, C₁₀, or phenylene), or substituted or unsubstituted heteroarylene (e.g., 5 to 10 membered, 5 to 9 membered, or 5 to 6 membered), a peptide linker, or a cleavable linker.

For the PreQ1 analog, PreQ1 derivative and DNA compounds of formulas I-VIII provided herein, in embodiments, L¹ is a bond, —O—, —S—, —NH—, —C(O)NH₂, —NHC(O)—, —C(O)O—, —OC(O)—, —S(O)₂NH₂, —NHS(O)₂—, substituted or unsubstituted alkylene (e.g., C₁-C₈, C₁-C₆, or C₁-C₄), substituted or unsubstituted heteroalkylene (e.g., 2 to 8 membered, 2 to 6 membered, or 2 to 4 membered), substituted or unsubstituted cycloalkylene (e.g., C₃-C₈, C₃-C₆, or C₅-C₆), substituted or unsubstituted heterocycloalkylene (e.g., 3 to 8 membered, 3 to 6 membered, or 5 to 6 membered), substituted or unsubstituted arylene (e.g., C₆-C₁₀, C₁₀, or phenylene), or substituted or unsubstituted heteroarylene (e.g., 5 to 10 membered, 5 to 9 membered, or 5 to 6 membered), a peptide linker, or a cleavable linker. In embodiments of the PreQ1 derivative, L¹ is —CH₂—NH—.

For the PreQ1 analog, PreQ1 derivative and DNA compounds of formulas I-VIII provided herein, in embodiments, L² is a bond, —O—, —S—, —NH—, —C(O)NH₂, —NHC(O)—, —C(O)O—, —OC(O)—, —S(O)₂NH₂, —NHS(O)₂—, substituted or unsubstituted alkylene (e.g., C₁-C₈, C₁-C₆, or C₁-C₄), substituted or unsubstituted heteroalkylene (e.g., 2 to 8 membered, 2 to 6 membered, or 2 to 4 membered), substituted or unsubstituted cycloalkylene (e.g., C₃-C₈, C₃-C₆, or C₅-C₆), substituted or unsubstituted heterocycloalkylene (e.g., 3 to 8 membered, 3 to 6 membered, or 5 to 6 membered), substituted or unsubstituted arylene (e.g., C₆-C₁₀, C₁₀, or phenylene), or substituted or unsubstituted heteroarylene (e.g., 5 to 10 membered, 5 to 9 membered, or 5 to 6 membered), a peptide linker, or a cleavable linker.

For the PreQ1 analog, PreQ1 derivative and DNA compounds of formulas I-VIII provided herein, in embodiments, R³ is a detectable moiety, a biomolecular moiety, a therapeutic moiety, hydrogen, halogen, —CX^{3.1}₃, —CHX^{3.1}₂, —CH₂X^{3.1}, —CN, —SOₙ₁R^{3A}, —SOᵥ₁NR^{3B}R^{3C}, —NHNR^{3B}R^{3C}, —ONR^{3B}R^{3C}, —NHC(O)NHNR^{3B}R^{3C}, —NHC(O)NR^{3B}R^{3C}, —N(O)ₘ₁, —NR^{3B}R^{3C}, —C(O)R^{3D}—C(O)OR^{3D}, —C(O)NR^{3B}R^{3C}, —OR^{3A}, —NR^{3B}SO₂R^{3A}, —NR^{3B}C(O)R^{3D}. —NR^{3B}C(O)OR^{3D}, —NR^{3B}OR^{3D}, —OCX^{3.1}₃, —OCHX^{3.1}₂, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered).

For the PreQ1 analog, PreQ1 derivative and DNA compounds of formulas I-VIII provided herein, in embodiments, R^{3A}, R^{3B}, R^{3C} and R^{3D} are independently hydrogen, halogen, —CF₃, —CCl₃, —CBr₃, —
CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —
OCHI₂, —OCH₂Cl, —OCH₂Br, —OCH₂I, —OCH₂F, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered); R^{3B} and R^{3C} substituents bonded to the same nitrogen atom may optionally be joined to form a substituted or unsubstituted heterocycloalkyl (e.g., 3 to 8 membered. 3 to 6 membered, or 5 to 6 membered) or substituted or unsubstituted heteroaryl (e.g., 5 to 10 membered, 5 to 9 membered, or 5 to 6 membered).

For the PreQ1 analog, PreQ1 derivative and DNA compounds of formulas I-VIII provided herein, in embodiments, X^{3.1} is independently-Cl, —Br, —I or —F.

In embodiments, a substituted L¹ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹ is substituted with a plurality of groups selected from substituent groups, size-limited substituent groups, and lower substituent groups; each substituent group, size-limited substituent group, and/or lower substituent group may optionally be different. In embodiments, when L¹ is substituted, it is substituted with at least one substituent group. In embodiments, when L¹ is substituted, it is substituted with at least one size-limited substituent group. In embodiments, when L¹ is substituted, it is substituted with at least one lower substituent group.

In embodiments, L¹ is a bond, unsubstituted C₂-C₁₂ alkylene, or unsubstituted 2 to 12 membered heteroalkylene. In embodiments, L¹ is a bond. In embodiments, L¹ is an unsubstituted C₂-C₁₂ alkylene. In embodiments, L¹ is an unsubstituted 2 to 12 membered heteroalkylene.

In embodiments, L¹ is substituted or unsubstituted C₁-C₃ alkylene. In embodiments, L¹ is substituted or unsubstituted methylene. In embodiments, L¹ is substituted or unsubstituted C₁-C₆ alkylene, or substituted or unsubstituted 2 to 6 membered heteroalkylene. In embodiments, L¹ is substituted or unsubstituted C₁-C₃ alkylene, or substituted or unsubstituted 2 to 3 membered heteroalkylene.

In embodiments, L¹ is substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered). In embodiments, L¹ is substituted (e.g., substituted with a substituent group, a size-limited substituent group, or lower substituent group) or unsubstituted alkylene, substituted (e.g., substituted with a substituent group, a size-limited substituent group, or lower substituent group) or unsubstituted heteroalkylene, substituted (e.g., substituted with a substituent group, a size-limited substituent group, or lower substituent group) or unsubstituted cycloalkylene, substituted (e.g., substituted with a substituent group, a size-limited substituent group, or lower substituent group) or unsubstituted heterocycloalkylene, substituted (e.g., substituted with a substituent group, a size-limited substituent group, or lower substituent group) or unsubstituted arylene, or substituted (e.g., substituted with a substituent group, a size-limited substituent group, or lower substituent group) or unsubstituted heteroarylene. In embodiments, L¹ is unsubstituted alkylene, unsubstituted heteroalkylene, unsubstituted cycloalkylene, unsubstituted heterocycloalkylene, unsubstituted arylene, or unsubstituted heteroarylene. In embodiments, L¹ is unsubstituted alkylene (e.g., C₁-C₆ alkylene). In embodiments. L¹ is a bond. In embodiments, L¹ is a peptide linker. In embodiments, L¹ is a cleavable linker.

In embodiments, L¹ is a
bond, —C(O)—, —C(O)O—, —OC(O)—, —O—, —S—, —NH—, —C(O)NH—, —NHC(O)—, —NHC(O)O—, —OC(O)NH—, —NHC(O)NH—, —NHC(NH)NH—, —S(O)₂—, —NHS(O)₂—,
—S(O)₂NH—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), substituted or unsubstituted heteroarylene (e.g., 5 to 10 membered, 5 to 9 membered, or 5 to 6 membered), a peptide linker, or a cleavable linker.

In embodiments, L¹ is a bond. In embodiments, L¹ is —C(O)—. In embodiments, L¹ is —C(O)O—. In embodiments, L¹ is —OC(O)—. In embodiments, L¹ is —O—. In embodiments, L¹ is —S—. In embodiments, L¹ is —NH—. In embodiments, L¹ is —C(O)NH—. In embodiments, L¹ is —NHC(O)—. In embodiments, L¹ is —NHC(O)O—. In embodiments, L¹ is —OC(O)NH—. In embodiments, L¹ is —NHC(O)NH—. In embodiments, L¹ is —NHC(NH)NH—. In embodiments, L¹ is —S(O)₂—. In embodiments, L¹ is —NHS(O)₂—. In embodiments, L¹ is —S(O)₂NH—. In embodiments, L¹ is substituted or unsubstituted alkylene. In embodiments, L¹ is unsubstituted C₁-C₁₀alkylene. In embodiments, L¹ is unsubstituted methylene. In embodiments, L¹ is unsubstituted ethylene. In embodiments, L¹ is unsubstituted propylene. In embodiments, L¹ is unsubstituted n-propylene. In embodiments, L¹ is unsubstituted butylene. In embodiments, L¹ is unsubstituted n-butylene. In embodiments, L¹ is substituted or unsubstituted heteroalkylene. In embodiments, L¹ is substituted or unsubstituted 2 to 16 membered heteroalkylene. In embodiments, L¹ is substituted or unsubstituted 2 to 10 membered heteroalkylene. In embodiments, L¹ is substituted (e.g., oxo-substituted) 2 to 16 membered heteroalkylene. In embodiments, L¹ is substituted (e.g., oxo-substituted) 2 to 10 membered heteroalkylene. In embodiments, L¹ is -(unsubstituted C₁-C₁₀ alkylene)-NHC(O)—. In embodiments, L¹ is —(CH₂)—NHC(O)—. In embodiments, L¹ is —(CH₂)₂—NHC(O)—. In embodiments, L¹ is —(CH₂)₃—NHC(O)—. In embodiments, L¹ is —(CH₂)₄—NHC(O)—. In embodiments, L¹ is —(CH₂)₅—NHC(O)—. In embodiments, L¹ is —(CH₂)₆—NHC(O)—. In embodiments, L¹ is —(CH₂)₇—NHC(O)—. In embodiments, L¹ is —(CH₂)₈—NHC(O)—. In embodiments, L¹ is —(CH₂)₉—NHC(O)—. In embodiments, L¹ is —(CH₂)₁₀—NHC(O)—. In embodiments, L¹ is -(unsubstituted C₁-C₁₀ alkylene)-NHC(O)O—. In embodiments, L¹ is
—(CH₂)—NHC(O)O—. In embodiments, L¹ is —(CH₂)₂—NHC(O)O—. In embodiments, L¹ is
—(CH₂)₃—NHC(O)O—. In embodiments, L¹ is —(CH₂)₄—NHC(O)O—. In embodiments, L¹ is
—(CH₂)₅—NHC(O)O—. In embodiments, L¹ is —(CH₂)₆—NHC(O)O—. In embodiments, L¹ is
—(CH₂)₇—NHC(O)O—. In embodiments, L¹ is —(CH₂)₈—NHC(O)O—. In embodiments, L¹ is
—(CH₂)₉—NHC(O)O—. In embodiments, L¹ is —(CH₂)₁₀—NHC(O)O—. In embodiments, L¹ is
-(unsubstituted C₁-C₁₀ alkylene)-NHC(O)O-(unsubstituted C₁-C₁₀ alkylene)-. In embodiments, L¹ is —(CH₂)—NHC(O)O—C(CH₃)—. In embodiments, L¹ is —(CH₂)₂—NHC(O)O—C(CH₃)—. In embodiments, L¹ is —(CH₂)₃—NHC(O)O—C(CH₃)—. In embodiments, L¹ is
—(CH₂)₄—NHC(O)O—C(CH₃)—. In embodiments, L¹ is —(CH₂)₅—NHC(O)O—C(CH₃)—. In embodiments, L¹ is —(CH₂)₆—NHC(O)O—C(CH₃)—. In embodiments, L¹ is —(CH₂)₇—NHC(O)O—C(CH₃)—. In embodiments, L¹ is —(CH₂)₈—NHC(O)O—C(CH₃)—. In embodiments, L¹ is —(CH₂)₉—NHC(O)O—C(CH₃)—. In embodiments, L¹ is —(CH₂)₁₀—NHC(O)O—C(CH₃)—.

In embodiments, a substituted L² (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L² is substituted with a plurality of groups selected from substituent groups, size-limited substituent groups, and lower substituent groups; each substituent group, size-limited substituent group, and/or lower substituent group may optionally be different. In embodiments, when L² is substituted, it is substituted with at least one substituent group. In embodiments, when L² is substituted, it is substituted with at least one size-limited substituent group. In embodiments, when L² is substituted, it is substituted with at least one lower substituent group.

In embodiments, L² is a bond, unsubstituted C₂-C₁₂ alkylene, or unsubstituted 2 to 12 membered heteroalkylene. In embodiments, L² is a bond. In embodiments, L² is an unsubstituted C₂-C₁₂ alkylene. In embodiments, L² is an unsubstituted 2 to 12 membered heteroalkylene.

In embodiments, L² is substituted or unsubstituted C₁-C₃ alkylene. In embodiments, L² is substituted or unsubstituted methylene. In embodiments, L² is substituted or unsubstituted C₁-C₆ alkylene, or substituted or unsubstituted 2 to 6 membered heteroalkylene. In embodiments, L² is substituted or unsubstituted C₁-C₃ alkylene, or substituted or unsubstituted 2 to 3 membered heteroalkylene.

In embodiments, L² is substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered). In embodiments, L² is substituted (e.g., substituted with a substituent group, a size-limited substituent group, or lower substituent group) or unsubstituted alkylene, substituted (e.g., substituted with a substituent group, a size-limited substituent group, or lower substituent group) or unsubstituted heteroalkylene, substituted (e.g., substituted with a substituent group, a size-limited substituent group, or lower substituent group) or unsubstituted cycloalkylene, substituted (e.g., substituted with a substituent group, a size-limited substituent group, or lower substituent group) or unsubstituted heterocycloalkylene, substituted (e.g., substituted with a substituent group, a size-limited substituent group, or lower substituent group) or unsubstituted arylene, or substituted (e.g., substituted with a substituent group, a size-limited substituent group, or lower substituent group) or unsubstituted heteroarylene. In embodiments, L² is unsubstituted alkylene, unsubstituted heteroalkylene, unsubstituted cycloalkylene, unsubstituted heterocycloalkylene, unsubstituted arylene, or unsubstituted heteroarylene. In embodiments, L² is unsubstituted alkylene (e.g., C₁-C₆ alkylene). In embodiments, L² is a bond. In embodiments, L² is a peptide linker. In embodiments, L² is a cleavable linker.

In embodiments, L² is a
bond, —C(O)—, —C(O)O—, —OC(O)—, —O—, —S—, —NH—, —C(O)NH—,
—NHC(O)—, —NHC(O)O—, —OC(O)NH—, —NHC(O)NH—, —NHC(NH)NH—, —S(O)₂—, —NHS(O)₂—,
—S(O)₂NH—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), substituted or unsubstituted heteroarylene (e.g., 5 to 10 membered, 5 to 9 membered, or 5 to 6 membered), a peptide linker, or a cleavable linker.

In embodiments, L² is a bond. In embodiments, L² is —C(O)—. In embodiments, L² is —C(O)O—. In embodiments, L² is —OC(O)—. In embodiments, L² is —O—. In embodiments, L² is —S—. In embodiments, L² is —NH—. In embodiments, L² is —C(O)NH—. In embodiments, L² is —NHC(O)—. In embodiments, L² is —NHC(O)O—. In embodiments, L² is —OC(O)NH—. In embodiments, L² is —NHC(O)NH—. In embodiments, L² is —NHC(NH)NH—. In embodiments, L² is —S(O)₂—. In embodiments, L² is —NHS(O)₂—. In embodiments, L² is —S(O)₂NH—. In embodiments, L² is substituted or unsubstituted alkylene. In embodiments, L² is unsubstituted C₁-C₁₀alkylene. In embodiments, L² is unsubstituted methylene. In embodiments, L² is unsubstituted ethylene. In embodiments, L² is unsubstituted propylene. In embodiments, L² is unsubstituted n-propylene. In embodiments, L² is unsubstituted butylene. In embodiments, L² is unsubstituted n-butylene. In embodiments, L² is substituted or unsubstituted heteroalkylene. In embodiments, L² is substituted or unsubstituted 2 to 16 membered heteroalkylene. In embodiments, L² is substituted or unsubstituted 2 to 10 membered heteroalkylene. In embodiments, L² is substituted (e.g., oxo-substituted) 2 to 16 membered heteroalkylene. In embodiments, L² is substituted (e.g., oxo-substituted) 2 to 10 membered heteroalkylene. In embodiments, L² is -(unsubstituted C₁-C₁₀ alkylene)-NHC(O)—. In embodiments, L² is —(CH₂)—NHC(O)—. In embodiments, L² is —(CH₂)₂—NHC(O)—. In embodiments, L² is —(CH₂)₃—NHC(O)—. In embodiments, L² is —(CH₂)₄—NHC(O)—. In embodiments, L² is —(CH₂)₅—NHC(O)—. In embodiments, L² is —(CH₂)₆—NHC(O)—. In embodiments, L² is —(CH₂)₇—NHC(O)—. In embodiments, L² is —(CH₂)₈—NHC(O)—. In embodiments, L² is —(CH₂)₉—NHC(O)—. In embodiments, L² is —(CH₂)₁₀—NHC(O)—. In embodiments, L² is -(unsubstituted C₁-C₁₀ alkylene)-NHC(O)O—. In embodiments, L² is
—(CH₂)—NHC(O)O—. In embodiments, L² is —(CH₂)₂—NHC(O)O—. In embodiments, L² is
—(CH₂)₃—NHC(O)O—. In embodiments, L² is —(CH₂)₄—NHC(O)O—. In embodiments, L² is
—(CH₂)₅—NHC(O)O—. In embodiments, L² is —(CH₂)₆—NHC(O)O—. In embodiments, L² is
—(CH₂)₇—NHC(O)O—. In embodiments, L² is —(CH₂)₈—NHC(O)O—. In embodiments, L² is
—(CH₂)₉—NHC(O)O—. In embodiments, L² is —(CH₂)₁₀—NHC(O)O—. In embodiments, L² is
-(unsubstituted C₁-C₁₀ alkylene)-NHC(O)O-(unsubstituted C₁-C₁₀ alkylene)-. In embodiments, L² is —(CH₂)—NHC(O)O—C(CH₃)—. In embodiments, L² is —(CH₂)₂—NHC(O)O—C(CH₃)—. In embodiments, L² is —(CH₂)₃—NHC(O)O—C(CH₃)—. In embodiments, L² is
—(CH₂)₄—NHC(O)O—C(CH₃)—. In embodiments, L² is —(CH₂)₅—NHC(O)O—C(CH₃)—. In embodiments, L² is —(CH₂)₆—NHC(O)O—C(CH₃)—. In embodiments, L² is —(CH₂)₇—NHC(O)O—C(CH₃)—. In embodiments, L² is —(CH₂)₅—NHC(O)O—C(CH₃)—. In embodiments, L² is —(CH₂)₉—NHC(O)O—C(CH₃)—. In embodiments, L² is —(CH₂)₁₀—NHC(O)O—C(CH₃)—.

In embodiments, L¹ is —CH₂NH— and L² is —NHCH₂—. In embodiments, L¹ is —(CH₂)ₙ— and L² is —NHCH₂—, wherein n is an integer from 1 to 20. In embodiments, n is 1. In embodiments, n is 2. In embodiments, n is 3. In embodiments, n is 4. In embodiments, n is 5. In embodiments, n is 6. In embodiments, n is 7. In embodiments, n is 8. In embodiments, n is 9. In embodiments, n is 10. In embodiments, n is 11. In embodiments, n is 12. In embodiments, n is 13. In embodiments, n is 14. In embodiments, n is 15. In embodiments, n is 16. In embodiments, n is 17. In embodiments, n is 18. In embodiments, n is 19. In embodiments, n is 20.

In embodiments, -L¹-L²-R³ is

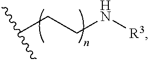

wherein n is an integer from 1 to 10. In embodiments, n is 1. In embodiments, n is 2. In embodiments, n is 3. In embodiments, n is 4. In embodiments, n is 5. In embodiments, n is 6. In embodiments, n is 7. In embodiments, n is 8. In embodiments, n is 9. In embodiments, n is 10.

In embodiments, -L¹-L²-R³ is,

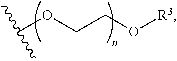

wherein n is an integer from 1 to 20. In embodiments, n is 1. In embodiments, n is 2. In embodiments, n is 3. In embodiments, n is 4. In embodiments, n is 5. In embodiments, n is 6. In embodiments, n is 7. In embodiments, n is 8. In embodiments, n is 9. In embodiments, n is 10. In embodiments, n is 11. In embodiments, n is 12. In embodiments, n is 13. In embodiments, n is 14. In embodiments, n is 15. In embodiments, n is 16. In embodiments, n is 17. In embodiments, n is 18. In embodiments, n is 19. In embodiments, n is 20.

In embodiments, L¹ is a bond or unsubstituted 2 to 12 membered heteroalkylene. In embodiments, L² is a bond or unsubstituted 2 to 12 membered heteroalkylene.

In embodiments, L¹ or L² includes a photo-cleavable site. As used herein, “photo-cleavable site” refers to a moiety which includes one or more covalent bonds that can be broken upon exposure to a specific wavelength or range of wavelengths. A photo-cleavable site allows release of the functional moiety R³ (e.g. therapeutic moiety) upon exposure of the DNA molecule to a specific wavelength. For example, photoactive compounds (e.g. coumarin) can be cleaved by visible light, thereby minimizing photo-toxicity when achieving cleavage of the linker. In another example, multiplexing of DNA molecules can be achieved by selecting L¹ or L² linkers that can be cleaved at different wavelengths. In embodiments, L¹ includes a photo-cleavable site. In embodiments, L² includes a photo-cleavable site. In embodiments, the photo-cleavable site is a monovalent form of an ortho-nitrobenzene, or derivative thereof. In embodiments, the photo-cleavable site is a divalent form of an ortho-nitrobenzene, or derivative thereof. In embodiments, the photo-cleavable site is

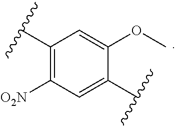 [SMILES: *c1cc([N+](=O)[O-])c(*)cc1OC]

In embodiments, the photo-cleavable site is a monovalent form of a coumarin, or derivative thereof. In embodiments, the photo-cleavable site is

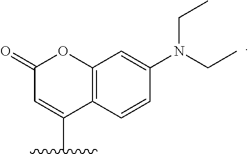 [SMILES: *c1cc(=O)oc2cc(N(CC)CC)ccc12]

In embodiments, the photo-cleavable site is a divalent form of a coumarin, or a derivative thereof. In embodiments, the photo-cleavable site is

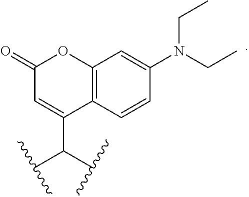 [SMILES: *C(*)c1cc(=O)oc2cc(N(CC)CC)ccc12]

In embodiments, the photo-cleavable site is a monovalent form of a salicyl-alcohol, or derivative thereof. In embodiments, the photo-cleavable site is a divalent form of a salicyl-alcohol, or derivative thereof.

In embodiments, L¹ or L² comprises a peptide linker. As used herein, a “peptide linker” refers to a covalent linker including 3 or more amino acid residues or derivatives thereof. In embodiments, L¹ includes a peptide linker. In embodiments, L² includes a peptide linker. In embodiments, the peptide linker is about 3 to about 60 amino acid residues in length. In embodiments, the peptide linker is about 6 to about 60 amino acid residues in length. In embodiments, the peptide linker is about 9 to about 60 amino acid residues in length. In embodiments, the peptide linker is about 12 to about 60 amino acid residues in length. In embodiments, the peptide linker is about 15 to about 60 amino acid residues in length. In embodiments, the peptide linker is about 18 to about 60 amino acid residues in length. In embodiments, the peptide linker is about 21 to about 60 amino acid residues in length. In embodiments, the peptide linker is about 24 to about 60 amino acid residues in length. In embodiments, the peptide linker is about 27 to about 60 amino acid residues in length. In embodiments, the peptide linker is about 30 to about 60 amino acid residues in length. In embodiments, the peptide linker is about 33 to about 60 amino acid residues in length. In embodiments, the peptide linker is about 36 to about 60 amino acid residues in length. In embodiments, the peptide linker is about 39 to about 60 amino acid residues in length. In embodiments, the peptide linker is about 42 to about 60 amino acid residues in length. In embodiments, the peptide linker is about 45 to about 60 amino acid residues in length. In embodiments, the peptide linker is about 48 to about 60 amino acid residues in length. In embodiments, the peptide linker is about 51 to about 60 amino acid residues in length. In embodiments, the peptide linker is about 54 to about 60 amino acid residues in length. In embodiments, the peptide linker is about 57 to about 60 amino acid residues in length. In embodiments, the peptide linker is about 3 to about 57 amino acid residues in length. In embodiments, the peptide linker is about 3 to about 54 amino acid residues in length. In embodiments, the peptide linker is about 3 to about 51 amino acid residues in length. In embodiments, the peptide linker is about 3 to about 48 amino acid residues in length. In embodiments, the peptide linker is about 3 to about 45 amino acid residues in length. In embodiments, the peptide linker is about 3 to about 42 amino acid residues in length. In embodiments, the peptide linker is about 3 to about 39 amino acid residues in length. In embodiments, the peptide linker is about 3 to about 36 amino acid residues in length. In embodiments, the peptide linker is about 3 to about 33 amino acid residues in length. In embodiments, the peptide linker is about 3 to about 30 amino acid residues in length. In embodiments, the peptide linker is about 3 to about 27 amino acid residues in length. In embodiments, the peptide linker is about 3 to about 24 amino acid residues in length. In embodiments, the peptide linker is about 3 to about 21 amino acid residues in length. In embodiments, the peptide linker is about 3 to about 18 amino acid residues in length. In embodiments, the peptide linker is about 3 to about 15 amino acid residues in length. In embodiments, the peptide linker is about 3 to about 12 amino acid residues in length. In embodiments, the peptide linker is about 3 to about 9 amino acid residues in length. In embodiments, the peptide linker is about 3 to about 6 amino acid residues in length. In embodiments, the peptide linker is 3 amino acid residues, 6 amino acid residues, 9 amino acid residues, 12 amino acid residues, 15 amino acid residues, 18 amino acid residues, 21 amino acid residues, 24 amino acid residues, 27 amino acid residues, 30 amino acid residues, 33 amino acid residues, 36 amino acid residues, 39 amino acid residues, 42 amino acid residues, 45 amino acid residues, 48 amino acid residues, 51 amino acid residues, 54 amino acid residues, 57 amino acid residues, or 60 amino acid residues in length.

In embodiments, peptide linker comprises a protease cleavable site. A “protease cleavable site” or “protease cleavage site” refers to a covalent bond (e.g. peptide bond) between amino acid residues in a peptide (e.g. peptide linker) that is broken upon binding of a protease to a protease recognition site. For example, the protease cleavable site may allow cleavage of the linker specifically at a tumor site (e.g. through a tumor specific protease). In embodiments, the protease cleavable site is a matrix metalloprotease (MMP) cleavage site, a metalloprotease domain-containing (ADAM) metalloprotease cleavage site, a lysosomal cathepsin cleavage site (ALAL), or a legumain endopeptidase cleavage site (TANL). In embodiments, the protease cleavable site is a MMP cleavage site. In embodiments, the protease cleavable site is an ADAM metalloprotease cleavage site. In embodiments, the protease cleavable site is an ALAL cleavage site. In embodiments, the protease cleavable site is a TANL cleavage site.

Further exemplary cleavage sites include the cleavage site of ABHD12, ADAM12, ABHD12B, ABHD13, ABHD17A, ADAM19, ADAM20, ADAM21, ADAM28, ADAM30, ADAM33, ADAM8, ABHD17A, ADAMDEC1, ADAMTS1, ADAMTS10, ADAMTS12, ADAMTS13, ADAMTS14, ADAMTS15, ADAMTS16, ADAMTS17, ADAMTS18, ADAMTS19, ADAMTS2, ADAMTS20, ADAMTS3, ADAMTS4, ABHD17B, ADAMTS5, ADAMTS6, ADAMTS7, ADAMTS8, ADAMTS9, ADAMTSL1, ADAMTSL2, ADAMTSL3, ABHD17C, ADAMTSL5, ASTL, BMP1, CELA1, CELA2A, CELA2B, CELA3A, CELA3B, ADAM10, ADAM15, ADAM17. ADAM9, ADAMTS4, CTSE, CTSF, ADAMTSL4, CMA1, CTRB1, CTRC, CTSO, CTRl, CTSA, CTSW, CTSB, CTSC, CTSD, ESP1, CTSG, CTSH, GZMA, GZMB, GZMH, CTSK, GZMM, CTSL, CTSS, CTSV, CTSZ, HTRA4, KLK10, KLK11, KLK13, KLK14, KLK2, KLK4, DPP4, KLK6, KLK7, KLKB1, ECE1, ECE2, ECEL1, MASP2, MEP1A, MEP1B, ELANE, FAP, GZMA, MMP11, GZMK, HGFAC, HPN, HTRA1, MMP11, MMP16, MMP17, MMP19, HTRA2, MMP20, MMP21, HTRA3, HTRA4, KEL, MMP23B, MMP24, MMP25, MMP26, MMP27, MMP28, KLK5, MMP3, MMP7, MMP8, MMP9, LGMN, LNPEP, MASP1, PAPPA, PAPPA2, PCSK1, NAPSA, PCSK5, PCSK6, MME, MMP1, MMP10, PLAT, PLAU, PLG, PRSS1, PRSS12, PRSS2, PRSS21, PRSS3, PRSS33, PRSS4, PRSS55, PRSS57, MMP12, PRSS8, PRSS9, PRTN3, MMP13, MMP14, ST14, TMPRSS10, TMPRSS11A, TMPRSS11D, TMPRSS11E, TMPRSS11F, TMPRSS12, TMPRSS13, MMP15, TMPRSS15, MMP2, TMPRSS2, TMPRSS3, TMPRSS4, TMPRSS5, TMPRSS6, TMPRSS7, TMPRSS9, NRDC, OVCH1, PAMR1, PCSK3, PHEX, TINAG, TPSAB1, TPSD1, or TPSG1.

In embodiments, L¹ includes a pH sensitive cleavable site. A “pH sensitive cleavable site” refers to a covalent bond that can be cleaved (e.g. self-cleaving, cleaved by a protease) at a specific pH or within a range of pHs. In embodiments, the pH sensitive cleavable site is a divalent form of a phosphoramidyl hydroxypropylglycine. In embodiments, the pH sensitive cleavable site is a divalent form of a phosphoramidyl homoserine. In embodiments, the pH sensitive cleavable site is a divalent form of a phosphoramidyl serine. Examples of pH sensitive cleavable linkers are described in detail in Leriche, G., Chisholm, L., Wagner, A; Cleavable linkers in chemical biology, Bioorganic & Medicinal Chemistry, Volume 20, Issue 2, 2012, Pages 571-582, ISSN 0968-0896, https://doi.org/10.1016/j.bmc.2011.07.048.; which is incorporated herein in its entirety and for all purposes. In embodiments, the pH cleavable site is cleaved at pH 6.5 or lower. In embodiments, the pH cleavable site is cleaved at pH 6 or lower. In embodiments, the pH cleavable site is cleaved at pH 5.5 or lower. In embodiments, the pH cleavable site is cleaved at pH 5 or lower. In embodiments, the pH cleavable site is cleaved at pH 4.5 or lower. In embodiments, the pH cleavable site is cleaved at pH 4 or lower. In embodiments, the pH cleavable site is cleaved at pH 3.5 or lower. In embodiments, the pH cleavable site is cleaved at pH 3 or lower.

In embodiments, a substituted R³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³ is substituted with a plurality of groups selected from substituent groups, size-limited substituent groups, and lower substituent groups; each substituent group, size-limited substituent group, and/or lower substituent group may optionally be different. In embodiments, when R³ is substituted, it is substituted with at least one substituent group. In embodiments, when R³ is substituted, it is substituted with at least one size-limited substituent group. In embodiments, when R³ is substituted, it is substituted with at least one lower substituent group.

In embodiments, a substituted R^{3A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3A} is substituted with a plurality of groups selected from substituent groups, size-limited substituent groups, and lower substituent groups; each substituent group, size-limited substituent group, and/or lower substituent group may optionally be different. In embodiments, when R^{3A} is substituted, it is substituted with at least one substituent group. In embodiments, when R^{3A} is substituted, it is substituted with at least one size-limited substituent group. In embodiments, when R^{3A} is substituted, it is substituted with at least one lower substituent group.

In embodiments, a substituted R^{3B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3B} is substituted with a plurality of groups selected from substituent groups, size-limited substituent groups, and lower substituent groups; each substituent group, size-limited substituent group, and/or lower substituent group may optionally be different. In embodiments, when R^{3B} is substituted, it is substituted with at least one substituent group. In embodiments, when R^{3B} is substituted, it is substituted with at least one size-limited substituent group. In embodiments, when R^{3B} is substituted, it is substituted with at least one lower substituent group.

In embodiments, a substituted R^{3C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3C} is substituted with a plurality of groups selected from substituent groups, size-limited substituent groups, and lower substituent groups; each substituent group, size-limited substituent group, and/or lower substituent group may optionally be different. In embodiments, when R^{3C} is substituted, it is substituted with at least one substituent group. In embodiments, when R^{3C} is substituted, it is substituted with at least one size-limited substituent group. In embodiments, when R^{3C} is substituted, it is substituted with at least one lower substituent group.

In embodiments, a substituted ring formed when R^{3B} and R^{3C} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{3B} and R^{3C} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the ring formed when R^{3B} and R^{3C} substituents bonded to the same nitrogen atom are joined is substituted, it is substituted with at least one substituent group. In embodiments, when the ring formed when R^{3B} and R^{3C} substituents bonded to the same nitrogen atom are joined is substituted, it is substituted with at least one size-limited substituent group. In embodiments, when the ring formed when R^{3B} and R^{3C} substituents bonded to the same nitrogen atom are joined is substituted, it is substituted with at least one lower substituent group.

In embodiments, a substituted R^{3D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3D} is substituted with a plurality of groups selected from substituent groups, size-limited substituent groups, and lower substituent groups; each substituent group, size-limited substituent group, and/or lower substituent group may optionally be different. In embodiments, when R^{3D} is substituted, it is substituted with at least one substituent group. In embodiments, when R^{3D} is substituted, it is substituted with at least one size-limited substituent group. In embodiments, when R^{3D} is substituted, it is substituted with at least one lower substituent group.

In embodiments, R³ is a detectable moiety, a biomolecular moiety, a therapeutic moiety, hydrogen, halogen, —CF₃, —CCl₃, —CBr₃, —
CI₃, —CHCl₂, —CHBr₂, —CHF₂, —CHI₂, —CH₂Cl, —CH₂Br,
—CH₂F, —CH₂I, —CN, —OH. —NH₂, —COOH, —CONH₂, —NO₂, —SH, —SO₃H, —S04H, —SO₂NH₂, —NHNH₂, —ONH₂, —NHC(O)NHNH₂, —NHC(O)NH₂, —NHSO₂H, —NHC(O)H, —NHC(O)—OH,
—NHOH, —OCF₃, —OCCl₃, —OCBr₃, —OCI₃, —OCHF₂, —OCHCl₂, —OCHBr₂, —OCHI₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R³ is a detectable moiety. As described above, a detectable moiety is a composition detectable by spectroscopic, photochemical, biochemical, immunochemical, chemical, or other physical means. In embodiments, the detectable moiety is a fluorescent moiety. In instances, the detectable moiety allows identification or quantification of the DNA compound provided herein including embodiments thereof. In other instances, the detectable moiety allows for identification of an oligonucleotide targeted by the DNA compound provided herein including embodiments thereof. For example, the DNA compound may be a probe for a target oligonuleotide that is at least partially complementary to a sequence within the DNA compound provided herein. Thus, when R³ is a detectable moiety, the DNA compound may be a probe (e.g. a fluorscence in situ hybridication (FISH) probe, a hybridization probe, etc.). In embodiments, R³ is a monovalent form of TAMRA. In embodiments, R³ is a monovalent form of Cy3. In embodiments, R³ is a monovalent form of Cy5. In embodiments, R³ is a monovalent form of Cy7. In embodiments, R³ is a monovalent form of AlexaFluor 647. In embodiments, R³ is a monovalent form of AlexaFluor 488. In embodiments, R³ is a monovalent form of AMCA. In embodiments, R³ is a monovalent form of DAPI. In embodiments, R³ is a monovalent form of fluorescein. In embodiments, R³ is a monovalent form of rhodamine. In embodiments, R³ is a monovalent form of Texas Red. In embodiments, R³ is a monovalent form of TRITC. In embodiments, R³ is a monovalent form of benzyl guanine.

In embodiments, R³ is a biomolecular moiety. “Biomolecule” is used in its customary sense and refers to a molecule naturally produced by a cell or organism, or a derivative of a molecule naturally produced by a cell or organism. Biomolecules include proteins, carbohydrates, lipids, nucleic acids, and natural products. In embodiments, the biomolecular moiety is a peptide nucleic acid, sugar, peptide, antibody or fragment thereof, lipid, or affinity ligand (e.g. biotin, hapten, etc.). In embodiments, the biomolecule includes one of a pair of binding partners, e.g., biotin, streptavidin. In embodiments, R³ is biotin (see, for example, FIG. 2A). In embodiments, R³ is a monovalent form of biotin. In embodiments, a“biomolecular moiety” refers to the monovalent form of a biomolecule.

In embodiments, R³ is a therapeutic moiety. The term “therapeutic moiety” refers to a composition which has a physiological effect (e.g., beneficial effect, is useful for treating a subject) when introduced into or to a subject (e.g., in or on the body of a subject or patient). A therapeutic moiety is a radical of a drug. A therapeutic moiety may be useful for treating or preventing a condition or disease. In embodiments, the therapeutic moiety is an antibody or fragment thereof.

In embodiments, when R³ is substituted, R³ is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R³, R^{3.1}, R^{3.2}, and R^{3.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R³, R^{3.1}, R^{3.2}, and R^{3.3}, respectively.

In embodiments, when R^{3A} is substituted, R^{3A} is substituted with one or more first substituent groups denoted by R^{3A.1} as explained in the definitions section above in the description of “first substituent group(s)”. In embodiments, when an R^{3A.1} substituent group is substituted, the R^{3A.1} substituent group is substituted with one or more second substituent groups denoted by R^{3A.2} as explained in the definitions section above in the description of “first substituent group(s)”. In embodiments, when an R^{3A.2} substituent group is substituted, the R^{3A.2} substituent group is substituted with one or more third substituent groups denoted by R^{3A.3} as explained in the definitions section above in the description of “first substituent group(s)”. In the above embodiments, R^{3A}, R^{3A.1}, R^{3A.2}, and R^{3A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3A}, R^{3A.1}, R^{3A.2}, and R^{3A.3}, respectively.

In embodiments, when R^{3B} is substituted, R^{3B} is substituted with one or more first substituent groups denoted by R^{3B.1} as explained in the definitions section above in the description of “first substituent group(s)”. In embodiments, when an R^{3B.1} substituent group is substituted, the R^{3B.1} substituent group is substituted with one or more second substituent groups denoted by R^{3B.2} as explained in the definitions section above in the description of “first substituent group(s)”. In embodiments, when an R^{3B.2} substituent group is substituted, the R^{3B.2} substituent group is substituted with one or more third substituent groups denoted by R^{3B.3} as explained in the definitions section above in the description of “first substituent group(s)”. In the above embodiments, R^{3B}, R^{3B.1}, R^{3B.2}, and R^{3B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3B}, R^{3B.1}, R^{3B.2}, and R^{3B.3} respectively.

In embodiments, when R^{3C} is substituted, R^{3C} is substituted with one or more first substituent groups denoted by R^{3C.1} as explained in the definitions section above in the description of “first substituent group(s)”. In embodiments, when an R^{3C.1} substituent group is substituted, the R^{3C.1} substituent group is substituted with one or more second substituent groups denoted by R^{3C.2} as explained in the definitions section above in the description of “first substituent group(s)”. In embodiments, when an R^{3C.2} substituent group is substituted, the R^{3C.2} substituent group is substituted with one or more third substituent groups denoted by R^{3C.3} as explained in the definitions section above in the description of “first substituent group(s)”. In the above embodiments, R^{3C}, R^{3C.1}, R^{3C.2}, and R^{3C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3}, correspond to R^{3C}, R^{3C.1}, R^{3C.2}, and R^{3C.3}, respectively.

In embodiments, when R^{3B} and R^{3C} substituents bonded to the same nitrogen atom are optionally joined to form a moiety that is substituted (e.g., a substituted heterocycloalkyl or substituted heteroaryl), the moiety is substituted with one or more first substituent groups denoted by R^{3B.1} as explained in the definitions section above in the description of “first substituent group(s)”. In embodiments, when an R^{3B.1} substituent group is substituted, the R^{3B.1} substituent group is substituted with one or more second substituent groups denoted by R^{3B.2} as explained in the definitions section above in the description of “first substituent group(s)”. In embodiments, when an R^{3B.2} substituent group is substituted, the R^{3B.2} substituent group is substituted with one or more third substituent groups denoted by R^{3B.3} as explained in the definitions section above in the description of “first substituent group(s)”. In the above embodiments, R^{3B.1}, R^{3B.2} and R^{3B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3B.1}, R^{3B.2} and R^{3B.3}, respectively.

In embodiments, when R^{3B} and R^{3C} substituents bonded to the same nitrogen atom are optionally joined to form a moiety that is substituted (e.g., a substituted heterocycloalkyl or substituted heteroaryl), the moiety is substituted with one or more first substituent groups denoted by R^{3C.1} as explained in the definitions section above in the description of “first substituent group(s)”. In embodiments, when an R^{3C.1} substituent group is substituted, the R^{3C.1} substituent group is substituted with one or more second substituent groups denoted by R^{3C.2} as explained in the definitions section above in the description of “first substituent group(s)”. In embodiments, when an R^{3C.2} substituent group is substituted, the R^{3C.2} substituent group is substituted with one or more third substituent groups denoted by R^{3C.3} as explained in the definitions section above in the description of “first substituent group(s)”. In the above embodiments, R^{3C.1}, R^{3C.2} and R^{3C.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3C.1}, R^{3C.2} and R^{3C.3} respectively.

In embodiments, when R^{3D} is substituted, R^{3D} is substituted with one or more first substituent groups denoted by R^{3D.1} as explained in the definitions section above in the description of “first substituent group(s)”. In embodiments, when an R^{3D.1} substituent group is substituted, the R^{3D.1} substituent group is substituted with one or more second substituent groups denoted by R^{3D.2} as explained in the definitions section above in the description of “first substituent group(s)”. In embodiments, when an R^{3D.2} substituent group is substituted, the R^{3D.2} substituent group is substituted with one or more third substituent groups denoted by R^{3D.3} as explained in the definitions section above in the description of “first substituent group(s)”. In the above embodiments, R^{3D}, R^{3D.1}, R^{3D.2}, and R^{3D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3}, correspond to R^{3D}, R^{3D.1}, R^{3D.2}, and R^{3D.3}, respectively.

In embodiments, when L¹ is substituted, L¹ is substituted with one or more first substituent groups denoted by R^{L1.1} as explained in the definitions section above in the description of “first substituent group(s)”. In embodiments, when an R^{L1.1} substituent group is substituted, the R^{L1.1} substituent group is substituted with one or more second substituent groups denoted by R^{L1.2} as explained in the definitions section above in the description of “first substituent group(s)”. In embodiments, when an R^{L1.2} substituent group is substituted, the R^{L1.2} substituent group is substituted with one or more third substituent groups denoted by R^{L1.3} as explained in the definitions section above in the description of “first substituent group(s)”. In the above embodiments, L¹, R^{L1.1}, R^{L1.2}, and R^{L1.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹, R^{L1.1}, R^{L1.2}, and R^{L1.3} respectively.

In embodiments, when L² is substituted, L² is substituted with one or more first substituent groups denoted by R^{L2.1} as explained in the definitions section above in the description of “first substituent group(s)”. In embodiments, when an R^{L2.1} substituent group is substituted, the R^{L2.1} substituent group is substituted with one or more second substituent groups denoted by R^{L2.2} as explained in the definitions section above in the description of “first substituent group(s)”. In embodiments, when an R^{L2.2} substituent group is substituted, the R^{L2.2} substituent group is substituted with one or more third substituent groups denoted by R^{L2.3} as explained in the definitions section above in the description of “first substituent group(s)”. In the above embodiments, L², R^{L2.1}, R^{L2.2}, and R^{L2.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L², R^{L2.1}, R^{L2.2}, and R^{L2.3}, respectively.

The DNA compounds (e.g. the DNA compounds of formula I-VIII) provided herein including embodiments thereof are useful for detecting or quantifying a target oligonucleotide. The target oligonucleotide may be, for example, a gene, a gene fragment, an RNA target, a sequence including a single nucleotide polymorphism, etc. Thus, a DNA compound may include a sequence that is at least partially complementary to a target oligonucleotide, thereby allowing the DNA compound to hybridize to the target oligonucleotide. The DNA compounds may be used for multiplex assays, e.g. to detect and/or quantify multiple target oligonucleotides. The DNA compounds therefore may be used for multiplex in situ hybridization (M-FISH) and multiplex PCR. Thus, in an aspect, a composition including a plurality of DNA compounds provided herein including embodiments thereof is provided. In embodiments, each of the plurality of DNA compounds includes a sequence at least partially complementary to a target oligonucleotide. In embodiments, the plurality of DNA compounds are probes or primers. In embodiments, the plurality of DNA compounds are probes. In embodiments, the plurality of DNA compounds are primers. For the compositions provided herein, in embodiments, each of the DNA compounds in the plurality of compounds includes a different R³, wherein each of the R³ is a detectable moiety. For example, each R³ may be a fluorescent moiety having a different excitation and emission wavelength, thereby allowing detection and/or quantification of each oligonucleotide target.

### Methods for Modifying DNA

Provided herein are methods for modifying DNA molecules with preQ1 analogs or derivatives decorated with functional moieties (e.g. detectable moieties, therapeutic moieties, etc.). Specifically, methods provided herein include replacement of a guanine nucleobase within a hairpin structure in a DNA molecule with a preQ1 analog or derivative. Use of linkers, for example, polyethylene glycol (PEG) linkers, or alkyl, alkenyl, or alkynyl linkers can allow for attachment of a functional moiety to a preQ1 analog or derivative, thereby allowing incorporation of the functional moiety into the DNA molecule, as described in detail throughout the specification. Methods described herein may be achieved by using bacterial (E.coli) tRNA Guanine Transglycosylase (TGT). TGT is known to incorporate preQ1 into tRNA. Applicant has discovered that, surprisingly, DNA (instead of RNA) can be modified by the enzyme, and furthermore, the DNA may be functionalized by using a preQ1 analog or derivative. Applicant has further shown that PreQ1 analogs and derivatives as provided herein can be into DNA molecules with comparable efficiency to that of the natural substrate preQ1. Thus, the methods provided herein allow for incorporation of bioorthogonal ligation partners, fluorescent moieties, therapeutic moieties, and affinity labels (such as biotin), into a DNA molecule via linkage to a preQ1 analog or derivative.

In an aspect is provided a method of modifying a DNA molecule, the method including contacting the DNA molecule with a tRNA-guanine transglycosylase (TGT) enzyme and a PreQ1 analog, wherein the DNA molecule includes a guanine nucleobase within a loop portion of a hairpin in the DNA molecule, and wherein the PreQ1 analog has the formula:

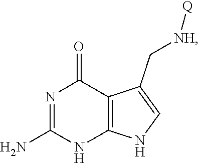

wherein Q is not hydrogen.

In another aspect is provided a method of modifying a DNA molecule, the method including contacting the DNA molecule with a tRNA-guanine transglycosylase (TGT) enzyme and a PreQ1 derivative, wherein the DNA molecule includes a guanine nucleobase within a loop portion of a hairpin in the DNA molecule, and wherein the PreQ1 derivative has the formula

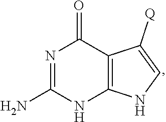

wherein Q is not hydrogen. For the PreQ1 derivative above, Q is as set forth herein, including all embodiments thereof.

In embodiments, the guanine nucleobase is in a YYGYYYY (SEQ ID NO: 103) sequence within the loop portion of the hairpin in the DNA molecule. In embodiments, the sequence within the loop portion of the hairpin is YTGTYYY (SEQ ID NO:104), YTGTCCY (SEQ ID NO:105), YTGTYCC (SEQ ID NO:106), YUGUYYY (SEQ ID NO:107), YUGTYYY (SEQ ID NO:108), or YTGUYYY (SEQ ID NO:109). In embodiments, the sequence within the loop portion of the hairpin is YTGTYYY (SEQ ID NO:104). In embodiments, the sequence within the loop portion of the hairpin is YTGTCCY (SEQ ID NO:105). In embodiments, the sequence within the loop portion of the hairpin is YTGTYCC (SEQ ID NO:106). In embodiments, the sequence within the loop portion of the hairpin is YUGUYYY (SEQ ID NO:107). In embodiments, the sequence within the loop portion of the hairpin is YUGTYYY (SEQ ID NO:108), or YTGUYYY (SEQ ID NO:109). In embodiments, the sequence within the loop portion of the hairpin is YTGUYYY (SEQ ID NO:109).

In embodiments, the sequence within the loop portion of the hairpin is YTGTCCY (SEQ ID NO:105) or YTGTYCC (SEQ ID NO:106). In embodiments, Y includes a pyrimidine nucleobase. In embodiments, Y is cytosine or thymine.

In embodiments, the DNA molecule is between about 15 nucleotides to about 30,000 nucleotides in length. In embodiments, the DNA molecule is between about 600 nucleotides to about 30,000 nucleotides in length. In embodiments, the DNA molecule is between about 1200 nucleotides to about 30,000 nucleotides in length. In embodiments, the DNA molecule is between about 1800 nucleotides to about 30,000 nucleotides in length. In embodiments, the DNA molecule is between about 2400 nucleotides to about 30,000 nucleotides in length. In embodiments, the DNA molecule is between about 3000 nucleotides to about 30,000 nucleotides in length. In embodiments, the DNA molecule is between about 3600 nucleotides to about 30,000 nucleotides in length. In embodiments, the DNA molecule is between about 4200 nucleotides to about 30,000 nucleotides in length. In embodiments, the DNA molecule is between about 4800 nucleotides to about 30,000 nucleotides in length. In embodiments, the DNA molecule is between about 5400 nucleotides to about 30,000 nucleotides in length. In embodiments, the DNA molecule is between about 6000 nucleotides to about 30,000 nucleotides in length. In embodiments, the DNA molecule is between about 6600 nucleotides to about 30,000 nucleotides in length. In embodiments, the DNA molecule is between about 7200 nucleotides to about 30,000 nucleotides in length. In embodiments, the DNA molecule is between about 7800 nucleotides to about 30,000 nucleotides in length. In embodiments, the DNA molecule is between about 8400 nucleotides to about 30,000 nucleotides in length. In embodiments, the DNA molecule is between about 9000 nucleotides to about 30,000 nucleotides in length. In embodiments, the DNA molecule is between about 9600 nucleotides to about 30,000 nucleotides in length. In embodiments, the DNA molecule is between about 10,200 nucleotides to about 30,000 nucleotides in length. In embodiments, the DNA molecule is between about 10,800 nucleotides to about 30,000 nucleotides in length. In embodiments, the DNA molecule is between about 11,400 nucleotides to about 30,000 nucleotides in length. In embodiments, the DNA molecule is between about 12,000 nucleotides to about 30,000 nucleotides in length. In embodiments, the DNA molecule is between about 12,600 nucleotides to about 30,000 nucleotides in length. In embodiments, the DNA molecule is between about 13,200 nucleotides to about 30,000 nucleotides in length. In embodiments, the DNA molecule is between about 13,800 nucleotides to about 30,000 nucleotides in length. In embodiments, the DNA molecule is between about 14,400 nucleotides to about 30,000 nucleotides in length. In embodiments, the DNA molecule is between about 15,000 nucleotides to about 30,000 nucleotides in length. In embodiments, the DNA molecule is between about 15,600 nucleotides to about 30,000 nucleotides in length. In embodiments, the DNA molecule is between about 16,200 nucleotides to about 30,000 nucleotides in length. In embodiments, the DNA molecule is between about 16,800 nucleotides to about 30,000 nucleotides in length. In embodiments, the DNA molecule is between about 17,400 nucleotides to about 30,000 nucleotides in length. In embodiments, the DNA molecule is between about 18,000 nucleotides to about 30,000 nucleotides in length. In embodiments, the DNA molecule is between about 18,600 nucleotides to about 30,000 nucleotides in length. In embodiments, the DNA molecule is between about 19,200 nucleotides to about 30,000 nucleotides in length. In embodiments, the DNA molecule is between about 19,800 nucleotides to about 30,000 nucleotides in length. In embodiments, the DNA molecule is between about 20,400 nucleotides to about 30,000 nucleotides in length. In embodiments, the DNA molecule is between about 21,000 nucleotides to about 30,000 nucleotides in length. In embodiments, the DNA molecule is between about 21,600 nucleotides to about 30,000 nucleotides in length. In embodiments, the DNA molecule is between about 22,200 nucleotides to about 30,000 nucleotides in length. In embodiments, the DNA molecule is between about 22,800 nucleotides to about 30,000 nucleotides in length. In embodiments, the DNA molecule is between about 23,400 nucleotides to about 30,000 nucleotides in length. In embodiments, the DNA molecule is between about 24,000 nucleotides to about 30,000 nucleotides in length. In embodiments, the DNA molecule is between about 24,600 nucleotides to about 30,000 nucleotides in length. In embodiments, the DNA molecule is between about 25,200 nucleotides to about 30,000 nucleotides in length. In embodiments, the DNA molecule is between about 25,800 nucleotides to about 30,000 nucleotides in length. In embodiments, the DNA molecule is between about 25,400 nucleotides to about 30,000 nucleotides in length. In embodiments, the DNA molecule is between about 27,000 nucleotides to about 30,000 nucleotides in length. In embodiments, the DNA molecule is between about 27,600 nucleotides to about 30,000 nucleotides in length. In embodiments, the DNA molecule is between about 28,200 nucleotides to about 30,000 nucleotides in length. In embodiments, the DNA molecule is between about 28,800 nucleotides to about 30,000 nucleotides in length. In embodiments, the DNA molecule is between about 29,400 nucleotides to about 30,000 nucleotides in length.

In embodiments, the DNA molecule is between about 15 nucleotides to about 29,400 nucleotides in length. In embodiments, the DNA molecule is between about 15 nucleotides to about 28,800 nucleotides in length. In embodiments, the DNA molecule is between about 15 nucleotides to about 28,200 nucleotides in length. In embodiments, the DNA molecule is between about 15 nucleotides to about 27,600 nucleotides in length. In embodiments, the DNA molecule is between about 15 nucleotides to about 27,000 nucleotides in length. In embodiments, the DNA molecule is between about 15 nucleotides to about 26,400 nucleotides in length. In embodiments, the DNA molecule is between about 15 nucleotides to about 25,800 nucleotides in length. In embodiments, the DNA molecule is between about 15 nucleotides to about 25,200 nucleotides in length. In embodiments, the DNA molecule is between about 15 nucleotides to about 24,600 nucleotides in length. In embodiments, the DNA molecule is between about 15 nucleotides to about 24,000 nucleotides in length. In embodiments, the DNA molecule is between about 15 nucleotides to about 30,000 nucleotides in length. In embodiments, the DNA molecule is between about 15 nucleotides to about 23,400 nucleotides in length. In embodiments, the DNA molecule is between about 15 nucleotides to about 22,800 nucleotides in length. In embodiments, the DNA molecule is between about 15 nucleotides to about 22,200 nucleotides in length. In embodiments, the DNA molecule is between about 15 nucleotides to about 21,600 nucleotides in length. In embodiments, the DNA molecule is between about 15 nucleotides to about 21,000 nucleotides in length. In embodiments, the DNA molecule is between about 15 nucleotides to about 20,400 nucleotides in length. In embodiments, the DNA molecule is between about 15 nucleotides to about 19,800 nucleotides in length. In embodiments, the DNA molecule is between about 15 nucleotides to about 19,200 nucleotides in length. In embodiments, the DNA molecule is between about 15 nucleotides to about 18,600 nucleotides in length. In embodiments, the DNA molecule is between about 15 nucleotides to about 18,000 nucleotides in length. In embodiments, the DNA molecule is between about 15 nucleotides to about 17,400 nucleotides in length. In embodiments, the DNA molecule is between about 15 nucleotides to about 16,800 nucleotides in length. In embodiments, the DNA molecule is between about 15 nucleotides to about 16,200 nucleotides in length. In embodiments, the DNA molecule is between about 15 nucleotides to about 15,600 nucleotides in length. In embodiments, the DNA molecule is between about 15 nucleotides to about 15,000 nucleotides in length. In embodiments, the DNA molecule is between about 15 nucleotides to about 14,400 nucleotides in length. In embodiments, the DNA molecule is between about 15 nucleotides to about 13,800 nucleotides in length. In embodiments, the DNA molecule is between about 15 nucleotides to about 12,600 nucleotides in length. In embodiments, the DNA molecule is between about 15 nucleotides to about 12,000 nucleotides in length. In embodiments, the DNA molecule is between about 15 nucleotides to about 11,400 nucleotides in length. In embodiments, the DNA molecule is between about 15 nucleotides to about 10,800 nucleotides in length. In embodiments, the DNA molecule is between about 15 nucleotides to about 10,200 nucleotides in length. In embodiments, the DNA molecule is between about 15 nucleotides to about 9,600 nucleotides in length. In embodiments, the DNA molecule is between about 15 nucleotides to about 9000 nucleotides in length. In embodiments, the DNA molecule is between about 15 nucleotides to about 8400 nucleotides in length. In embodiments, the DNA molecule is between about 15 nucleotides to about 7800 nucleotides in length. In embodiments, the DNA molecule is between about 15 nucleotides to about 7200 nucleotides in length. In embodiments, the DNA molecule is between about 15 nucleotides to about 6600 nucleotides in length. In embodiments, the DNA molecule is between about 15 nucleotides to about 6000 nucleotides in length. In embodiments, the DNA molecule is between about 15 nucleotides to about 5400 nucleotides in length. In embodiments, the DNA molecule is between about 15 nucleotides to about 4800 nucleotides in length. In embodiments, the DNA molecule is between about 15 nucleotides to about 4200 nucleotides in length. In embodiments, the DNA molecule is between about 15 nucleotides to about 3600 nucleotides in length. In embodiments, the DNA molecule is between about 15 nucleotides to about 3000 nucleotides in length. In embodiments, the DNA molecule is between about 15 nucleotides to about 2400 nucleotides in length. In embodiments, the DNA molecule is between about 15 nucleotides to about 1800 nucleotides in length. In embodiments, the DNA molecule is between about 15 nucleotides to about 1200 nucleotides in length. In embodiments, the DNA molecule is between about 15 nucleotides to about 600 nucleotides in length. In embodiments, the DNA molecule is about 15, 600, 1200, 1800, 2400, 3000, 3600, 4200, 4800, 5400, 6000, 6600, 7200, 7800, 8400, 9000, 9600, 10,200, 10,800, 11,400, 12,000, 12,600, 13,200, 13,800, 14,400, 15,000, 15,600, 16,200, 16,800, 17,400, 18,000, 18,600, 19,200, 19,800, 20,400, 21,000, 21,600, 22,200, 22,800, 23,400, 24,000, 24,600, 25,200, 25,800, 26,400, 27,000, 27,600, 28,200, 28,800, 29,400, or 30,000 nucleotides in length.

In embodiments, the DNA molecule is between about 15 nucleotides to about 10,000 nucleotides in length. In embodiments, the DNA molecule is about 200 nucleotides to about 10,000 nucleotides in length. In embodiments, the DNA molecule is about 400 nucleotides to about 10,000 nucleotides in length. In embodiments, the DNA molecule is about 600 nucleotides to about 10,000 nucleotides in length. In embodiments, the DNA molecule is about 800 nucleotides to about 10,000 nucleotides in length. In embodiments, the DNA molecule is about 1,000 nucleotides to about 10,000 nucleotides in length. In embodiments, the DNA molecule is about 1,200 nucleotides to about 10,00 nucleotides in length. In embodiments, the DNA molecule is about 1,400 nucleotides to about 10,000 nucleotides in length. In embodiments, the DNA molecule is about 1,600 nucleotides to about 10,000 nucleotides in length. In embodiments, the DNA molecule is about 1,800 nucleotides to about 10,000 nucleotides in length. In embodiments, the DNA molecule is about 2,000 nucleotides to about 10,000 nucleotides in length. In embodiments, the DNA molecule is about 2,200 nucleotides to about 10,000 nucleotides in length. In embodiments, the DNA molecule is about 2,400 nucleotides to about 10,000 nucleotides in length. In embodiments, the DNA molecule is about 2,600 nucleotides to about 10,000 nucleotides in length. In embodiments, the DNA molecule is about 2,800 nucleotides to about 10,000 nucleotides in length. In embodiments, the DNA molecule is about 3,000 nucleotides to about 10,000 nucleotides in length. In embodiments, the DNA molecule is about 3,200 nucleotides to about 10,000 nucleotides in length. In embodiments, the DNA molecule is about 3,400 nucleotides to about 10,000 nucleotides in length. In embodiments, the DNA molecule is about 3,600 nucleotides to about 10,000 nucleotides in length. In embodiments, the DNA molecule is about 3,800 nucleotides to about 10,000 nucleotides in length. In embodiments, the DNA molecule is about 4,000 nucleotides to about 10,000 nucleotides in length. In embodiments, the DNA molecule is about 4,200 nucleotides to about 10,000 nucleotides in length. In embodiments, the DNA molecule is about 4,400 nucleotides to about 10,000 nucleotides in length. In embodiments, the DNA molecule is about 4,600 nucleotides to about 10,000 nucleotides in length. In embodiments, the DNA molecule is about 4,800 nucleotides to about 10,000 nucleotides in length. In embodiments, the DNA molecule is about 5,000 nucleotides to about 10,000 nucleotides in length. In embodiments, the DNA molecule is about 5,200 nucleotides to about 10,000 nucleotides in length. In embodiments, the DNA molecule is about 5,400 nucleotides to about 10,000 nucleotides in length. In embodiments, the DNA molecule is about 5,600 nucleotides to about 10,000 nucleotides in length. In embodiments, the DNA molecule is about 5,800 nucleotides to about 10,000 nucleotides in length. In embodiments, the DNA molecule is about 6,000 nucleotides to about 10,000 nucleotides in length. In embodiments, the DNA molecule is about 6,200 nucleotides to about 10,000 nucleotides in length. In embodiments, the DNA molecule is about 6,400 nucleotides to about 10,000 nucleotides in length. In embodiments, the DNA molecule is about 6,600 nucleotides to about 10,000 nucleotides in length. In embodiments, the DNA molecule is about 6,800 nucleotides to about 10,000 nucleotides in length. In embodiments, the DNA molecule is about 7,000 nucleotides to about 10,000 nucleotides in length. In embodiments, the DNA molecule is about 7,200 nucleotides to about 10,000 nucleotides in length. In embodiments, the DNA molecule is about 7,400 nucleotides to about 10,000 nucleotides in length. In embodiments, the DNA molecule is about 7,600 nucleotides to about 10,000 nucleotides in length. In embodiments, the DNA molecule is about 7,800 nucleotides to about 10,000 nucleotides in length. In embodiments, the DNA molecule is about 8,000 nucleotides to about 10,000 nucleotides in length. In embodiments, the DNA molecule is about 8,200 nucleotides to about 10,000 nucleotides in length. In embodiments, the DNA molecule is about 8,400 nucleotides to about 10,000 nucleotides in length. In embodiments, the DNA molecule is about 8,600 nucleotides to about 10,000 nucleotides in length. In embodiments, the DNA molecule is about 8,800 nucleotides to about 10,000 nucleotides in length. In embodiments, the DNA molecule is about 9,000 nucleotides to about 10,000 nucleotides in length. In embodiments, the DNA molecule is about 9,200 nucleotides to about 10,000 nucleotides in length. In embodiments, the DNA molecule is about 9,400 nucleotides to about 10,000 nucleotides in length. In embodiments, the DNA molecule is about 9,600 nucleotides to about 10,000 nucleotides in length. In embodiments, the DNA molecule is about 9,800 nucleotides to about 10,000 nucleotides in length.

In embodiments, the DNA molecule is between about 15 nucleotides to about 9,800 nucleotides in length. In embodiments, the DNA molecule is between about 15 nucleotides to about 9,600 nucleotides in length. In embodiments, the DNA molecule is between about 15 nucleotides to about 9,400 nucleotides in length. In embodiments, the DNA molecule is between about 15 nucleotides to about 9,200 nucleotides in length. In embodiments, the DNA molecule is between about 15 nucleotides to about 9,000 nucleotides in length. In embodiments, the DNA molecule is between about 15 nucleotides to about 8,800 nucleotides in length. In embodiments, the DNA molecule is between about 15 nucleotides to about 8,600 nucleotides in length. In embodiments, the DNA molecule is between about 15 nucleotides to about 8,400 nucleotides in length. In embodiments, the DNA molecule is between about 15 nucleotides to about 8,200 nucleotides in length. In embodiments, the DNA molecule is between about 15 nucleotides to about 8,000 nucleotides in length. In embodiments, the DNA molecule is between about 15 nucleotides to about 7,800 nucleotides in length. In embodiments, the DNA molecule is between about 15 nucleotides to about 7,600 nucleotides in length. In embodiments, the DNA molecule is between about 15 nucleotides to about 7,400 nucleotides in length. In embodiments, the DNA molecule is between about 15 nucleotides to about 7,200 nucleotides in length. In embodiments, the DNA molecule is between about 15 nucleotides to about 7,000 nucleotides in length. In embodiments, the DNA molecule is between about 15 nucleotides to about 6,800 nucleotides in length. In embodiments, the DNA molecule is between about 15 nucleotides to about 6,600 nucleotides in length. In embodiments, the DNA molecule is between about 15 nucleotides to about 6,400 nucleotides in length. In embodiments, the DNA molecule is between about 15 nucleotides to about 6,200 nucleotides in length. In embodiments, the DNA molecule is between about 15 nucleotides to about 6,000 nucleotides in length. In embodiments, the DNA molecule is between about 15 nucleotides to about 5,800 nucleotides in length. In embodiments, the DNA molecule is between about 15 nucleotides to about 5,600 nucleotides in length. In embodiments, the DNA molecule is between about 15 nucleotides to about 5,400 nucleotides in length. In embodiments, the DNA molecule is between about 15 nucleotides to about 5,200 nucleotides in length. In embodiments, the DNA molecule is between about 15 nucleotides to about 5,000 nucleotides in length. In embodiments, the DNA molecule is between about 15 nucleotides to about 4,800 nucleotides in length. In embodiments, the DNA molecule is between about 15 nucleotides to about 4,600 nucleotides in length. In embodiments, the DNA molecule is between about 15 nucleotides to about 4,400 nucleotides in length. In embodiments, the DNA molecule is between about 15 nucleotides to about 4,200 nucleotides in length. In embodiments, the DNA molecule is between about 15 nucleotides to about 4,000 nucleotides in length. In embodiments, the DNA molecule is between about 15 nucleotides to about 3,800 nucleotides in length. In embodiments, the DNA molecule is between about 15 nucleotides to about 3,600 nucleotides in length. In embodiments, the DNA molecule is between about 15 nucleotides to about 3,400 nucleotides in length. In embodiments, the DNA molecule is between about 15 nucleotides to about 3,200 nucleotides in length. In embodiments, the DNA molecule is between about 15 nucleotides to about 2,000 nucleotides in length. In embodiments, the DNA molecule is between about 15 nucleotides to about 1,800 nucleotides in length. In embodiments, the DNA molecule is between about 15 nucleotides to about 1,600 nucleotides in length. In embodiments, the DNA molecule is between about 15 nucleotides to about 1,400 nucleotides in length. In embodiments, the DNA molecule is between about 15 nucleotides to about 1,200 nucleotides in length. In embodiments, the DNA molecule is between about 15 nucleotides to about 1,000 nucleotides in length. In embodiments, the DNA molecule is between about 15 nucleotides to about 800 nucleotides in length. In embodiments, the DNA molecule is between about 15 nucleotides to about 600 nucleotides in length. In embodiments, the DNA molecule is between about 15 nucleotides to about 400 nucleotides in length. In embodiments, the DNA molecule is between about 15 nucleotides to about 200 nucleotides in length. In embodiments, the DNA molecule is 15 nucleotides, 200 nucleotides in length, 400 nucleotides in length, 600 nucleotides in length, 800 nucleotides in length, 1,000 nucleotides in length, 1200 nucleotides in length, 1400 nucleotides in length, 1600 nucleotides in length, 1800 nucleotides in length, 2000 nucleotides in length, 2200 nucleotides in length, 2400 nucleotides in length, 2600 nucleotides in length, 2800 nucleotides in length, 3000 nucleotides in length, 3200 nucleotides in length, 3400 nucleotides in length, 3600 nucleotides in length, 3800 nucleotides in length, 4000 nucleotides in length, 4200 nucleotides in length, 4400 nucleotides in length, 4600 nucleotides in length, 4800 nucleotides in length, 5000 nucleotides in length, 5200 nucleotides in length, 5400 nucleotides in length, 5600 nucleotides in length, 5800 nucleotides in length, 6000 nucleotides in length, 6200 nucleotides in length, 6400 nucleotides in length, 6600 nucleotides in length, 6800 nucleotides in length, 7000 nucleotides in length, 7200 nucleotides in length, 7400 nucleotides in length, 7600 nucleotides in length, 7800 nucleotides in length, 8000 nucleotides in length, 8200 nucleotides in length, 8400 nucleotides in length, 8600 nucleotides in length, 8800 nucleotides in length, 9000 nucleotides in length, 9200 nucleotides in length, 9400 nucleotides in length, 9600 nucleotides in length, 9800 nucleotides in length, or 10,000 nucleotides in length.

In embodiments, the DNA molecule is between about 20 nucleotides to about 300 nucleotides in length. In embodiments, the DNA molecule is between about 40 nucleotides to about 300 nucleotides in length. In embodiments, the DNA molecule is between about 60 nucleotides to about 300 nucleotides in length. In embodiments, the DNA molecule is between about 80 nucleotides to about 300 nucleotides in length. In embodiments, the DNA molecule is between about 100 nucleotides to about 300 nucleotides in length. In embodiments, the DNA molecule is between about 120 nucleotides to about 300 nucleotides in length. In embodiments, the DNA molecule is between about 140 nucleotides to about 300 nucleotides in length. In embodiments, the DNA molecule is between about 160 nucleotides to about 300 nucleotides in length. In embodiments, the DNA molecule is between about 180 nucleotides to about 300 nucleotides in length. In embodiments, the DNA molecule is between about 200 nucleotides to about 300 nucleotides in length. In embodiments, the DNA molecule is between about 220 nucleotides to about 300 nucleotides in length. In embodiments, the DNA molecule is between about 240 nucleotides to about 300 nucleotides in length. In embodiments, the DNA molecule is between about 260 nucleotides to about 300 nucleotides in length. In embodiments, the DNA molecule is between about 280 nucleotides to about 300 nucleotides in length.

In embodiments, the DNA molecule is between about 20 nucleotides to about 280 nucleotides in length. In embodiments, the DNA molecule is between about 20 nucleotides to about 260 nucleotides in length. In embodiments, the DNA molecule is between about 20 nucleotides to about 240 nucleotides in length. In embodiments, the DNA molecule is between about 20 nucleotides to about 220 nucleotides in length. In embodiments, the DNA molecule is between about 20 nucleotides to about 200 nucleotides in length. In embodiments, the DNA molecule is between about 20 nucleotides to about 180 nucleotides in length. In embodiments, the DNA molecule is between about 20 nucleotides to about 160 nucleotides in length. In embodiments, the DNA molecule is between about 20 nucleotides to about 140 nucleotides in length. In embodiments, the DNA molecule is between about 20 nucleotides to about 120 nucleotides in length. In embodiments, the DNA molecule is between about 20 nucleotides to about 100 nucleotides in length. In embodiments, the DNA molecule is between about 20 nucleotides to about 80 nucleotides in length. In embodiments, the DNA molecule is between about 20 nucleotides to about 60 nucleotides in length. In embodiments, the DNA molecule is between about 20 nucleotides to about 40 nucleotides in length. In embodiments, the DNA molecule is 20 nucleotides, 40 nucleotides, 60 nucleotides, 80 nucleotides, 100 nucleotides, 120 nucleotides, 140 nucleotides, 160 nucleotides, 180 nucleotides, 200 nucleotides, 220 nucleotides, 240 nucleotides, 260 nucleotides, 280 nucleotides, or 300 nucleotides in length.

In embodiments, the DNA molecule is between about 5 nucleotides to about 50 nucleotides in length. In embodiments, the DNA molecule is between about 10 nucleotides to about 50 nucleotides in length. In embodiments, the DNA molecule is between about 15 nucleotides to about 50 nucleotides in length. In embodiments, the DNA molecule is between about 20 nucleotides to about 50 nucleotides in length. In embodiments, the DNA molecule is between about 25 nucleotides to about 50 nucleotides in length. In embodiments, the DNA molecule is between about 30 nucleotides to about 50 nucleotides in length. In embodiments, the DNA molecule is between about 35 nucleotides to about 50 nucleotides in length. In embodiments, the DNA molecule is between about 40 nucleotides to about 50 nucleotides in length. In embodiments, the DNA molecule is between about 55 nucleotides to about 50 nucleotides in length.

In embodiments, the DNA molecule is between about 5 nucleotides to about 45 nucleotides in length. In embodiments, the DNA molecule is between about 5 nucleotides to about 40 nucleotides in length. In embodiments, the DNA molecule is between about 5 nucleotides to about 35 nucleotides in length. In embodiments, the DNA molecule is between about 5 nucleotides to about 30 nucleotides in length. In embodiments, the DNA molecule is between about 5 nucleotides to about 25 nucleotides in length. In embodiments, the DNA molecule is between about 5 nucleotides to about 20 nucleotides in length. In embodiments, the DNA molecule is between about 5 nucleotides to about 15 nucleotides in length. In embodiments, the DNA molecule is between about 5 nucleotides to about 10 nucleotides in length. In embodiments, the DNA molecule is about 5, 10, 15, 20, 25, 30, 35, 40, 45 or 50 nucleotides in length.

In embodiments, the DNA molecule provided herein includes any one of SEQ ID NO:1-102. In embodiments, the DNA molecule includes the sequence of SEQ ID NO:1. In embodiments, the DNA molecule includes the sequence of SEQ ID NO:2. In embodiments, the DNA molecule includes the sequence of SEQ ID NO:3. In embodiments, the DNA molecule includes the sequence of SEQ ID NO:4. In embodiments, the DNA molecule includes the sequence of SEQ ID NO:5. In embodiments, the DNA molecule includes the sequence of SEQ ID NO: 6. In embodiments, the DNA molecule includes the sequence of SEQ ID NO: 7. In embodiments, the DNA molecule includes the sequence of SEQ ID NO: 8. In embodiments, the DNA molecule includes the sequence of SEQ ID NO:9. In embodiments, the DNA molecule includes the sequence of SEQ ID NO:10. In embodiments, the DNA molecule includes the sequence of SEQ ID NO:11. In embodiments, the DNA molecule includes the sequence of SEQ ID NO:12, In embodiments, the DNA molecule includes the sequence of SEQ ID NO:13. In embodiments, the DNA molecule includes the sequence of SEQ ID NO:14. In embodiments, the DNA molecule includes the sequence of SEQ ID NO:15. In embodiments, the DNA molecule includes the sequence of SEQ ID NO:16. In embodiments, the DNA molecule includes the sequence of SEQ ID NO:17. In embodiments, the DNA molecule includes the sequence of SEQ ID NO:18. In embodiments, the DNA molecule includes the sequence of SEQ ID NO:19. In embodiments, the DNA molecule includes the sequence of SEQ ID NO:20. In embodiments, the DNA molecule includes the sequence of SEQ ID NO:21. In embodiments, the DNA molecule includes the sequence of SEQ ID NO:22. In embodiments, the DNA molecule includes the sequence of SEQ ID NO:23. In embodiments, the DNA molecule includes the sequence of SEQ ID NO:24. In embodiments, the DNA molecule includes the sequence of SEQ ID NO:25. In embodiments, the DNA molecule includes the sequence of SEQ ID NO:26. In embodiments, the DNA molecule includes the sequence of SEQ ID NO:27. In embodiments, the DNA molecule includes the sequence of SEQ ID NO:28. In embodiments, the DNA molecule includes the sequence of SEQ ID NO:29. In embodiments, the DNA molecule includes the sequence of SEQ ID NO:30. In embodiments, the DNA molecule includes the sequence of SEQ ID NO:31. In embodiments, the DNA molecule includes the sequence of SEQ ID NO:32. In embodiments, the DNA molecule includes the sequence of SEQ ID NO:33. In embodiments, the DNA molecule includes the sequence of SEQ ID NO:34. In embodiments, the DNA molecule includes the sequence of SEQ ID NO:35. In embodiments, the DNA molecule includes the sequence of SEQ ID NO:36. In embodiments, the DNA molecule includes the sequence of SEQ ID NO:37. In embodiments, the DNA molecule includes the sequence of SEQ ID NO:38. In embodiments, the DNA molecule includes the sequence of SEQ ID NO:39. In embodiments, the DNA molecule includes the sequence of SEQ ID NO:40. In embodiments, the DNA molecule includes the sequence of SEQ ID NO:41. In embodiments, the DNA molecule includes the sequence of SEQ ID NO:42. In embodiments, the DNA molecule includes the sequence of SEQ ID NO:43. In embodiments, the DNA molecule includes the sequence of SEQ ID NO:44. In embodiments, the DNA molecule includes the sequence of SEQ ID NO:45. In embodiments, the DNA molecule includes the sequence of SEQ ID NO:46. In embodiments, the DNA molecule includes the sequence of SEQ ID NO:47. In embodiments, the DNA molecule includes the sequence of SEQ ID NO:48. In embodiments, the DNA molecule includes the sequence of SEQ ID NO:49. In embodiments, the DNA molecule includes the sequence of SEQ ID NO:50. In embodiments, the DNA molecule includes the sequence of SEQ ID NO:51. In embodiments, the DNA molecule includes the sequence of SEQ ID NO:52. In embodiments, the DNA molecule includes the sequence of SEQ ID NO:53. In embodiments, the DNA molecule includes the sequence of SEQ ID NO:54. In embodiments, the DNA molecule includes the sequence of SEQ ID NO:55. In embodiments, the DNA molecule includes the sequence of SEQ ID NO:56. In embodiments, the DNA molecule includes the sequence of SEQ ID NO:57. In embodiments, the DNA molecule includes the sequence of SEQ ID NO:58. In embodiments, the DNA molecule includes the sequence of SEQ ID NO:59. In embodiments, the DNA molecule includes the sequence of SEQ ID NO:60. In embodiments, the DNA molecule includes the sequence of SEQ ID NO:61. In embodiments, the DNA molecule includes the sequence of SEQ ID NO:62. In embodiments, the DNA molecule includes the sequence of SEQ ID NO:63. In embodiments, the DNA molecule includes the sequence of SEQ ID NO:64. In embodiments, the DNA molecule includes the sequence of SEQ ID NO:65, In embodiments, the DNA molecule includes the sequence of SEQ ID NO:66. In embodiments, the DNA molecule includes the sequence of SEQ ID NO:67. In embodiments, the DNA molecule includes the sequence of SEQ ID NO:68. In embodiments, the DNA molecule includes the sequence of SEQ ID NO:69. In embodiments, the DNA molecule includes the sequence of SEQ ID NO:70. In embodiments, the DNA molecule includes the sequence of SEQ ID NO:71. In embodiments, the DNA molecule includes the sequence of SEQ ID NO:72. In embodiments, the DNA molecule includes the sequence of SEQ ID NO:73. In embodiments, the DNA molecule includes the sequence of SEQ ID NO:74. In embodiments, the DNA molecule includes the sequence of SEQ ID NO:75. In embodiments, the DNA molecule includes the sequence of SEQ ID NO:76, In embodiments, the DNA molecule includes the sequence of SEQ ID NO:77. In embodiments, the DNA molecule includes the sequence of SEQ ID NO:78. In embodiments, the DNA molecule includes the sequence of SEQ ID NO:79. In embodiments, the DNA molecule includes the sequence of SEQ ID NO:80. In embodiments, the DNA molecule includes the sequence of SEQ ID NO:81. In embodiments, the DNA molecule includes the sequence of SEQ ID NO:82. In embodiments, the DNA molecule includes the sequence of SEQ ID NO:83. In embodiments, the DNA molecule includes the sequence of SEQ ID NO:84. In embodiments, the DNA molecule includes the sequence of SEQ ID NO:85. In embodiments, the DNA molecule includes the sequence of SEQ ID NO:86. In embodiments, the DNA molecule includes the sequence of SEQ ID NO:87. In embodiments, the DNA molecule includes the sequence of SEQ ID NO:88. In embodiments, the DNA molecule includes the sequence of SEQ ID NO:89. In embodiments, the DNA molecule includes the sequence of SEQ ID NO:90. In embodiments, the DNA molecule includes the sequence of SEQ ID NO:91. In embodiments, the DNA molecule includes the sequence of SEQ ID NO:92. In embodiments, the DNA molecule includes the sequence of SEQ ID NO:93. In embodiments, the DNA molecule includes the sequence of SEQ ID NO:94. In embodiments, the DNA molecule includes the sequence of SEQ ID NO:95. In embodiments, the DNA molecule includes the sequence of SEQ ID NO:96. In embodiments, the DNA molecule includes the sequence of SEQ ID NO:97, In embodiments, the DNA molecule includes the sequence of SEQ ID NO:98. In embodiments, the DNA molecule includes the sequence of SEQ ID NO:99. In embodiments, the DNA molecule includes the sequence of SEQ ID NO:100. In embodiments, the DNA molecule includes the sequence of SEQ ID NO:101. In embodiments, the DNA molecule includes the sequence of SEQ ID NO:102.

In embodiments, the DNA molecule provided herein is any one of SEQ ID NO:1-102. In embodiments, the DNA molecule is the sequence of SEQ ID NO:1. In embodiments, the DNA molecule is the sequence of SEQ ID NO:2. In embodiments, the DNA molecule is the sequence of SEQ ID NO:3. In embodiments, the DNA molecule is the sequence of SEQ ID NO:4. In embodiments, the DNA molecule is the sequence of SEQ ID NO:5. In embodiments, the DNA molecule is the sequence of SEQ ID NO:6. In embodiments, the DNA molecule is the sequence of SEQ ID NO:7. In embodiments, the DNA molecule is the sequence of SEQ ID NO:8. In embodiments, the DNA molecule is the sequence of SEQ ID NO:9. In embodiments, the DNA molecule is the sequence of SEQ ID NO:10. In embodiments, the DNA molecule is the sequence of SEQ ID NO:11. In embodiments, the DNA molecule is the sequence of SEQ ID NO:12. In embodiments, the DNA molecule is the sequence of SEQ ID NO:13. In embodiments, the DNA molecule is the sequence of SEQ ID NO:14. In embodiments, the DNA molecule is the sequence of SEQ ID NO:15. In embodiments, the DNA molecule is the sequence of SEQ ID NO:16. In embodiments, the DNA molecule is the sequence of SEQ ID NO:17. In embodiments, the DNA molecule is the sequence of SEQ ID NO:18. In embodiments, the DNA molecule is the sequence of SEQ ID NO: 19. In embodiments, the DNA molecule is the sequence of SEQ ID NO:20. In embodiments, the DNA molecule is the sequence of SEQ ID NO:21. In embodiments, the DNA molecule is the sequence of SEQ ID NO:22. In embodiments, the DNA molecule is the sequence of SEQ ID NO:23. In embodiments, the DNA molecule is the sequence of SEQ ID NO:24. In embodiments, the DNA molecule is the sequence of SEQ ID NO:25. In embodiments, the DNA molecule is the sequence of SEQ ID NO:26. In embodiments, the DNA molecule is the sequence of SEQ ID NO:27. In embodiments, the DNA molecule is the sequence of SEQ ID NO:28. In embodiments, the DNA molecule is the sequence of SEQ ID NO:29. In embodiments, the DNA molecule is the sequence of SEQ ID NO:30. In embodiments, the DNA molecule is the sequence of SEQ ID NO:31. In embodiments, the DNA molecule is the sequence of SEQ ID NO:32. In embodiments, the DNA molecule is the sequence of SEQ ID NO:33. In embodiments, the DNA molecule is the sequence of SEQ ID NO:34. In embodiments, the DNA molecule is the sequence of SEQ ID NO:35. In embodiments, the DNA molecule is the sequence of SEQ ID NO:36. In embodiments, the DNA molecule is the sequence of SEQ ID NO:37. In embodiments, the DNA molecule is the sequence of SEQ ID NO:38. In embodiments, the DNA molecule is the sequence of SEQ ID NO:39. In embodiments, the DNA molecule is the sequence of SEQ ID NO:40. In embodiments, the DNA molecule is the sequence of SEQ ID NO:41. In embodiments, the DNA molecule is the sequence of SEQ ID NO:42. In embodiments, the DNA molecule is the sequence of SEQ ID NO:43. In embodiments, the DNA molecule is the sequence of SEQ ID NO:44. In embodiments, the DNA molecule is the sequence of SEQ ID NO:45. In embodiments, the DNA molecule is the sequence of SEQ ID NO:46. In embodiments, the DNA molecule is the sequence of SEQ ID NO:47. In embodiments, the DNA molecule is the sequence of SEQ ID NO:48. In embodiments, the DNA molecule is the sequence of SEQ ID NO:49. In embodiments, the DNA molecule is the sequence of SEQ ID NO:50. In embodiments, the DNA molecule is the sequence of SEQ ID NO:51. In embodiments, the DNA molecule is the sequence of SEQ ID NO:52. In embodiments, the DNA molecule is the sequence of SEQ ID NO:53. In embodiments, the DNA molecule is the sequence of SEQ ID NO:54. In embodiments, the DNA molecule is the sequence of SEQ ID NO:55. In embodiments, the DNA molecule is the sequence of SEQ ID NO:56. In embodiments, the DNA molecule is the sequence of SEQ ID NO:57. In embodiments, the DNA molecule is the sequence of SEQ ID NO:58. In embodiments, the DNA molecule is the sequence of SEQ ID NO:59. In embodiments, the DNA molecule is the sequence of SEQ ID NO:60. In embodiments, the DNA molecule is the sequence of SEQ ID NO:61. In embodiments, the DNA molecule is the sequence of SEQ ID NO:62. In embodiments, the DNA molecule is the sequence of SEQ ID NO:63. In embodiments, the DNA molecule is the sequence of SEQ ID NO:64. In embodiments, the DNA molecule is the sequence of SEQ ID NO:65. In embodiments, the DNA molecule is the sequence of SEQ ID NO:66. In embodiments, the DNA molecule is the sequence of SEQ ID NO:67, In embodiments, the DNA molecule is the sequence of SEQ ID NO:68. In embodiments, the DNA molecule is the sequence of SEQ ID NO:69. In embodiments, the DNA molecule is the sequence of SEQ ID NO:70. In embodiments, the DNA molecule is the sequence of SEQ ID NO:71. In embodiments, the DNA molecule is the sequence of SEQ ID NO:72. In embodiments, the DNA molecule is the sequence of SEQ ID NO:73. In embodiments, the DNA molecule is the sequence of SEQ ID NO:74. In embodiments, the DNA molecule is the sequence of SEQ ID NO:75. In embodiments, the DNA molecule is the sequence of SEQ ID NO:76. In embodiments, the DNA molecule is the sequence of SEQ ID NO:77. In embodiments, the DNA molecule is the sequence of SEQ ID NO:78. In embodiments, the DNA molecule is the sequence of SEQ ID NO:79. In embodiments, the DNA molecule is the sequence of SEQ ID NO:80. In embodiments, the DNA molecule is the sequence of SEQ ID NO:81. In embodiments, the DNA molecule is the sequence of SEQ ID NO:82. In embodiments, the DNA molecule is the sequence of SEQ ID NO:83. In embodiments, the DNA molecule is the sequence of SEQ ID NO:84. In embodiments, the DNA molecule is the sequence of SEQ ID NO:85. In embodiments, the DNA molecule is the sequence of SEQ ID NO:86. In embodiments, the DNA molecule is the sequence of SEQ ID NO:87. In embodiments, the DNA molecule is the sequence of SEQ ID NO:88. In embodiments, the DNA molecule is the sequence of SEQ ID NO:89. In embodiments, the DNA molecule is the sequence of SEQ ID NO:90. In embodiments, the DNA molecule is the sequence of SEQ ID NO:91. In embodiments, the DNA molecule is the sequence of SEQ ID NO:92. In embodiments, the DNA molecule is the sequence of SEQ ID NO:93. In embodiments, the DNA molecule is the sequence of SEQ ID NO:94. In embodiments, the DNA molecule is the sequence of SEQ ID NO:95. In embodiments, the DNA molecule is the sequence of SEQ ID NO:96. In embodiments, the DNA molecule is the sequence of SEQ ID NO:97. In embodiments, the DNA molecule is the sequence of SEQ ID NO:98. In embodiments, the DNA molecule is the sequence of SEQ ID NO:99. In embodiments, the DNA molecule is the sequence of SEQ ID NO:100. In embodiments, the DNA molecule is the sequence of SEQ ID NO:101. In embodiments, the DNA molecule is the sequence of SEQ ID NO: 102.

In embodiments, the DNA molecule is a gene, a cDNA, a non-coding DNA, an extracellular DNA (eDNA), an antisense oligonucleotide, a barcode, a probe, or a primer. In embodiments, the DNA molecule is a gene. In embodiments, the DNA molecule is a cDNA. In embodiments, the DNA molecule is a a non-coding DNA. In embodiments, the DNA molecule is an extracellular DNA (eDNA). In embodiments, the DNA molecule is an antisense oligonucleotide. In embodiments, the DNA molecule is a barcode. In embodiments, the DNA molecule is a a probe. In embodiments, the DNA molecule is a primer.

“Non-coding DNA” is used according to its ordinary meaning in the art and refer to DNA sequences that do not encode protein sequences. In instances, non-coding DNA is transcribed into non-coding RNA (e.g. tRNA, miRNA, rRNA, regulatory RNAs, etc. A non-coding DNA may be a promoter sequence or a regulatory element. In instances, a non-coding DNA may be a telomere sequence or an intronic sequence.

A “barcode” refers to a DNA molecule or sequence within a DNA molecule that may be used to identify a cell or a subset of cells within a population with which the barcode is associated. For example, a barcode may be present in about one cell in a population of cells. Alternatively, a barcode may be present in a subset of cells within a population of cells, each cell of the subset may contain the same barcode, thereby allowing identification of the subset of cells with the population of cells.

An “antisense nucleic acid” or “antisense oligonucleotide” as referred to herein is a nucleic acid (e.g., DNA molecule) that is complementary to at least a portion of a specific target nucleic acid. In embodiments, the antisense nucleic acid is capable of reducing transcription of the target nucleic acid (e.g. mRNA from DNA), reducing the translation of the target nucleic acid (e.g. mRNA), altering transcript splicing (e.g. single stranded morpholino oligo), or interfering with the endogenous activity of the target nucleic acid. See, e.g., Weintraub, Scientific American, 262:40 (1990). Typically, synthetic antisense nucleic acids (e.g. oligonucleotides) are generally between 15 and 25 bases in length. Thus, antisense nucleic acids are capable of hybridizing to (e.g. selectively hybridizing to) a target nucleic acid. In embodiments, the antisense nucleic acid hybridizes to the target nucleic acid in vitro. In embodiments, the antisense nucleic acid hybridizes to the target nucleic acid in a cell. In embodiments, the antisense nucleic acid hybridizes to the target nucleic acid in an organism. In embodiments, the antisense nucleic acid hybridizes to the target nucleic acid under physiological conditions. Antisense nucleic acids may comprise naturally occurring nucleotides or modified nucleotides such as, e.g., phosphorothioate, methylphosphonate, and -anomeric sugar-phosphate, backbonemodified nucleotides. In the cell, the antisense nucleic acids hybridize to the corresponding RNA forming a double-stranded molecule. The antisense nucleic acids interfere with the endogenous behavior of the RNA and inhibit its function relative to the absence of the antisense nucleic acid. Furthermore, the double-stranded molecule may be degraded via the RNAi pathway. The use of antisense methods to inhibit the in vitro translation of genes is well known in the art (Marcus-Sakura, Anal. Biochem., 172:289, (1988)). Further, antisense molecules which bind directly to the DNA may be used. Antisense nucleic acids may be single or double stranded nucleic acids. Non-limiting examples of antisense nucleic acids include siRNAs (including their derivatives or pre-cursors, such as nucleotide analogs), short hairpin RNAs (shRNA), micro RNAs (miRNA), saRNAs (small activating RNAs) and small nucleolar RNAs (snoRNA) or certain of their derivatives or pre-cursors.

A “probe” or “DNA probe” refers to a DNA molecule including a single-stranded DNA sequence that is used to detect the presence of a target oligonucleotide by hybridization to a complementary nucleic acid sequence. The DNA probe typically includes a detectable moiety, for example, a fluorescent moiety. The DNA probe may be labeled with a radioisotope, epitope or biotin to allow for its detection. In embodiments, the DNA probe is cDNA, which can function as a hybridation probe for its complementary sequence. In embodiments, the DNA probe includes a short sequence (e.g. about 10 to about 40 nucleotides in length) that is complementary to its target sequence, thereby allowing detection of a single nucleotide polymorphism.

“Primer” refers to a DNA molecule including a single-stranded sequence that is at least partially complementary to a target sequence and is used as an initiation point for DNA synthesis. For example, a DNA polymerase adds nucleotides to the 3′ end of a primer to synthesize a new strand of DNA. Typically, the single stranded region of a primer is between about 10 to about 30 nucleotides in length.

For the methods provided herein, in embodiments, the DNA molecule is modified by contacting the DNA molecule and a PreQ1 analog or derivative as provided herein with TGT in a reaction mixture. In embodiments, the reaction mixture includes a suitable buffer system. In embodiments, the DNA molecule, TGT enzyme, and the PreQ1 analog or derivative are added to the reaction mixture simultaneously. In embodiments, the DNA molecule and the PreQ1 analog or derivative are added to the reaction mixture prior to adding the TGT enzyme. In embodiments, a DNA molecule may be modified in vivo (e.g. in a cell or in an organism). For example, a DNA molecule may be modified by expressing TGT within a cell (e.g. a mammalian cell). In embodiments, the PreQ1 analog or derivative and DNA molecule are exogenously added to the cell culture.

For the methods provided herein, in embodiments, Q is -L¹-L²-R³. L¹ and L² are independently a bond, —O—, —S—, —NH—, —C(O)NH₂, —NHC(O)—, —C(O)O—, —OC(O)—, —S(O)₂NH₂, —NHS(O)₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene, a peptide linker, or a cleavable linker.

R³ is a detectable moiety, a biomolecular moiety, a therapeutic moiety, hydrogen, halogen, —CX^{3.1}₃, —CHX^{3.1}₂, —CH₂X^{3.1}, —CN, —SOₙ₁R^{3A}, —SOᵥ₁NR^{3B}R^{3C}, —NHNR^{3B}R^{3C}, —ONR^{3B}R^{3C}, —NHC(O)NHNR^{3B}R^{3C}, —NHC(O)NR^{3B}R^{3C}, —N(O)ₘ₁, —NR^{3B}R^{3C}, —C(O)R^{3D}, —C(O)OR^{3D}, —C(O)NR^{3B}R^{3C}, —OR^{3A}, —NR^{3B}SO₂R^{3A}, —NR^{3B}C(O)R^{3D}, —NR^{3B}C(O)OR^{3D}, —NR^{3B}OR^{3D}, —OCX^{3.1}₃, —OCHX^{3.1} ₂ substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{3A}, R^{3B}, R^{3C} and R^{3D} are independently hydrogen, halogen, —CF₃, —CCl₃, —CBr₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3B} and R^{3C} substituents bonded to the same nitrogen atom may optionally be joined to form a substituted or unsubstituted heterocycloalkyl or substituted or unsubstituted heteroaryl.

X^{3.1} is independently-Cl, —Br, —I or —F.

In embodiments, L¹ is a
bond, —C(O)—, —C(O)O—, —OC(O)—, —O—, —S—, —NH—, —C(O)NH—,
—NHC(O)—, —NHC(O)O—, —OC(O)NH—, —NHC(O)NH—, —NHC(NH)NH—, —S(O)₂—, —NHS(O)₂—,
—S(O)₂NH—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), substituted or unsubstituted heteroarylene (e.g., 5 to 10 membered, 5 to 9 membered, or 5 to 6 membered), a peptide linker, or a cleavable linker.

In embodiments, L¹ is a bond. In embodiments, L¹ is —C(O)—. In embodiments, L¹ is —C(O)O—. In embodiments, L¹ is —OC(O)—. In embodiments, L¹ is —O—. In embodiments, L¹ is —S—. In embodiments, L¹ is —NH—. In embodiments, L¹ is —C(O)NH—. In embodiments, L¹ is —NHC(O)—. In embodiments, L¹ is —NHC(O)O—. In embodiments, L¹ is —OC(O)NH—. In embodiments, L¹ is —NHC(O)NH—. In embodiments, L¹ is —NHC(NH)NH—. In embodiments, L¹ is —S(O)₂—. In embodiments, L¹ is —NHS(O)₂—. In embodiments, L¹ is —S(O)₂NH—. In embodiments, L¹ is substituted or unsubstituted alkylene. In embodiments, L¹ is unsubstituted C₁-C₁₀ alkylene. In embodiments, L¹ is unsubstituted methylene. In embodiments, L¹ is unsubstituted ethylene. In embodiments, L¹ is unsubstituted propylene. In embodiments, L¹ is unsubstituted n-propylene. In embodiments, L¹ is unsubstituted butylene. In embodiments, L¹ is unsubstituted n-butylene. In embodiments, L¹ is substituted or unsubstituted heteroalkylene. In embodiments, L¹ is substituted or unsubstituted 2 to 16 membered heteroalkylene. In embodiments, L¹ is substituted or unsubstituted 2 to 10 membered heteroalkylene. In embodiments, L¹ is substituted (e.g., oxo-substituted) 2 to 16 membered heteroalkylene. In embodiments, L¹ is substituted (e.g., oxo-substituted) 2 to 10 membered heteroalkylene. In embodiments, L¹ is -(unsubstituted C₁-C₁₀ alkylene)-NHC(O)—. In embodiments, L¹ is —(CH₂)—NHC(O)—. In embodiments, L¹ is —(CH₂)₂—NHC(O)—. In embodiments, L¹ is —(CH₂)₃—NHC(O)—. In embodiments, L¹ is —(CH₂)₄—NHC(O)—. In embodiments, L¹ is —(CH₂)₅—NHC(O)—. In embodiments, L¹ is —(CH₂)₆—NHC(O)—. In embodiments, L¹ is —(CH₂)₇—NHC(O)—. In embodiments, L¹ is —(CH₂)₈—NHC(O)—. In embodiments, L¹ is —(CH₂)₉—NHC(O)—. In embodiments, L¹ is —(CH₂)₁₀—NHC(O)—. In embodiments, L¹ is -(unsubstituted C₁-C₁₀ alkylene)-NHC(O)O—. In embodiments, L¹ is
—(CH₂)—NHC(O)O—. In embodiments, L¹ is —(CH₂)₂—NHC(O)O—. In embodiments, L¹ is
—(CH₂)₃—NHC(O)O—. In embodiments, L¹ is —(CH₂)₄—NHC(O)O—. In embodiments, L¹ is
—(CH₂)₅—NHC(O)O—. In embodiments, L¹ is —(CH₂)₆—NHC(O)O—. In embodiments, L¹ is
—(CH₂)₇—NHC(O)O—. In embodiments, L¹ is —(CH₂)₈—NHC(O)O—. In embodiments, L¹ is
—(CH₂)₉—NHC(O)O—. In embodiments, L¹ is —(CH₂)₁₀—NHC(O)O—. In embodiments, L¹ is
-(unsubstituted C₁-C₁₀ alkylene)-NHC(O)O-(unsubstituted C₁-C₁₀ alkylene)-. In embodiments, L¹ is —(CH₂)—NHC(O)O—C(CH₃)—. In embodiments, L¹ is —(CH₂)₂—NHC(O)O—C(CH₃)—. In embodiments, L¹ is —(CH₂)₃—NHC(O)O—C(CH₃)—. In embodiments, L¹ is
—(CH₂)₄—NHC(O)O—C(CH₃)—. In embodiments, L¹ is —(CH₂)₅—NHC(O)O—C(CH₃)—. In embodiments, L¹ is —(CH₂)₆—NHC(O)O—C(CH₃)—. In embodiments, L¹ is
—(CH₂)₇—NHC(O)O—C(CH₃)—. In embodiments, L¹ is —(CH₂)₈—NHC(O)O—C(CH₃)—. In embodiments, L¹ is —(CH₂)₉—NHC(O)O—C(CH₃)—. In embodiments, L¹ is
—(CH₂)₁₀—NHC(O)O—C(CH₃)—.

In embodiments, L² is a
bond, —C(O)—, —C(O)O—, —OC(O)—, —O—, —S—, —NH—, —C(O)NH—,
—NHC(O)—, —NHC(O)O—, —OC(O)NH—, —NHC(O)NH—, —NHC(NH)NH—, —S(O)₂—, —NHS(O)₂—,
—S(O)₂NH—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), substituted or unsubstituted heteroarylene (e.g., 5 to 10 membered, 5 to 9 membered, or 5 to 6 membered), a peptide linker, or a cleavable linker.

In embodiments, L² is a bond. In embodiments, L² is —C(O)—. In embodiments, L² is —C(O)O—. In embodiments, L² is —OC(O)—. In embodiments, L² is —O—. In embodiments, L² is —S—. In embodiments, L² is —NH—. In embodiments, L² is —C(O)NH—. In embodiments, L² is —NHC(O)—. In embodiments, L² is —NHC(O)O—. In embodiments, L² is —OC(O)NH—. In embodiments, L² is —NHC(O)NH—. In embodiments, L² is —NHC(NH)NH—. In embodiments, L² is —S(O)₂—. In embodiments, L² is —NHS(O)₂—. In embodiments, L² is —S(O)₂NH—. In embodiments, L² is substituted or unsubstituted alkylene. In embodiments, L² is unsubstituted C₁-C₁₀ alkylene. In embodiments, L² is unsubstituted methylene. In embodiments, L² is unsubstituted ethylene. In embodiments, L² is unsubstituted propylene. In embodiments, L² is unsubstituted n-propylene. In embodiments, L² is unsubstituted butylene. In embodiments, L² is unsubstituted n-butylene. In embodiments, L² is substituted or unsubstituted heteroalkylene. In embodiments, L² is substituted or unsubstituted 2 to 16 membered heteroalkylene. In embodiments, L² is substituted or unsubstituted 2 to 10 membered heteroalkylene. In embodiments, L² is substituted (e.g., oxo-substituted) 2 to 16 membered heteroalkylene. In embodiments, L² is substituted (e.g., oxo-substituted) 2 to 10 membered heteroalkylene. In embodiments, L² is -(unsubstituted C₁-C₁₀ alkylene)-NHC(O)—. In embodiments, L² is —(CH₂)—NHC(O)—. In embodiments, L² is —(CH₂)₂—NHC(O)—. In embodiments, L² is —(CH₂)₃—NHC(O)—. In embodiments, L² is —(CH₂)₄—NHC(O)—. In embodiments, L² is —(CH₂)₅—NHC(O)—. In embodiments, L² is —(CH₂)₆—NHC(O)—. In embodiments, L² is —(CH₂)₇—NHC(O)—. In embodiments, L² is —(CH₂)₈—NHC(O)—. In embodiments, L² is —(CH₂)₉—NHC(O)—. In embodiments, L² is —(CH₂)₁₀—NHC(O)—. In embodiments, L² is -(unsubstituted C₁-C₁₀ alkylene)-NHC(O)O—. In embodiments, L² is
—(CH₂)—NHC(O)O—. In embodiments, L² is —(CH₂)₂—NHC(O)O—. In embodiments, L² is
—(CH₂)₃—NHC(O)O—. In embodiments, L² is —(CH₂)₄—NHC(O)O—. In embodiments, L² is
—(CH₂)₅—NHC(O)O—. In embodiments, L² is —(CH₂)₆—NHC(O)O—. In embodiments, L² is
—(CH₂)₇—NHC(O)O—. In embodiments, L² is —(CH₂)₈—NHC(O)O—. In embodiments, L² is
—(CH₂)₉—NHC(O)O—. In embodiments, L² is —(CH₂)₁₀—NHC(O)O—. In embodiments, L² is
-(unsubstituted C₁-C₁₀ alkylene)-NHC(O)O-(unsubstituted C₁-C₁₀ alkylene)-. In embodiments, L² is —(CH₂)—NHC(O)O—C(CH₃)—. In embodiments, L² is —(CH₂)₂—NHC(O)O—C(CH₃)—. In embodiments, L² is —(CH₂)₃—NHC(O)O—C(CH₃)—. In embodiments, L² is
—(CH₂)₄—NHC(O)O—C(CH₃)—. In embodiments, L² is —(CH₂)₅—NHC(O)O—C(CH₃)—. In embodiments, L² is —(CH₂)₆—NHC(O)O—C(CH₃)—. In embodiments, L² is
—(CH₂)₇—NHC(O)O—C(CH₃)—. In embodiments, L² is —(CH₂)₈—NHC(O)O—C(CH₃)—. In embodiments, L² is —(CH₂)₉—NHC(O)O—C(CH₃)—. In embodiments, L² is
—(CH₂)₁₀—NHC(O)O—C(CH₃)—.

For the methods provided herein, in embodiments, L¹ or L² includes a photo-cleavable site. In embodiments, L¹ includes a photo-cleavable site. In embodiments, L² includes a photo-cleavable site.

For the methods provided herein, in embodiments, L¹ or L² comprises a peptide linker. In embodiments, L¹ includes a peptide linker. In embodiments, L² includes a peptide linker. In embodiments, the peptide linker is about 3 to about 60 amino acid residues in length. In embodiments, peptide linker comprises a protease cleavable site. In embodiments, the protease cleavable site is a matrix metalloprotease (MMP) cleavage site, a metalloprotease domain-containing (ADAM) metalloprotease cleavage site, a lysosomal cathepsin cleavage site (ALAL), or a legumain endopeptidase cleavage site (TANL). In embodiments, the protease cleavable site is a MMP cleavage site. In embodiments, the protease cleavable site is an ADAM metalloprotease cleavage site. In embodiments, the protease cleavable site is an ALAL cleavage site. In embodiments, the protease cleavable site is a TANL cleavage site.

For the methods provided herein, in embodiments, L¹ or L² may include a pH sensitive site, thereby allowing release of a functional moiety (e.g. a therapeutic moiety) in an acidic environment (e.g. in a tumor). In embodiments, L¹ or L² includes a pH sensitive cleavable site. In embodiments, L¹ includes a pH sensitive cleavable site. In embodiments, L² includes a pH sensitive cleavable site. In embodiments, the pH cleavable site is cleaved at pH 6.5 or lower.

Methods provided herein are contemplated to be useful for detecting, quantifying or isolating a target oligonucleotide. For example, incorporation of a biotin modified preQ1 analog or derivative into a DNA molecule generates a DNA compound useful for isolating a target oligonuclotide. In another example, incorporating a detectable moiety modified preQ1 analog or derivative into a DNA molecule generates a DNA compound useful for detecting or quantifying a target oligonucleotide. Methods provided herein are further contemplated to be useful for modifying DNA molecules for DNA labeling and as visualization agents in live cells. For example, DNA molecules may be modified with bioorthogonal covalent labeling partners such as alkynes, azides, cyclopropenes, trancyclooctenes, and tetrazines by exchange of a guanine nucleobase with a PreQ1 analog or derivative as provided herein. In another example, fluorescent moieties attached to PreQ1 analogs may be used to isolate or identify nucleic acids of interest, for example by FISH. Thus, in embodiments, R³ is a detectable moiety. In embodiments, the detectable moiety is a fluorescent moiety. In embodiments, the fluorescent moiety is Thiazole Orange (TO) or a derivative thereof. Thus, in embodiments, a fluorescent moiety may be linked to a preQ1 analog via an alkyl linker or a PEG linker. In embodiments, substituting a guanine nucleobase within a DNA molecule with the preQ1 analog allows for intra-molecular interactions of the fluorescent moiety with the hairpin structure within the DNA molecule. Thus, in embodiments, the fluorescent moiety is Thiazole Orange (TO).

As described throughout the specification, including in the example and figures, the methods provided herein are useful for substituting substituting a guanine nucleobase within a DNA molecule with a PreQ1 analog. In an aspect is provided a method of substituting a guanine nucleobase within a DNA molecule with a PreQ1 analog, the method comprising contacting the DNA molecule and PreQ1 analog with a tRNA-guanine transglycosylase (TGT) enzyme,

wherein the guanine nucleobase is within a loop portion of a hairpin in the DNA molecule, and
wherein the PreQ1 analog has the formula:

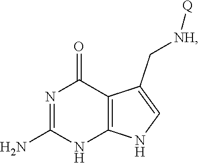

wherein Q is not hydrogen.

In another aspect is provided a method of substituting a guanine nucleobase within a DNA molecule with a PreQ1 derivative, the method including contacting the DNA molecule and PreQ1 derivative with a tRNA-guanine transglycosylase (TGT) enzyme, wherein the guanine nucleobase is within a loop portion of a hairpin in the DNA molecule, and wherein the PreQ1 derivative has the formula:

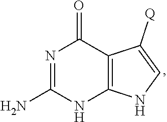

wherein Q is not hydrogen.

In embodiments, the guanine nucleobase is in a YYGYYYY (SEQ ID NO: 103) sequence within the loop portion of the hairpin in the DNA molecule. In embodiments, the sequence within the loop portion of the hairpin is YTGTYYY (SEQ ID NO:104), YTGTCCY (SEQ ID NO:105), YTGTYCC (SEQ ID NO:106), YUGUYYY (SEQ ID NO:107), YUGTYYY (SEQ ID NO:108), or YTGUYYY (SEQ ID NO:109). In embodiments, the sequence within the loop portion of the hairpin is YTGTYYY (SEQ ID NO:104). In embodiments, the sequence within the loop portion of the hairpin is YTGTCCY (SEQ ID NO:105). In embodiments, the sequence within the loop portion of the hairpin is YTGTYCC (SEQ ID NO:106). In embodiments, the sequence within the loop portion of the hairpin is YUGUYYY (SEQ ID NO:107). In embodiments, the sequence within the loop portion of the hairpin is YUGTYYY (SEQ ID NO:108), or YTGUYYY (SEQ ID NO:109). In embodiments, the sequence within the loop portion of the hairpin is YTGUYYY (SEQ ID NO:109). In embodiments, the sequence within the loop portion of the hairpin is YTGTCCY (SEQ ID NO:105) or YTGTYCC (SEQ ID NO:106).

In embodiments, the DNA molecule is between about 15 nucleotides to about 30,000 nucleotides in length. In embodiments, the DNA molecule is between about 15 nucleotides to about 10,000 nucleotides in length. In embodiments, the DNA molecule is between about 15 nucleotides to about 150 nucleotides in length. In embodiments, the DNA molecule is between about 5 nucleotides to about 50 nucleotides in length.

In embodiments, the DNA molecule is a gene, a cDNA, a non-coding DNA, an extracellular DNA (eDNA), an antisense oligonucleotide, a barcode, a probe, or a primer. In embodiments, the DNA molecule is a gene. In embodiments, the DNA molecule is a cDNA. In embodiments, the DNA molecule is a a non-coding DNA. In embodiments, the DNA molecule is an extracellular DNA (eDNA). In embodiments, the DNA molecule is an antisense oligonucleotide. In embodiments, the DNA molecule is a barcode. In embodiments, the DNA molecule is a a probe. In embodiments, the DNA molecule is a primer.

In embodiments, the DNA molecule, TGT enzyme, and the PreQ1 analog are added to the reaction mixture simultaneously. In embodiments, the DNA molecule and the PreQ1 analog are added to the reaction mixture prior to adding the TGT enzyme.

For the methods provided herein, in embodiments, Q is -L¹-L²-R³. L¹ and L² are independently a bond, —O—, —S—, —NH—, —C(O)NH₂, —NHC(O)—, —C(O)O—, —OC(O)—, —S(O)₂NH₂, —NHS(O)₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene, a peptide linker, or a cleavable linker.

R³ is a detectable moiety, a biomolecular moiety, a therapeutic moiety, hydrogen, halogen, —CX^{3.1}₃, —CHX^{3.1}₂, —CH₂X^{3.1}, —CN, —SOₙ₁R^{3A}, —SOᵥ₁NR^{3B}R^{3C}, —NHNR^{3B}R^{3C}, —ONR^{3B}R^{3C}, —NHC(O)NHNR^{3B}R^{3C}, —NHC(O)NR^{3B}R^{3C}. —N(O)ₘ₁, —NR^{3B}R^{3C}, —C(O)R^{3D}, —C(O)OR^{3D}, —C(O)NR^{3B}R^{3C}, —OR^{3A}, —NR^{3B}SO₂R^{3A}, —NR^{3B}C(O)R^{3D}, —NR^{3B}C(O)OR^{3D}, —NR^{3B}OR^{3D}, —OCX^{3.1}₃, —OCHX^{3.1} ₂ substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{3A}, R^{3B}, R^{3C} and R^{3D} are independently hydrogen, halogen, —CF₃, —CCl₃, —CBr₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3B} and R^{3C} substituents bonded to the same nitrogen atom may optionally be joined to form a substituted or unsubstituted heterocycloalkyl or substituted or unsubstituted heteroaryl.

X^{3.1} is independently-Cl, —Br, —I or —F.

In embodiments, L¹ is a
bond, —C(O)—, —C(O)O—, —OC(O)—, —O—, —S—, —NH—, —C(O)NH—,
—NHC(O)—, —NHC(O)O—, —OC(O)NH—, —NHC(O)NH—, —NHC(NH)NH—, —S(O)₂—, —NHS(O)₂—,
—S(O)₂NH—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), substituted or unsubstituted heteroarylene (e.g., 5 to 10 membered, 5 to 9 membered, or 5 to 6 membered), a peptide linker, or a cleavable linker.

In embodiments, L¹ is a bond. In embodiments, L¹ is —C(O)—. In embodiments, L¹ is —C(O)O—. In embodiments, L¹ is —OC(O)—. In embodiments, L¹ is —O—. In embodiments, L¹ is —S—. In embodiments, L¹ is —NH—. In embodiments, L¹ is —C(O)NH—. In embodiments, L¹ is —NHC(O)—. In embodiments, L¹ is —NHC(O)O—. In embodiments, L¹ is —OC(O)NH—. In embodiments, L¹ is —NHC(O)NH—. In embodiments, L¹ is —NHC(NH)NH—. In embodiments, L¹ is —S(O)₂—. In embodiments, L¹ is —NHS(O)₂—. In embodiments, L¹ is —S(O)₂NH—. In embodiments, L¹ is substituted or unsubstituted alkylene. In embodiments, L¹ is unsubstituted C₁-C₁₀ alkylene. In embodiments, L¹ is unsubstituted methylene. In embodiments, L¹ is unsubstituted ethylene. In embodiments, L¹ is unsubstituted propylene. In embodiments, L¹ is unsubstituted n-propylene. In embodiments, L¹ is unsubstituted butylene. In embodiments, L¹ is unsubstituted n-butylene. In embodiments, L¹ is substituted or unsubstituted heteroalkylene. In embodiments, L¹ is substituted or unsubstituted 2 to 16 membered heteroalkylene. In embodiments, L¹ is substituted or unsubstituted 2 to 10 membered heteroalkylene. In embodiments, L¹ is substituted (e.g., oxo-substituted) 2 to 16 membered heteroalkylene. In embodiments, L¹ is substituted (e.g., oxo-substituted) 2 to 10 membered heteroalkylene. In embodiments, L¹ is -(unsubstituted C₁-C₁₀ alkylene)-NHC(O)—. In embodiments, L¹ is —(CH₂)—NHC(O)—. In embodiments, L¹ is —(CH₂)₂—NHC(O)—. In embodiments, L¹ is —(CH₂)₃—NHC(O)—. In embodiments, L¹ is —(CH₂)₄—NHC(O)—. In embodiments, L¹ is —(CH₂)₅—NHC(O)—. In embodiments, L¹ is —(CH₂)₆—NHC(O)—. In embodiments, L¹ is —(CH₂)₇—NHC(O)—. In embodiments, L¹ is —(CH₂)₈—NHC(O)—. In embodiments, L¹ is —(CH₂)₉—NHC(O)—. In embodiments, L¹ is —(CH₂)₁₀—NHC(O)—. In embodiments, L¹ is -(unsubstituted C₁-C₁₀ alkylene)-NHC(O)O—. In embodiments, L¹ is
—(CH₂)—NHC(O)O—. In embodiments, L¹ is —(CH₂)₂—NHC(O)O—. In embodiments, L¹ is
—(CH₂)₃—NHC(O)O—. In embodiments, L¹ is —(CH₂)₄—NHC(O)O—. In embodiments, L¹ is
—(CH₂)₅—NHC(O)O—. In embodiments, L¹ is —(CH₂)₆—NHC(O)O—. In embodiments, L¹ is
—(CH₂)₇—NHC(O)O—. In embodiments, L¹ is —(CH₂)₈—NHC(O)O—. In embodiments, L¹ is
—(CH₂)₉—NHC(O)O—. In embodiments, L¹ is —(CH₂)₁₀—NHC(O)O—. In embodiments, L¹ is
-(unsubstituted C₁-C₁₀ alkylene)-NHC(O)O-(unsubstituted C₁-C₁₀ alkylene)-. In embodiments, L¹ is —(CH₂)—NHC(O)O—C(CH₃)—. In embodiments, L¹ is —(CH₂)₂—NHC(O)O—C(CH₃)—. In embodiments, L¹ is —(CH₂)₃—NHC(O)O—C(CH₃)—. In embodiments, L¹ is
—(CH₂)₄—NHC(O)O—C(CH₃)—. In embodiments, L¹ is —(CH₂)₅—NHC(O)O—C(CH₃)—. In embodiments, L¹ is —(CH₂)₆—NHC(O)O—C(CH₃)—. In embodiments, L¹ is
—(CH₂)₇—NHC(O)O—C(CH₃)—. In embodiments, L¹ is —(CH₂)₉—NHC(O)O—C(CH₃)—. In embodiments, L¹ is —(CH₂)₉—NHC(O)O—C(CH₃)—. In embodiments, L¹ is
—(CH₂)₁₀—NHC(O)O—C(CH₃)—.

In embodiments, L² is a
bond, —C(O)—, —C(O)O—, —OC(O)—, —O—, —S—, —NH—, —C(O)NH—,
—NHC(O)—, —NHC(O)O—, —OC(O)NH—, —NHC(O)NH—, —NHC(NH)NH—, —S(O)₂—, —NHS(O)₂—,
—S(O)₂NH—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), substituted or unsubstituted heteroarylene (e.g., 5 to 10 membered, 5 to 9 membered, or 5 to 6 membered), a peptide linker, or a cleavable linker.

In embodiments, L² is a bond. In embodiments, L² is —C(O)—. In embodiments, L² is —C(O)O—. In embodiments, L² is —OC(O)—. In embodiments, L² is —O—. In embodiments, L² is —S—. In embodiments, L² is —NH—. In embodiments, L² is —C(O)NH—. In embodiments, L² is —NHC(O)—. In embodiments, L² is —NHC(O)O—. In embodiments, L² is —OC(O)NH—. In embodiments, L² is —NHC(O)NH—. In embodiments, L² is —NHC(NH)NH—. In embodiments, L² is —S(O)₂—. In embodiments, L² is —NHS(O)₂—. In embodiments, L² is —S(O)₂NH—. In embodiments, L² is substituted or unsubstituted alkylene. In embodiments, L² is unsubstituted C₁-C₁₀ alkylene. In embodiments, L² is unsubstituted methylene. In embodiments, L² is unsubstituted ethylene. In embodiments, L² is unsubstituted propylene. In embodiments, L² is unsubstituted n-propylene. In embodiments, L² is unsubstituted butylene. In embodiments, L² is unsubstituted n-butylene. In embodiments, L² is substituted or unsubstituted heteroalkylene. In embodiments, L² is substituted or unsubstituted 2 to 16 membered heteroalkylene. In embodiments, L² is substituted or unsubstituted 2 to 10 membered heteroalkylene. In embodiments, L² is substituted (e.g., oxo-substituted) 2 to 16 membered heteroalkylene. In embodiments, L² is substituted (e.g., oxo-substituted) 2 to 10 membered heteroalkylene. In embodiments, L² is -(unsubstituted C₁-C₁₀ alkylene)-NHC(O)—. In embodiments, L² is —(CH₂)—NHC(O)—. In embodiments, L² is —(CH₂)₂—NHC(O)—. In embodiments, L² is —(CH₂)₃—NHC(O)—. In embodiments, L² is —(CH₂)₄—NHC(O)—. In embodiments, L² is —(CH₂)₅—NHC(O)—. In embodiments, L² is —(CH₂)₆—NHC(O)—. In embodiments, L² is —(CH₂)₇—NHC(O)—. In embodiments, L² is —(CH₂)₈—NHC(O)—. In embodiments, L² is —(CH₂)₉—NHC(O)—. In embodiments, L² is —(CH₂)₁₀—NHC(O)—. In embodiments, L² is -(unsubstituted C₁-C₁₀ alkylene)-NHC(O)O—. In embodiments, L² is
—(CH₂)—NHC(O)O—. In embodiments, L² is —(CH₂)₂—NHC(O)O—. In embodiments, L² is
—(CH₂)₃—NHC(O)O—. In embodiments, L² is —(CH₂)₄—NHC(O)O—. In embodiments, L² is
—(CH₂)₅—NHC(O)O—. In embodiments, L² is —(CH₂)₆—NHC(O)O—. In embodiments, L² is
—(CH₂)₇—NHC(O)O—. In embodiments, L² is —(CH₂)₈—NHC(O)O—. In embodiments, L² is
—(CH₂)₉—NHC(O)O—. In embodiments, L² is —(CH₂)₁₀—NHC(O)O—. In embodiments, L² is
-(unsubstituted C₁-C₁₀ alkylene)-NHC(O)O-(unsubstituted C₁-C₁₀ alkylene)-. In embodiments, L² is —(CH₂)—NHC(O)O—C(CH₃)—. In embodiments, L² is —(CH₂)₂—NHC(O)O—C(CH₃)—. In embodiments, L² is —(CH₂)₃—NHC(O)O—C(CH₃)—. In embodiments, L² is
—(CH₂)₄—NHC(O)O—C(CH₃)—. In embodiments, L² is —(CH₂)₅—NHC(O)O—C(CH₃)—. In embodiments, L² is —(CH₂)₆—NHC(O)O—C(CH₃)—. In embodiments, L² is
—(CH₂)₇—NHC(O)O—C(CH₃)—. In embodiments, L² is —(CH₂)₅—NHC(O)O—C(CH₃)—. In embodiments, L² is —(CH₂)₉—NHC(O)O—C(CH₃)—. In embodiments, L² is
—(CH₂)₁₀—NHC(O)O—C(CH₃)—.

For the methods provided herein, in embodiments, L¹ or L² includes a photo-cleavable site. In embodiments, L¹ includes a photo-cleavable site. In embodiments, L² includes a photo-cleavable site.

In embodiments, L¹ or L² includes a peptide linker. In embodiments, L¹ includes a peptide linker. In embodiments, L² includes a peptide linker. In embodiments, the peptide linker is about 3 to about 60 amino acid residues in length. In embodiments, peptide linker comprises a protease cleavable site. In embodiments, the protease cleavable site is a matrix metalloprotease (MMP) cleavage site, a metalloprotease domain-containing (ADAM) metalloprotease cleavage site, a lysosomal cathepsin cleavage site (ALAL), or a legumain endopeptidase cleavage site (TANL). In embodiments, the protease cleavable site is a MMP cleavage site. In embodiments, the protease cleavable site is an ADAM metalloprotease cleavage site. In embodiments, the protease cleavable site is an ALAL cleavage site. In embodiments, the protease cleavable site is a TANL cleavage site.

In embodiments, L¹ or L² includes a pH sensitive cleavable site. In embodiments, L¹ includes a pH sensitive cleavable site. In embodiments, L² includes a pH sensitive cleavable site. In embodiments, the pH cleavable site is cleaved at pH 6.5 or lower.

In embodiments, R³ is a detectable moiety. In embodiments, the detectable moiety is a fluorescent moiety.

### Cells

The compositions provided herein, including the preQ1 analog, the preQ1 derivative, and the DNA compound (e.g. the DNA compound of formula I-VIII), may be introduced into cells. For example, the DNA compound provided herein may be generated within a cell or delivered into a cell, allowing detection of the cell or identification of a target oligonucleotide within the cell. Thus, in an aspect a cell including DNA compound provided herein including embodiments thereof is provided. In another aspect, a cell including a PreQ1 analog as provided herein including embodiments thereof is provided. In embodiments, the cell includes a tRNA-guanine transglycosylase (TGT) enzyme.

### P Embodiments

P embodiment 1: A method of modifying DNA, the method comprising: contacting a DNA molecule with a transglycosylase and a nucleoside derivative under conditions suitable to exchange at least one guanine nucleobase of the DNA for the nucleoside derivative, said DNA molecule comprising a hairpin comprising a loop sequence of 5′ YYGYYYY 3′ (SEQ ID NO: 103).

P embodiment 2: The method of claim 1, wherein the loop sequence is 5′ YTGTYYY 3′ (SEQ ID NO:104), 5′ YTGTCCY 3′ (SEQ ID NO:105), 5′ YTGTYCC 3′ (SEQ ID NO:106), 5′ YUGUYYY 3′ (SEQ ID NO:107), 5′ YUGTYYY 3′ (SEQ ID NO:108) or 5′ YTGUYYY 3′ (SEQ ID NO:109).

P embodiment 3: The method of embodiment 1 or 2, wherein the transglycosylase is a bacterial tRNA guanine glycosylase.

P embodiment 4: A method of substituting a guanine with a PreQ1 analog within a DNA molecule, comprising i) contacting a PreQ1 analog with an DNA molecule in the presence of a transglycosylase; and ii) allowing the transglycosylase to substitute a guanine moiety from a guanine within the DNA sequence with the PreQ1 analog thereby forming a modified DNA molecule.

P embodiment 5: The method of embodiment 4, wherein the guanine is within a hairpin loop in the DNA molecule.

P embodiment 6: The method of embodiment 4 or 5, wherein the guanine is in a 5′ YYGYYYY 3′ sequence (SEQ ID NO: 103).

P embodiment 7: The method of embodiment 6, wherein the guanine is in a 5′ YTGTCCY 3′ (SEQ ID NO:105), 5′ YTGTYCC 3′ (SEQ ID NO:106), 5′ YUGUYYY 3′ (SEQ ID NO:107), 5′ YUGTYYY 3′ (SEQ ID NO:108) or 5′ YTGUYYY 3′ (SEQ ID NO:109)sequence.

P embodiment 8: A compound having the formula:

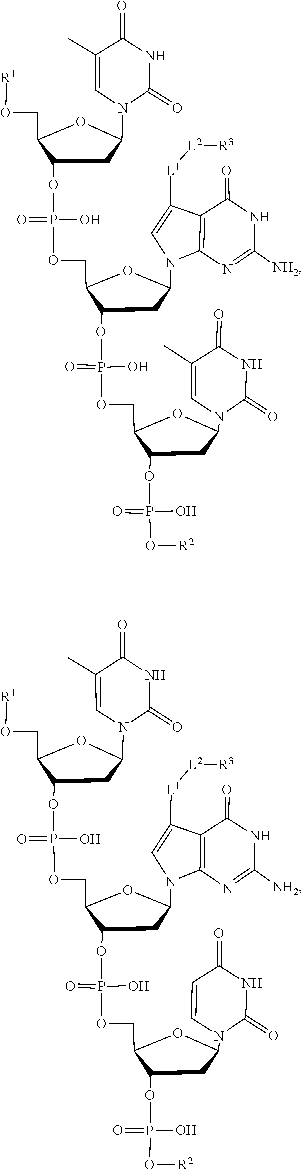

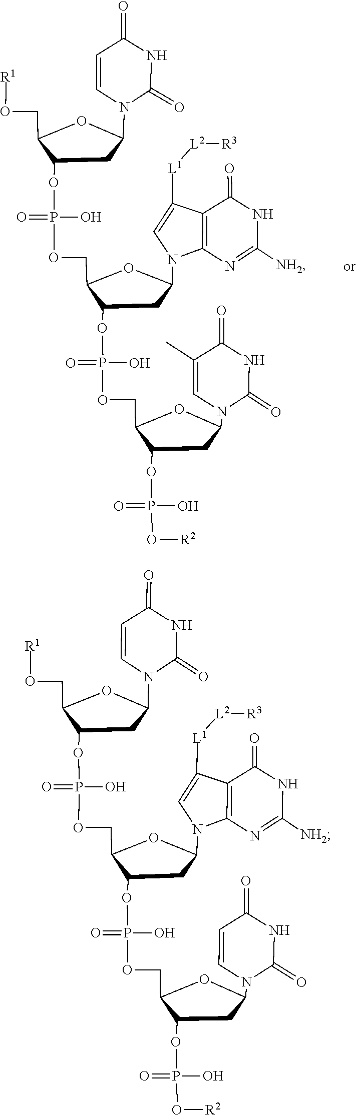

R¹ and R² are independently hydrogen or a deoxynucleotide;
L¹ and L² are independently a bond, —O—, —S—, —NH—, —C(O)NH₂, —NHC(O)—, —C(O)O—, —OC(O)—, —S(O)₂NH₂, —NHS(O)₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R³ is a detectable moiety, a biomolecular moiety, a therapeutic moiety, hydrogen, halogen, —CX^{3.1}₃, —CHX^{3.1}₂, —CH₂X^{3.1}, —CN, —SOₙ₁R^{3A}, —SOᵥ₁NR^{3B}R^{3C}, —NHNR^{3B}R^{3C}, —ONR^{3B}R^{3C}, —NHC(O)NHNR^{3B}R^{3C}, —NHC(O)NR^{3B}R^{3C}, —N(O)ₘ₁, —NR^{3B}R^{3C}, —C(O)R^{3D}, —C(O)OR^{3D}, —C(O)NR^{3B}R^{3C}, —OR^{3A}, —NR^{3B}SO₂R^{3A}, —NR^{3B}C(O)R^{3D}, —NR^{3B}C(O)OR^{3D}, —NR^{3B}OR^{3D}, —OCX³, —OCHX^{3.1}₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3A}, R^{3B}, R^{3C} and R^{3D} are independently hydrogen, halogen, —CF₃, —CCl₃, —CBr₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —S04H, —SO₂NH₂, —NHNH₂, —ONH₂, —NHC(O)NHNH₂, —NHC(O)NH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3B} and R^{3C} substituents bonded to the same nitrogen atom may optionally be joined to form a substituted or unsubstituted heterocycloalkyl or substituted or unsubstituted heteroaryl; and X^{3.1} is independently-Cl, —Br, —I or —F.

P embodiment 9: The compound of embodiment Error! Reference source not found., having the formula:

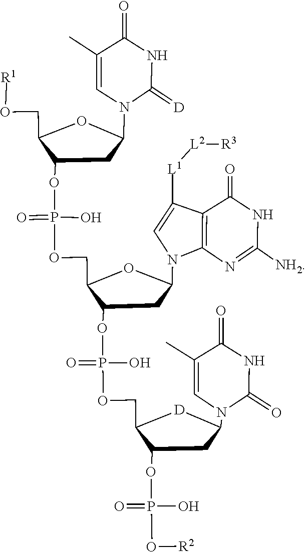

P embodiment 10: The compound of embodiment Error! Reference source

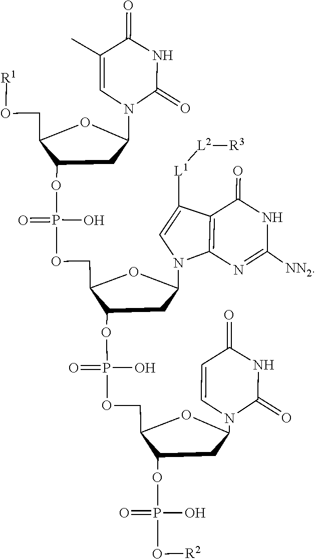

P embodiment 11: The compound of embodiment Error! Reference source not found., having the formula:

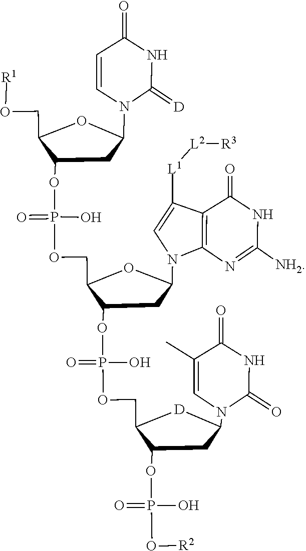

P embodiment 12: The compound of embodiment Error! Reference source not found., having the formula:

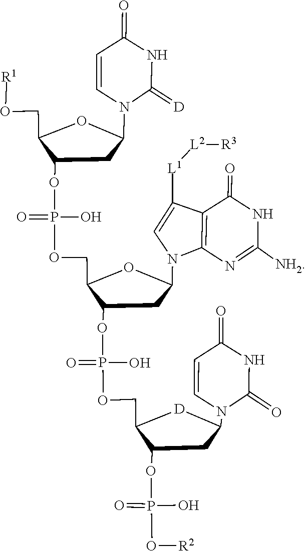

P embodiment 13: The compound of one of embodiments 8-12, wherein R¹ is hydrogen.

P embodiment 14: The compound of one of embodiments 8-12, wherein R¹ is a deoxynucleotide.

P embodiment 15: The compound of embodiment 14, wherein R¹ is a deoxyadenosine, deoxyguanosine, thymidine, deoxyuridine, or deoxycytidine.

P embodiment 16: The compound of one of embodiments 8-15, wherein R² is hydrogen.

P embodiment 17: The compound of one of embodiments 8-15, wherein R² is a deoxynucleotide.

P embodiment 18: The compound of embodiment 17, wherein R² is a deoxyadenosine, deoxyguanosine, thymidine, deoxyuridine, or deoxycytidine.

P embodiment 19: The compound of one of embodiments 8-18, wherein L¹ is a bond or unsubstituted 2 to 12 membered heteroalkylene.

P embodiment 20: The compound of one of embodiments 8-19, wherein L² is a bond or unsubstituted 2 to 12 membered heteroalkylene.

P embodiment 21: The compound of one of embodiments 8-20, wherein R³ is a detectable moiety, a biomolecular moiety, a therapeutic moiety, hydrogen, halogen, —CF3, —CCl₃, —CBr₃, —
CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —
OCHI₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

P embodiment 22: The compound of one of embodiments 8-20, wherein R³ is a detectable moiety.

### EMBODIMENTS

Embodiment 1: A method of modifying a DNA molecule, the method comprising contacting the DNA molecule with a tRNA-guanine transglycosylase (TGT) enzyme and a PreQ1 analog,

wherein the DNA molecule comprises a guanine nucleobase within a loop portion of a hairpin in the DNA molecule, and
wherein the PreQ1 analog has the formula:

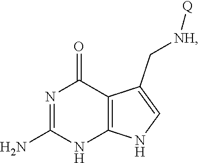

wherein Q is not hydrogen

Embodiment 2: The method of embodiment 1, wherein the guanine nucleobase is in a YYGYYYY (SEQ ID NO: 103) sequence within the loop portion of the hairpin in the DNA molecule.

Embodiment 3: The method of embodiment 2, wherein the sequence within the loop portion of the hairpin is YTGTYYY (SEQ ID NO:104), YTGTCCY (SEQ ID NO:105), YTGTYCC (SEQ ID NO:106), YUGUYYY (SEQ ID NO:107), YUGTYYY (SEQ ID NO:108), or YTGUYYY (SEQ ID NO:109).

Embodiment 4: The method of embodiment 3, wherein the sequence within the loop portion of the hairpin is YTGTCCY (SEQ ID NO:105) or YTGTYCC (SEQ ID NO:106).

Embodiment 5: The method of any one of embodiments 1-4, wherein the DNA molecule is between about 15 nucleotides to about 30,000 nucleotides in length.

Embodiment 6: The method of any one of embodiments 1-5, wherein the DNA molecule is between about 15 nucleotides to about 150 nucleotides in length.

Embodiment 7: The method of any one of embodiments 1-6, wherein the DNA molecule is a gene, a cDNA, a non-coding DNA, an extracellular DNA (eDNA), an antisense oligonucleotide, a barcode, a probe, or a primer.

Embodiment 8: The method of any one of embodiments 1-7, wherein the DNA molecule, TGT enzyme, and the PreQ1 analog are added to the reaction mixture simultaneously.

Embodiment 9: The method of any one of embodiments 1-8, wherein the DNA molecule and the PreQ1 analog are added to the reaction mixture prior to adding the TGT enzyme.

Embodiment 10: The method of any one of embodiments 1-9, wherein Q is -L ¹-L²-R³;
L¹ and L² are independently a bond, —O—, —S—, —NH—, —C(O)NH₂, —NHC(O)—, —C(O)O—, —OC(O)—. —S(O)₂NH₂, —NHS(O)₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene, a peptide linker, or a cleavable linker;
R³ is a detectable moiety, a biomolecular moiety, a therapeutic moiety, hydrogen, halogen, —CX^{3.1}, —CHX^{3.1}₂, —CH₂X^{3.1}, —CN, —SOₙ₁R^{3A}, —SOᵥ₁NR^{3B}R^{3C}, —NHNR^{3B}R^{3C}, —ONR^{3B}R^{3C}, —NHC(O)NHNR^{3B}R^{3C}, —NHC(O)NR^{3B}R^{3C}, —N(O)ₘ₁, —NR^{3B}R^{3C}, —C(O)R^{3D}, —C(O)OR^{3D}, —C(O)NR^{3B}R^{3C}, —OR^{3A}, —NR^{3B}SO₂R^{3A}, —NR^{3B}C(O)R^{3D}, —NR^{3B}C(O)OR^{3D}, —NR^{3B}OR^{3D}, —OCX^{3.1}₃, —OCHX^{3.1}₂ substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3A}, R^{3B}, R^{3C} and R^{3D} are independently hydrogen, halogen, —CF₃, —CCl₃, —CBr₃, —
CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —
OCHI₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3B} and R^{3C} substituents bonded to the same nitrogen atom may optionally be joined to form a substituted or unsubstituted heterocycloalkyl or substituted or unsubstituted heteroaryl; and
X^{3.1} is independently-Cl, —Br, —I or —F.

Embodiment 11: The method of embodiment 10, wherein L¹ or L² comprises a photo-cleavable site.

Embodiment 12: The method of embodiment 10, wherein L¹ or L² comprises a peptide linker.

Embodiment 13: The method of any embodiment 12, wherein said peptide linker comprises a protease cleavable site.

Embodiment 14: The method of embodiment 13, wherein said protease cleavable site is a matrix metalloprotease (MMP) cleavage site, a metalloprotease domain-containing (ADAM) metalloprotease cleavage site, a lysosomal cathepsin cleavage site (ALAL), or a legumain endopeptidase cleavage site (TANL).

Embodiment 15: The method of embodiment 10, wherein L¹ comprises a pH sensitive cleavable site.

Embodiment 16: The method of any one of embodiments 10-15, wherein R³ is a detectable moiety.

Embodiment 17: The method of embodiment 16, wherein the detectable moiety is a fluorescent moiety.

Embodiment 18: A method of substituting a guanine nucleobase within a DNA molecule with a PreQ1 analog, the method comprising contacting the DNA molecule and PreQ1 analog with a tRNA-guanine transglycosylase (TGT) enzyme, wherein the guanine nucleobase is within a loop portion of a hairpin in the DNA molecule, and

wherein the PreQ1 analog has the formula:

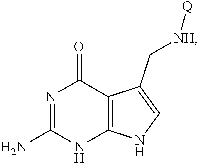

wherein Q is not hydrogen.

Embodiment 19: The method of embodiment 18, wherein the guanine nucleobase is in a YYGYYYY (SEQ ID NO: 103) sequence within the loop portion of the hairpin in the DNA molecule.

Embodiment 20: The method of embodiment 19, wherein the sequence within the loop portion of the hairpin is YTGTCCY (SEQ ID NO:105), YTGTYCC (SEQ ID NO:106), YUGUYYY (SEQ ID NO:107), YUGTYYY (SEQ ID NO:108) or YTGUYYY (SEQ ID NO: 109).

Embodiment 21: The method of embodiment 20, wherein the sequence within the loop portion of the hairpin is YTGTCCY (SEQ ID NO:105) or YTGTYCC (SEQ ID NO:106).

Embodiment 22: The method of any of embodiments 18-21, wherein the DNA molecule is between about 15 nucleotides to about 30,000 nucleotides in length.

Embodiment 23: The method of any of embodiments 18-22, wherein the DNA molecule is between about 15 nucleotides to about 150 nucleotides in length.

Embodiment 24: The method of any one of embodiments 18-23, wherein the DNA molecule is a gene, a cDNA, a non-coding DNA, an extracellular DNA (eDNA), an antisense oligonucleotide, a barcode, a probe, or a primer.

Embodiment 25: The method of any one of embodiments 18-24, wherein the DNA molecule, TGT enzyme, and the PreQ1 analog are added to the reaction mixture simultaneously.

Embodiment 26: The method of any one of embodiments 18-24, wherein the DNA molecule and the PreQ1 analog are added to the reaction mixture prior to adding the TGT enzyme.

Embodiment 27: The method of any one of embodiments 18-26, wherein Q is -L¹-L²-R³;
L¹ and L² are independently a bond, —O—, —S—, —NH—, —C(O)NH₂, —NHC(O)—, —C(O)O—, —OC(O)—, —S(O)₂NH₂, —NHS(O)₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene, a peptide linker, or a cleavable linker;
R³ is a detectable moiety, a biomolecular moiety, a therapeutic moiety, hydrogen, halogen, —CX^{3.1}₃, —CHX^{3.1}₂, —CH₂X^{3.1}, —CN, —SOₙ₁R^{3A}, —SOᵥ₁NR^{3B}R^{3C}, —NHNR^{3B}R^{3C}, —ONR^{3B}R^{3C}, —NHC(O)NHNR^{3B}R^{3C}, —NHC(O)NR^{3B}R^{3C}, —N(O)ₘ₁, —NR^{3B}R^{3C}, —C(O)R^{3D}, —C(O)OR^{3D}, —C(O)NR^{3B}R^{3C}, —OR^{3A}, —NR^{3B}SO₂R^{3A}, —NR^{3B}C(O)R^{3D}, —NR^{3B}C(O)OR^{3D}, —NR^{3B}OR^{3D}, —OCX^{3.1}₃, —OCHX^{3.1}₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3A}, R^{3B}, R^{3C} and R^{3D} are independently hydrogen, halogen, —CF₃, —CCl₃, —CBr₃, —
CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —
OCHI₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3B} and R^{3C} substituents bonded to the same nitrogen atom may optionally be joined to form a substituted or unsubstituted heterocycloalkyl or substituted or unsubstituted heteroaryl; and
X^{3.1} is independently-Cl, —Br, —I or —F.

Embodiment 28: The method of embodiment 27, wherein L¹ or L² comprises a photo-cleavable site.

Embodiment 29: The method of embodiment 27, wherein L¹ or L² comprises a peptide linker.

Embodiment 30: The method of embodiment 29, wherein said peptide linker comprises a protease cleavable site.

Embodiment 31: The method of embodiment 30, wherein said protease cleavable site is a matrix metalloprotease (MMP) cleavage site, a metalloprotease domain-containing (ADAM) metalloprotease cleavage site, a lysosomal cathepsin cleavage site (ALAL), or a legumain endopeptidase cleavage site (TANL).

Embodiment 32: The method of embodiment 27, wherein L¹ comprises a pH sensitive cleavable site.

Embodiment 33: The method of any one of embodiments 27-32, wherein R³ is a detectable moiety.

Embodiment 34: The method of embodiment 33, wherein the detectable moiety is a fluorescent moiety.

Embodiment 35: A DNA compound having the formula:

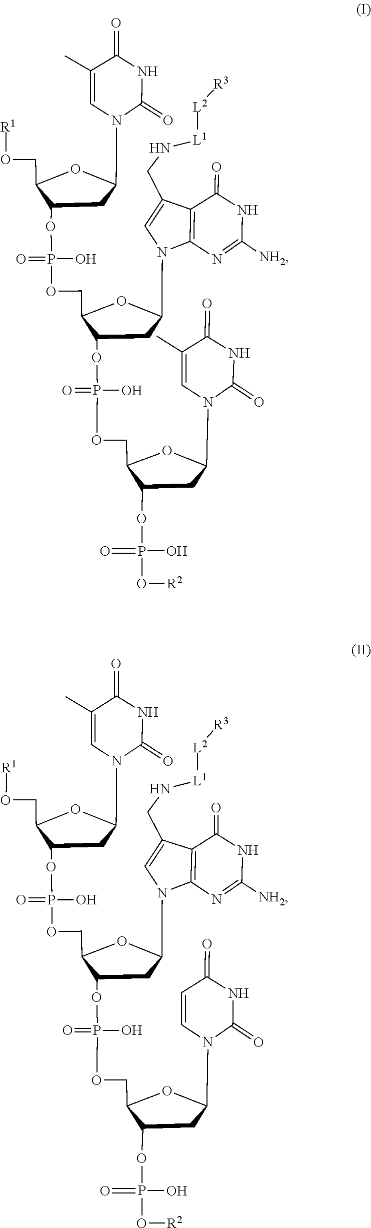

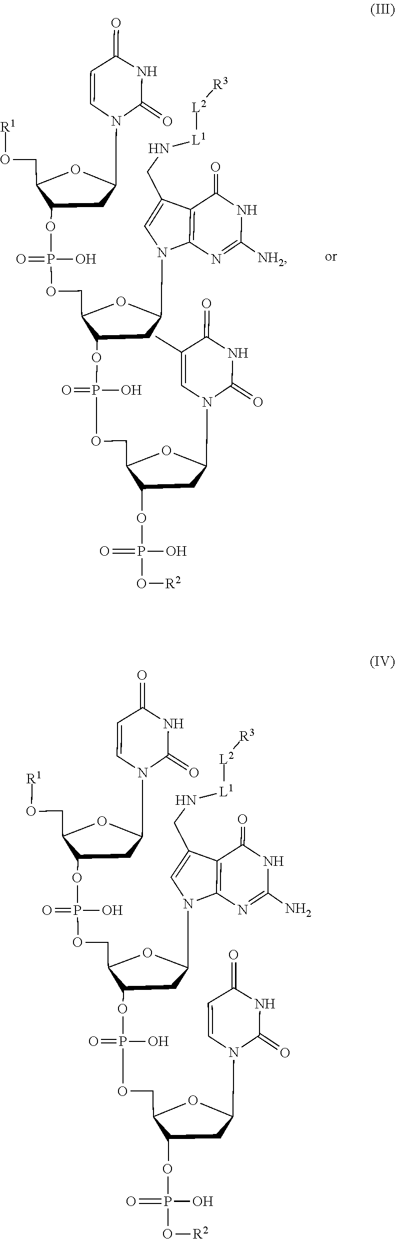

wherein
R¹ is hydrogen, a deoxynucleotide, or a first DNA sequence comprising the ₅′ of a DNA hairpin;
R² is hydrogen, a deoxynucleotide, or a second DNA sequence comprising the ₃′ of the DNA hairpin;
L¹ and L² are independently a bond, —O—, —S—, —NH—, —C(O)NH₂, —NHC(O)—, —C(O)O—, —OC(O)—, —S(O)₂NH₂, —NHS(O)₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene, a peptide linker, or a cleavable linker;
R³ is a detectable moiety, a biomolecular moiety, a therapeutic moiety, hydrogen, halogen, —CX^{3.1}₃, —CHX^{3.1}₂, —CH₂X^{3.1}, —CN, —SOₙ₁R^{3A}, —SOᵥ₁NR^{3B}R^{3C}, —NHNR^{3B}R^{3C}, —ONR^{3B}R^{3C}, —NHC(O)NHNR^{3B}R^{3C}, —NHC(O)NR^{3B}R^{3C}, —N(O)ₘ₁, —NR^{3B}R^{3C}, —C(O)R^{3D}, —C(O)OR^{3D}, —C(O)NR^{3B}R^{3C}, —OR^{3A}, —NR^{3B}SO₂R^{3A}, —NR^{3B}C(O)R^{3D}, —NR^{3B}C(O)OR^{3D}, —NR^{3B}OR^{3D}, —OCX^{3.1}₃, —OCHX^{3.1}₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3A}, R^{3B}, R^{3C} and R^{3D} are independently hydrogen, halogen, —CF₃, —CCl₃, —CBr₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3B} and R^{3C} substituents bonded to the same nitrogen atom may optionally be joined to form a substituted or unsubstituted heterocycloalkyl or substituted or unsubstituted heteroaryl; and
X^{3.1} is independently-Cl, —Br, —I or —F.

Embodiment 36: The DNA compound of embodiment 35, having the formula:

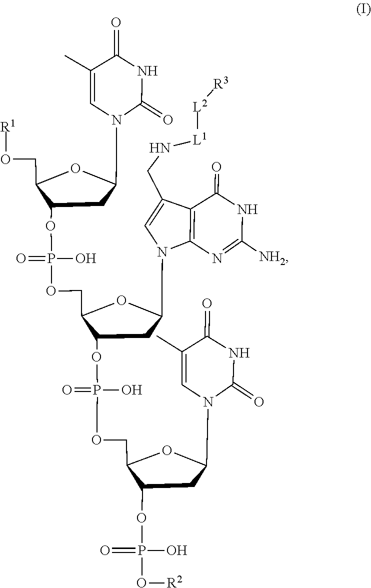

Embodiment 37: The DNA compound of embodiment 35, having the formula:

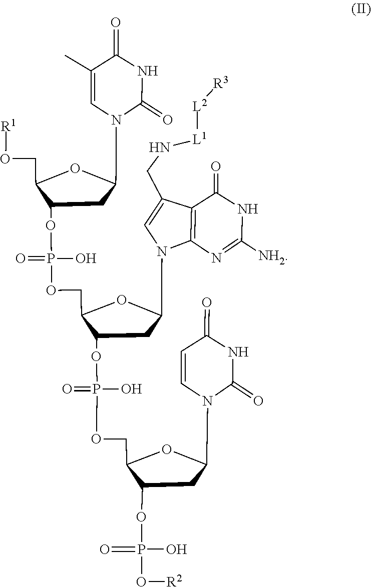

Embodiment 38: The DNA compound of embodiment 35, having the formula:

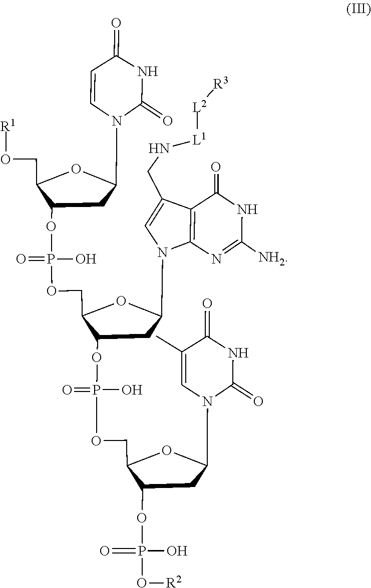

Embodiment 39: The DNA compound of embodiment 35, having the formula:

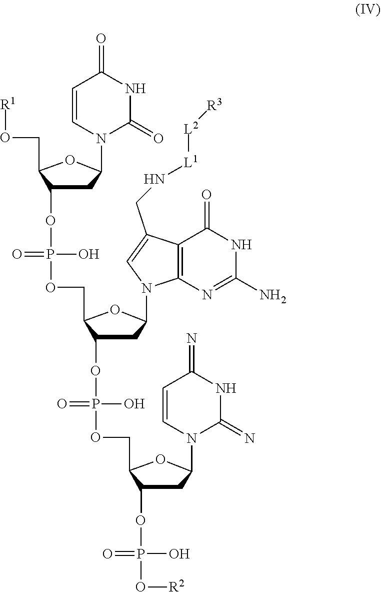

Embodiment 40: The DNA compound of any one of embodiments 35-39, wherein R¹ and R² are independently hydrogen.

Embodiment 41: The DNA compound of any one of embodiments 35-39, wherein R¹ and R² are independently a deoxynucleotide.

Embodiment 42: The DNA compound of embodiment 41, wherein R¹ and R² are independently a deoxyadenosine, deoxyguanosine, thymidine, deoxyuridine, or deoxycytidine.

Embodiment 43: The DNA compound of any one of embodiments 35-39, wherein R¹ and R² are independently a first DNA sequence comprising the 5′ of a DNA hairpin and a second DNA sequence comprising the 3′ of the DNA hairpin.

Embodiment 44: The DNA compound of one of embodiments 35-43, wherein L¹ is a bond or unsubstituted 2 to 12 membered heteroalkylene.

Embodiment 45: The DNA compound of one of embodiments 35-44, wherein L² is a bond or unsubstituted 2 to 12 membered heteroalkylene.

Embodiment 46: The DNA compound of one of embodiments 35-45, wherein L¹ or L² comprises a photo-cleavable site.

Embodiment 47: The DNA compound of one of embodiments 35-45, wherein L¹ or L² comprises a peptide linker.

Embodiment 48: The DNA method of embodiment 47, wherein said peptide linker comprises a protease cleavable site.

Embodiment 49: The DNA compound of embodiment 48, wherein said protease cleavable site is a matrix metalloprotease (MMP) cleavage site, a metalloprotease domain-containing (ADAM) metalloprotease cleavage site, a lysosomal cathepsin cleavage site (ALAL), or a legumain endopeptidase cleavage site (TANL).

Embodiment 50: The DNA compound of any one of embodiments 35-45, wherein L¹ comprises a pH sensitive cleavable site.

Embodiment 51: The DNA compound of any one of embodiments 35-50, wherein R³ is a detectable moiety.

Embodiment 52: The DNA compound of embodiment 51, wherein the detectable moiety is a fluorescent compound.

Embodiment 53: A cell comprising the DNA compound of any one of embodiments 35-52.

Embodiment 54: The cell of embodiment 53, further comprising a tRNA-guanine transglycosylase (TGT) enzyme.

Embodiment 55: A method of modifying a DNA molecule, the method comprising contacting the DNA molecule with a tRNA-guanine transglycosylase (TGT) enzyme and a PreQ1 derivative,

wherein the DNA molecule comprises a guanine nucleobase within a loop portion of a hairpin in the DNA molecule, and
wherein the PreQ1 derivative has the formula:

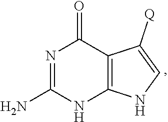

wherein Q is not hydrogen.

Embodiment 56: The method of embodiment 55, wherein the guanine nucleobase is in a YYGYYYY (SEQ ID NO: 103) sequence within the loop portion of the hairpin in the DNA molecule

Embodiment 57: The method of embodiment 56, wherein the sequence within the loop portion of the hairpin is YTGTYYY (SEQ ID NO:104), YTGTCCY (SEQ ID NO:105), YTGTYCC (SEQ ID NO:106), YUGUYYY (SEQ ID NO:107), YUGTYYY (SEQ ID NO:108), or YTGUYYY (SEQ ID NO:109).

Embodiment 58: The method of embodiment 57, wherein the sequence within the loop portion of the hairpin is YTGTCCY (SEQ ID NO:105) or YTGTYCC (SEQ ID NO:106).

Embodiment 59: The method of any one of embodiments 55-58, wherein the DNA molecule is between about 15 nucleotides to about 30,000 nucleotides in length.

Embodiment 60: The method of any one of embodiments 55-59, wherein the DNA molecule is between about 15 nucleotides to about 150 nucleotides in length.

Embodiment 61: The method of any one of embodiments 55-60, wherein the DNA molecule is a gene, a cDNA, a non-coding DNA, an extracellular DNA (eDNA), an antisense oligonucleotide, a barcode, a probe, or a primer.

Embodiment 62: The method of any one of embodiments 55-61, wherein the DNA molecule, TGT enzyme, and the PreQ1 derivative are added to the reaction mixture simultaneously.

Embodiment 63: The method of any one of embodiments 55-61, wherein the DNA molecule and the PreQ1 derivative are added to the reaction mixture prior to adding the TGT enzyme.

Embodiment 64: The method of any one of embodiments 55-63, wherein Q is -L¹-L²-R³;
L¹ and L² are independently a bond, —O—, —S—, —NH—, —C(O)NH₂, —NHC(O)—, —C(O)O—, —OC(O)—, —S(O)₂NH₂, —NHS(O)₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene, a peptide linker, or a cleavable linker;
R³ is a detectable moiety, a biomolecular moiety, a therapeutic moiety, hydrogen, halogen, —CX^{3.1}₃, —CHX^{3.1}₂, —CH₂X^{3.1}, —CN, —SOₙ₁R^{3A}, —SOᵥ₁NR^{3B}R^{3C}, —NHNR^{3B}R^{3C}, —ONR^{3B}R^{3C}, —NHC(O)NHNR^{3B}R^{3C}, —NHC(O)NR^{3B}R^{3C}, —N(O)ₘ₁, —NR^{3B}R^{3C}, —C(O)R^{3D}, —C(O)OR^{3D}, —C(O)NR^{3B}R^{3C}, —OR^{3A}, —NR^{3B}SO₂R^{3A}, —NR^{3B}C(O)R^{3D}, —NR^{3B}C(O)OR^{3D}, —NR^{3B}OR^{3D}, —OCX^{3.1}₃, —OCHX^{3.1}₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3A}, R^{3B}, R^{3C} and R^{3D} are independently hydrogen, halogen, —CF₃, —CCl₃, —CBr₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3B} and R^{3C} substituents bonded to the same nitrogen atom may optionally be joined to form a substituted or unsubstituted heterocycloalkyl or substituted or unsubstituted heteroaryl; and
X^{3.1} is independently-Cl, —Br, —I or —F.

Embodiment 65: The method of embodiment 64, wherein L¹ or L² comprises a photo-cleavable site.

Embodiment 66: The method of embodiment 64, wherein L¹ or L² comprises a peptide linker.

Embodiment 67: The method of any embodiment 66, wherein said peptide linker comprises a protease cleavable site.

Embodiment 68: The method of embodiment 68, wherein said protease cleavable site is a matrix metalloprotease (MMP) cleavage site, a metalloprotease domain-containing (ADAM) metalloprotease cleavage site, a lysosomal cathepsin cleavage site (ALAL), or a legumain endopeptidase cleavage site (TANL).

Embodiment 69: The method of embodiment 64, wherein L¹ comprises a pH sensitive cleavable site.

Embodiment 70: The method of any one of embodiments 64-69, wherein R³ is a detectable moiety.

Embodiment 71: The method of embodiment 70, wherein the detectable moiety is a fluorescent moiety.

Embodiment 72: A method of substituting a guanine nucleobase within a DNA molecule with a PreQ1 derivative, the method comprising contacting the DNA molecule and PreQ1 derivative with a tRNA-guanine transglycosylase (TGT) enzyme,

wherein the guanine nucleobase is within a loop portion of a hairpin in the DNA molecule, and
wherein the PreQ1 derivative has the formula:

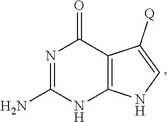

wherein Q is not hydrogen.

Embodiment 73: The method of embodiment 72, wherein the guanine nucleobase is in a YYGYYYY (SEQ ID NO: 103) sequence within the loop portion of the hairpin in the DNA molecule.

Embodiment 74: The method of embodiment 73, wherein the sequence within the loop portion of the hairpin is YTGTCCY (SEQ ID NO:105), YTGTYCC (SEQ ID NO:106), YUGUYYY (SEQ ID NO:107), YUGTYYY (SEQ ID NO:108) or YTGUYYY (SEQ ID NO:109).

Embodiment 75: The method of embodiment 74, wherein the sequence within the loop portion of the hairpin is YTGTCCY (SEQ ID NO:105) or YTGTYCC (SEQ ID NO:106).

Embodiment 76: The method of any of embodiments 72-75, wherein the DNA molecule is between about 15 nucleotides to about 30,000 nucleotides in length.

Embodiment 77: The method of any of embodiments 72-76, wherein the DNA molecule is between about 15 nucleotides to about 150 nucleotides in length.

Embodiment 78: The method of any one of embodiments 72-77, wherein the DNA molecule is a gene, a cDNA, a non-coding DNA, an extracellular DNA (eDNA), an antisense oligonucleotide, a barcode, a probe, or a primer.

Embodiment 79: The method of any one of embodiments 72-78, wherein the DNA molecule, TGT enzyme, and the PreQ1 derivative are added to the reaction mixture simultaneously.

Embodiment 80: The method of any one of embodiments 72-78, wherein the DNA molecule and the PreQ1 derivative are added to the reaction mixture prior to adding the TGT enzyme.

Embodiment 81: The method of any one of embodiments 72-80, wherein Q is -L¹-L²-R³;
L¹ and L² are independently a bond, —O—, —S—, —NH—, —C(O)NH₂, —NHC(O)—, —C(O)O—, —OC(O)—, —S(O)₂NH₂, —NHS(O)₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene, a peptide linker, or a cleavable linker;
R³ is a detectable moiety, a biomolecular moiety, a therapeutic moiety, hydrogen, halogen, —CX^{3.1}₃, —CHX^{3.1}₂, —CH₂X^{3.1}, —CN, —SOₙ₁R^{3A}, —SOᵥ₁NR^{3B}R^{3C}, —NHNR^{3B}R^{3C}, —ONR^{3B}R^{3C}, —NHC(O)NHNR^{3B}R^{3C}, —NHC(O)NR^{3B}R^{3C}, —N(O)ₘ₁, —NR^{3B}R^{3C}, —C(O)R^{3D}, —C(O)OR^{3D}, —C(O)NR^{3B}R^{3C}, —OR^{3A}, —NR^{3B}SO₂R^{3A}, —NR^{3B}C(O)R^{3D}, —NR^{3B}C(O)OR^{3D}, —NR^{3B}OR^{3D}, —OCX^{3.1} ₃—OCHX^{3.1}₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3A}, R^{3B}, R^{3C} and R^{3D} are independently hydrogen, halogen, —CF₃, —CCl₃, —CBr₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3B} and R^{3C} substituents bonded to the same nitrogen atom may optionally be joined to form a substituted or unsubstituted heterocycloalkyl or substituted or unsubstituted heteroaryl; and
X^{3.1} is independently-Cl, —Br, —I or —F.

Embodiment 82: The method of embodiment 81, wherein L¹ or L² comprises a photo-cleavable site.

Embodiment 83: The method of embodiment 81, wherein L¹ or L² comprises a peptide linker.

Embodiment 84: The method of any embodiment 83, wherein said peptide linker comprises a protease cleavable site.

Embodiment 85: The method of embodiment 84, wherein said protease cleavable site is a matrix metalloprotease (MMP) cleavage site, a metalloprotease domain-containing (ADAM) metalloprotease cleavage site, a lysosomal cathepsin cleavage site (ALAL), or a legumain endopeptidase cleavage site (TANL).

Embodiment 86: The method of embodiment 81, wherein L¹ comprises a pH sensitive cleavable site.

Embodiment 87: The method of any one of embodiments 81-86, wherein R³ is a detectable moiety.

Embodiment 88: The method of embodiment 87, wherein the detectable moiety is a fluorescent moiety.

Embodiment 89: A DNA compound having the formula:

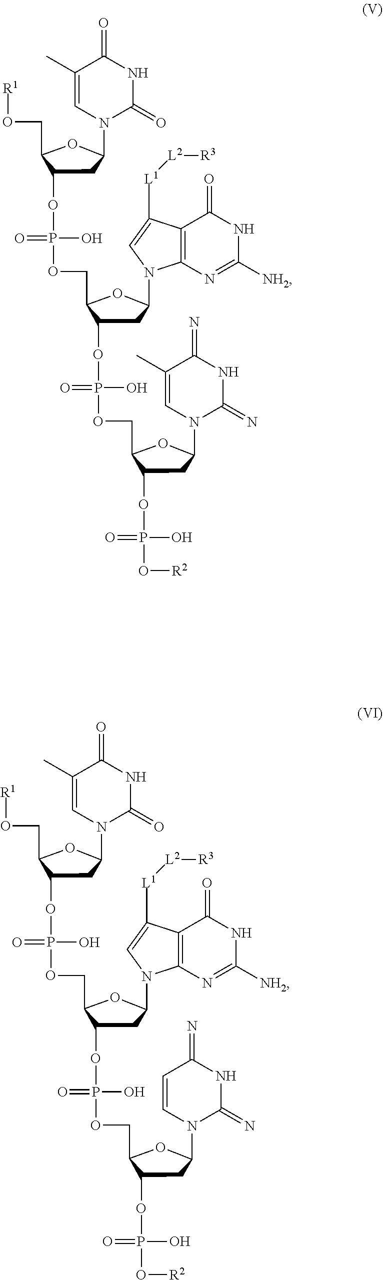

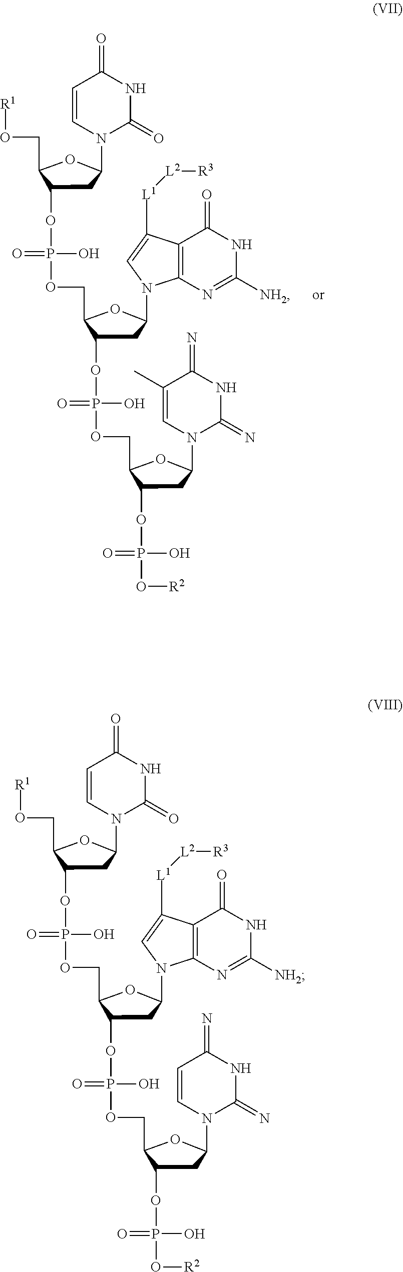

(VIII); wherein
R¹ is hydrogen, a deoxynucleotide, or a first DNA sequence comprising the 5′ of a DNA hairpin;
R² is hydrogen, a deoxynucleotide, or a second DNA sequence comprising the 3′ of the DNA hairpin;
L¹ and L² are independently a bond, —O—, —S—, —NH—, —C(O)NH₂, —NHC(O)—, —C(O)O—, —OC(O)—, —S(O)₂NH₂, —NHS(O)₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene, a peptide linker, or a cleavable linker;
R³ is a detectable moiety, a biomolecular moiety, a therapeutic moiety, hydrogen, halogen, —CX^{3.1}₃, —CHX^{3.1}₂, —CH₂X^{3.1}, —CN, —SOₙ₁R^{3A}, —SOᵥ₁NR^{3B}R^{3C}, —NHNR^{3B}R^{3C}, —ONR^{3B}R^{3C}, —NHC(O)NHNR^{3B}R^{3C}, —NHC(O)NR^{3B}R^{3C}, —N(O)ₘ₁, —NR^{3B}R^{3C}, —C(O)R^{3D}, C(O)OR^{3D}, —C(O)NR^{3B}R^{3C}, —OR^{3A}, —NR^{3B}SO₂R^{3A},—NR^{3B}C(O)R^{3D}, —NR^{3B}C(O)OR^{3D}, —NR^{3B}OR^{3D}, —OCX^{3.1}₃, —OCHX^{3.1} ₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3A}, R^{3B}, R^{3C} and R^{3D} are independently hydrogen, halogen, —CF₃, —CCl₃, —CBr₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3B} and R^{3C} substituents bonded to the same nitrogen atom may optionally be joined to form a substituted or unsubstituted heterocycloalkyl or substituted or unsubstituted heteroaryl; and
X^{3.1} is independently-Cl, —Br, —I or —F.

Embodiment 90: The DNA compound of embodiment 89, having the formula:

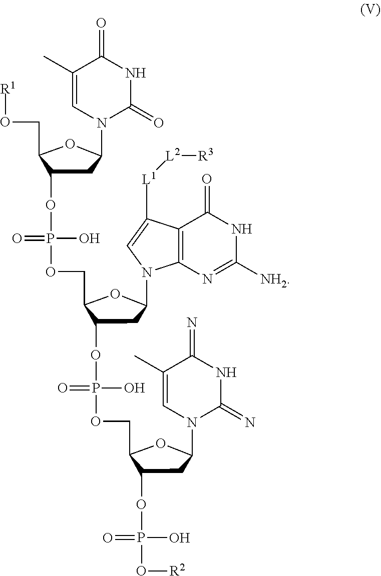

Embodiment 91: The DNA compound of embodiment 89, having the formula:

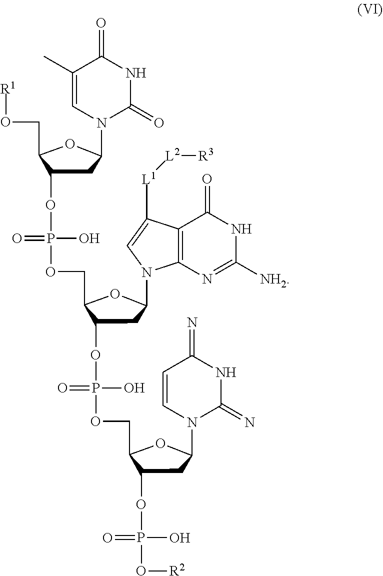

Embodiment 92: The DNA compound of embodiment 89, having the formula

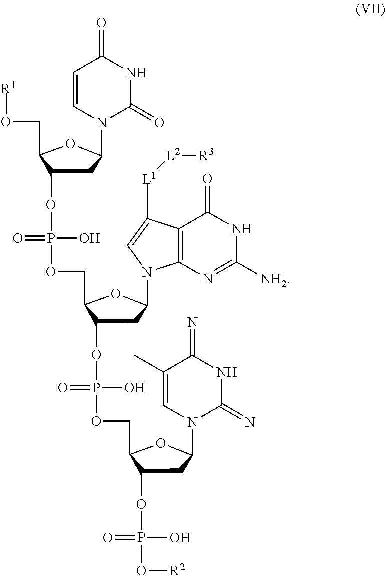

Embodiment 93: The DNA compound of embodiment 89, having the formula:

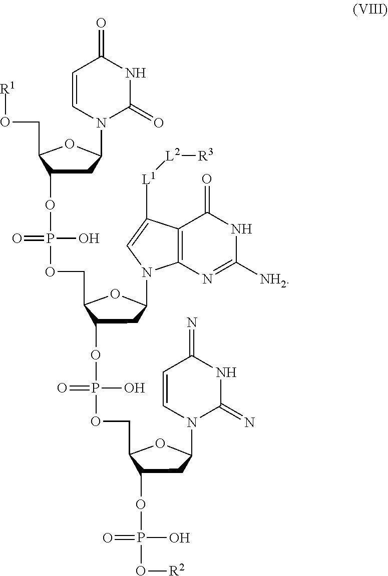

Embodiment 94: The DNA compound of any one of embodiments 89-93, wherein R¹ and R² are independently hydrogen.

Embodiment 95: The DNA compound of any one of embodiments 89-93, wherein R¹ and R² are independently a deoxynucleotide.

Embodiment 96: The DNA compound of any one of embodiments 89-93, wherein R¹ and R² are independently a deoxyadenosine, deoxyguanosine, thymidine, deoxyuridine, or deoxycytidine.

Embodiment 97: The DNA compound of any one of embodiments any one of embodiments 89-93, wherein R¹ and R² are independently a first DNA sequence comprising the 5′ of a DNA hairpin and a second DNA sequence comprising the 3′ of the DNA hairpin.

Embodiment 98: The DNA compound of one of embodiments 89-97, wherein L¹ is a bond or unsubstituted 2 to 12 membered heteroalkylene.

Embodiment 99: The DNA compound of one of embodiments 89-98, wherein L² is a bond or unsubstituted 2 to 12 membered heteroalkylene.

Embodiment 100: The DNA compound of one of embodiments 89-99, wherein L¹ or L² comprises a photo-cleavable site.

Embodiment 101: The DNA compound of one of embodiments 89-99, wherein L¹ or L² comprises a peptide linker.

Embodiment 102: The DNA method of embodiment 101, wherein said peptide linker comprises a protease cleavable site.

Embodiment 103: The DNA compound of embodiment 102, wherein said protease cleavable site is a matrix metalloprotease (MMP) cleavage site, a metalloprotease domain-containing (ADAM) metalloprotease cleavage site, a lysosomal cathepsin cleavage site (ALAL), or a legumain endopeptidase cleavage site (TANL).

Embodiment 104: The DNA compound of any one of embodiments 89-99, wherein L¹ or L¹ comprises a pH sensitive cleavable site.

Embodiment 105: The DNA compound of any one of embodiments 89-104, wherein R³ is a detectable moiety.

Embodiment 106: The DNA compound of embodiment 105, wherein the detectable moiety is a fluorescent compound.

Embodiment 107: A cell comprising the DNA compound of any one of embodiments 89-106.

Embodiment 108: The cell of embodiment 107, further comprising a tRNA-guanine transglycosylase (TGT) enzyme.

### EXAMPLES

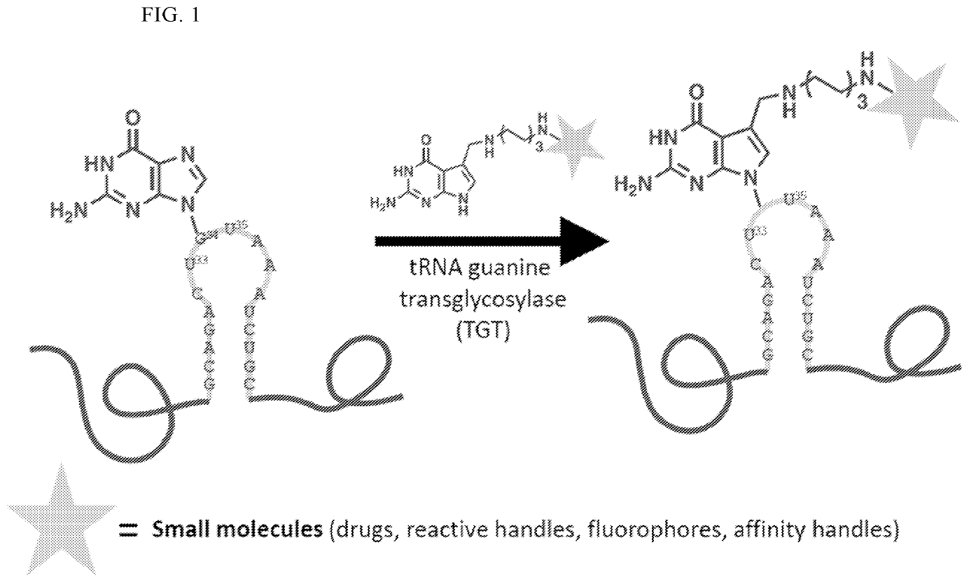

Previous work from our group has established RNA-TAG, an RNA modification strategy using tRNA guanine transglycosylase (TGT). TGT catalyzes the exchange of guanine for 7-deazaguanine derivatives on its native substrate, the anticodon loop of queuine-cognate tRNAs, however, it has been shown that the minimum recognition sequence for TGT is a short 17 nt hairpin with a uridine flanked target guanine in its loop. In RNA-TAG, e. coli TGT recognizes this minimal hairpin loop incorporated into an RNA of interest and catalyzes the exchange of the target guanine for synthetically modified PreQ1 derivatives bearing a reporter, thus allowing the downstream application or investigation of the RNA of interest. Previously, TGT was deemed incapable of modifying native DNA substrates, and it was thought that nucleobase modifications were required for efficient recognition of the nucleic acid substrate. Preliminary experiments using carefully designed DNA and RNA mutant oligonucleotide substrates led us to hypothesize that the inability of TGT to turn over the analogous DNA hairpin was likely due to steric limitations rather than electronic. Through iterative testing of rationally designed DNA hairpins we uncovered a series of several 17 nucleotide DNA hairpins that are efficiently recognized by the enzyme. A complete reaction scheme is shown in FIG. 1. This 17 nucleotide recognition sequence can either be inserted internally or appended to either end of ssDNA of interest allowing for installation of a functional handle. Additionally, the versatility of TGT and its tolerance toward substrate modification allows for the one step incorporation of all reporters tested, varying in size and overall charge.

Applications for this technology include DNA scaffolding, barcoding, immobilization, conjugation, and nucleic acid detection such as single molecule FISH and northern/southern blotting, among others. Currently, there are many strategies for single molecule FISH many of which serve to amplify and clarify signal. These quantitative detection strategies rely on each probe containing a defined number of reporters and require an efficient, high yielding probe set synthesis. In this work we demonstrate the ability of DNA-TAG to effectively generate fluorescent tiled antisense oligo probe sets. Additionally, we demonstrate the ability of our technology to prepare fluorescent antisense probes for the detection of RNA via northern blot.

### Results and Discussion

The efficiency and reliability with which TGT inserts functional PreQ1 probes into an RNA of interest coupled with the published mechanism of TGT catalysis alludes to the possibility that the enzyme could similarly recognize DNA substrates. While it was previously reported that TGT does not recognize the wild type DNA substrate analogous to the minimal 17 nt RNA hairpin substrate, when all “dT” nucleotides were replaced with “dU” the enzyme was able to modify the DNA substrate. With this information we designed a series of experiments to assess the limitations of TGT toward DNA substrates, limiting the number of dT to dU mutations to only those that are part of the determined minimal recognition element in RNA, “UGU”. For all of the preliminary experiments, the DNA oligos tested used the extended stem DNA analog of the ECYA1 sequence (GGGAGCAGACYGYAAATCTGCTCCC) (SEQ ID NO: 102) where the loop sequence is underlined, and the bolded residues are considered the “minimum recognition element”. Oligos containing the following minimal recognition element mutations were tested for TGT activity using a PreQ1-biotin probe: dTdGdT, dUdGdU, dUdGdT, dTdGdU. While dTdGdT and dUdGdT showed no appreciable turn over by the enzyme, both dUdGdU and dTdGdU showed comparable turn over to the wildtype RNA substrate. The single mutation of the minimal recognition element to dTdGdU allows for recognition and efficient turnover of a DNA substrate by TGT. The presence (or absence) and efficiency of labeling is evidenced via denaturing urea PAGE by the upward shift of oligo mobility due to an increase in molecular weight and intensity of signal, respectively (FIGS. 2A-2D). This expands on previous knowledge which only assessed the turnover of a substrate where all dT nucleotides were replaced with dU. This promising information led to the next question: can TGT turn over an RNA substrate where all of the U nucleotides are replaced with 5-meU (“rT”)? This substrate had a similar turnover to that of the wildtype RNA hairpin as evidenced by urea PAGE gel shift and intensity (FIGS. 2A-2D).

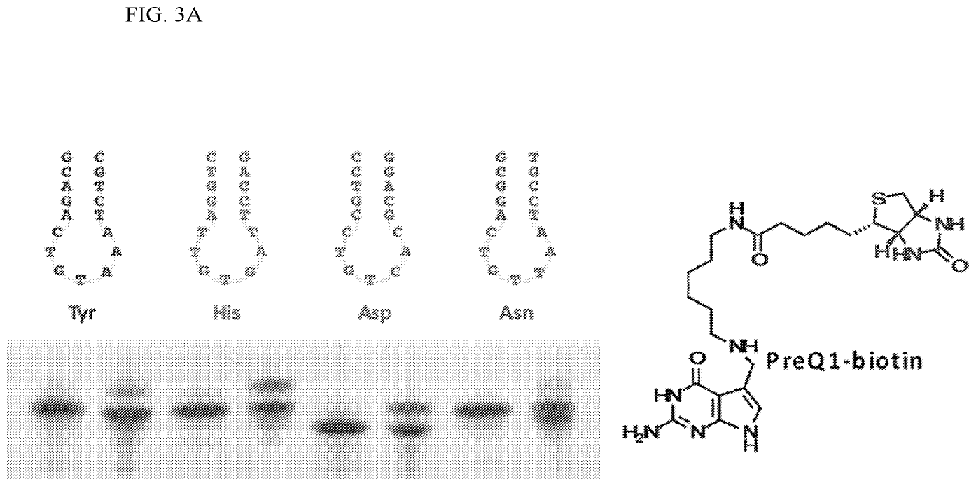

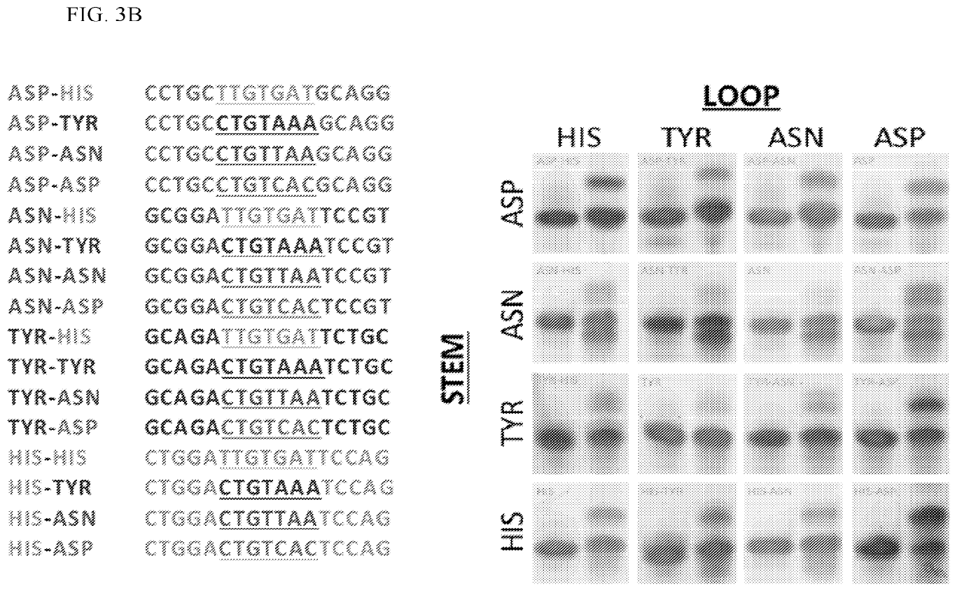

Convinced at this point that TGTs inability to turnover DNA ECYA1 was merely steric and could therefore be overcome by finding the right DNA substrate. We reasoned that by altering bases in both the stem and loop of the dECYA1 sequence, the conformation would alter just enough to sterically allow TGT to act on it. The ECYA1 hairpin is derived from the anticodon loop of tyrosine tRNA. However, TGT has 3 other cognate tRNA substrates: histidine, aspartate, and asparagine. While they all have the same “UGU” minimal recognition element in their anticodon loops, the rest of the hairpin sequence varies. Both the histidine and aspartate derived 17 nucleotide DNA hairpins showed appreciable increase in TGT activity compared to their tyrosine based counterpart, increasing from ˜-5% to ˜40% labeled product as evidenced by urea PAGE gel shift and intensity (FIG. 3A).

To determine the importance of the stems and/or loops for each of these substrates, we designed composite hairpins, swapping the stems and loops for all four DNA analogs. The results of this experiment suggest that while both the stem and the loop sequences contribute to the ability of TGT to turnover a given substrate, the loop sequence plays the more critical role in substrate determination. Of these substrates, the His(stem)-Asp(loop) was labeled with the highest efficiency, reaching over 50%, as evidenced by urea PAGE gel shift and intensity (FIG. 3B).

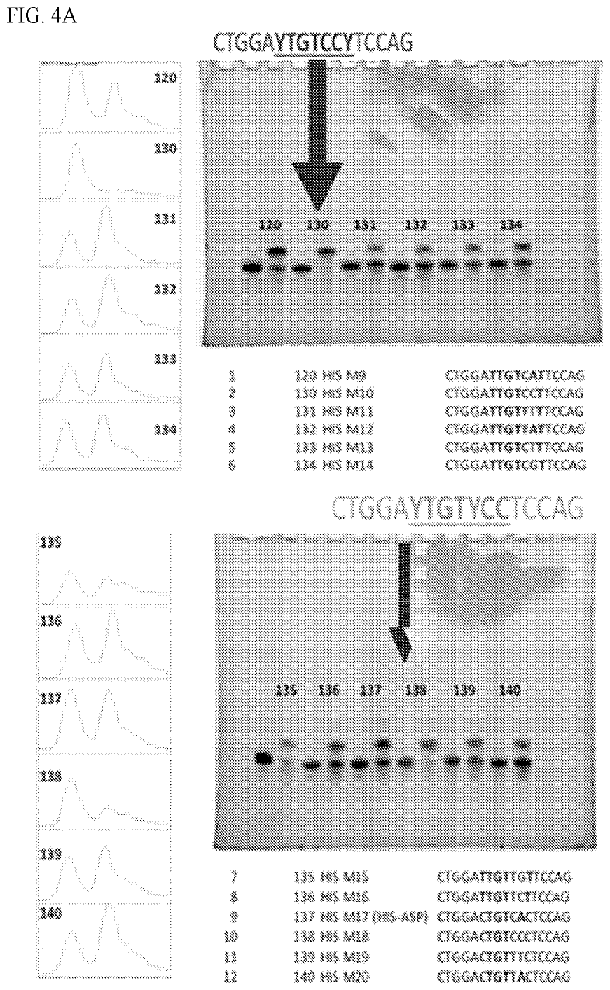

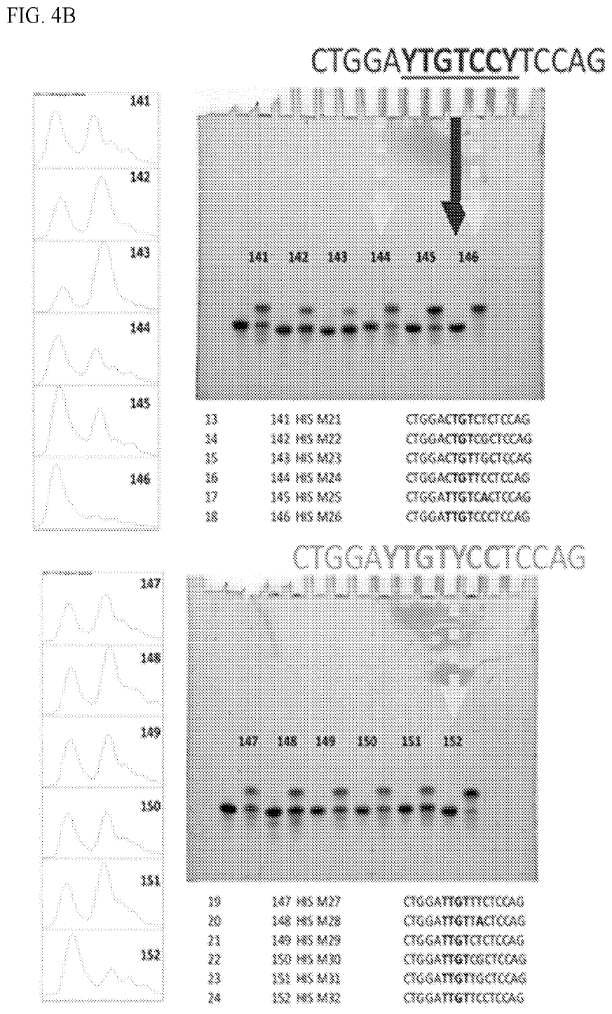

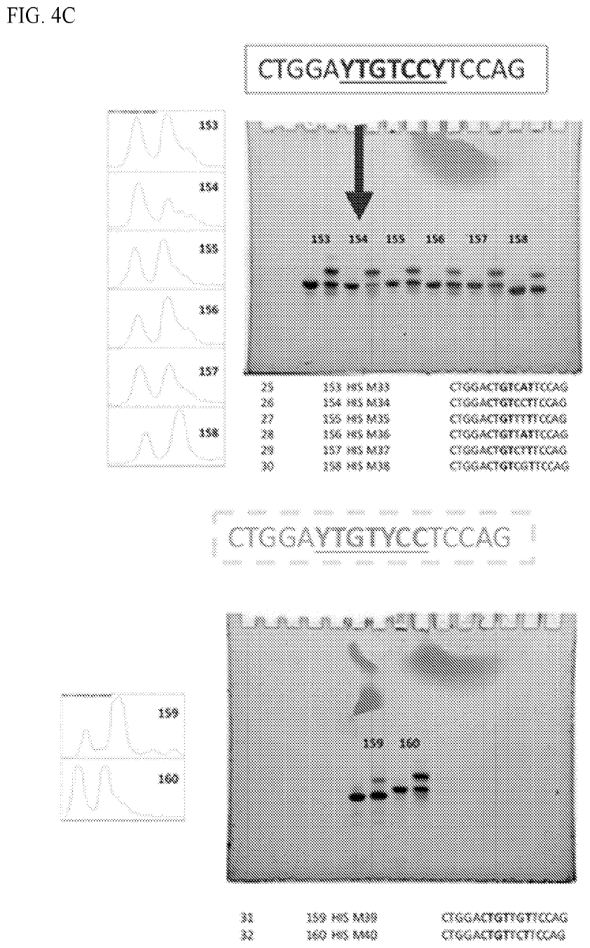

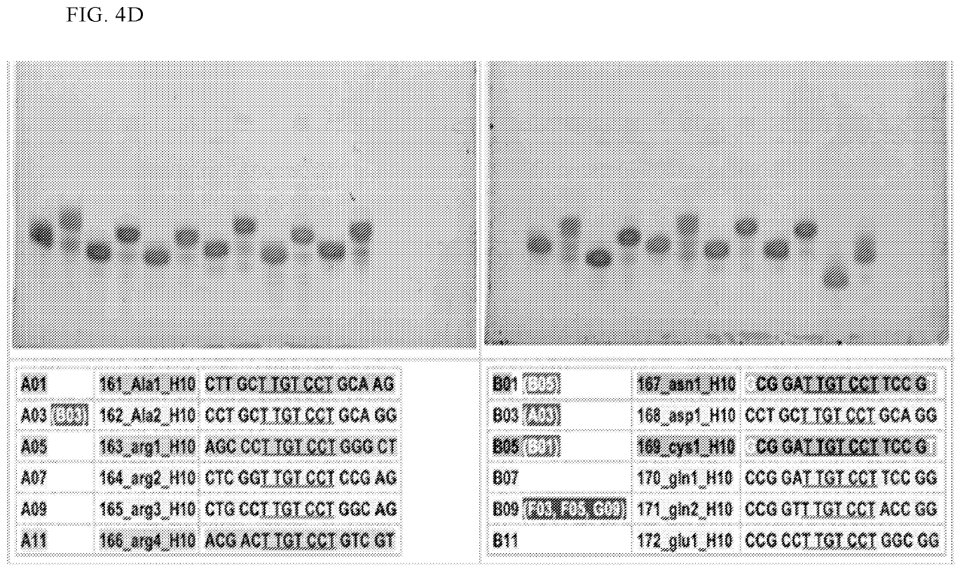

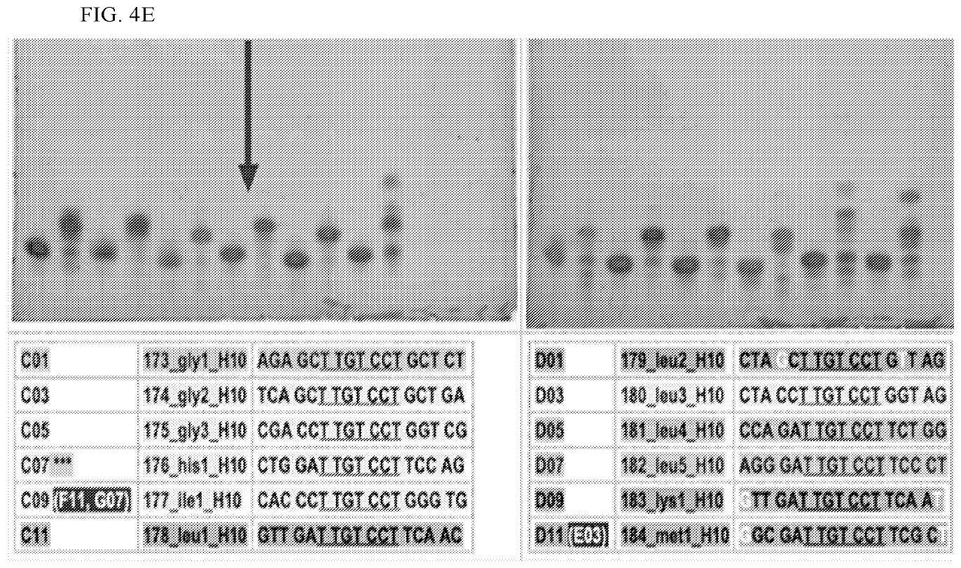

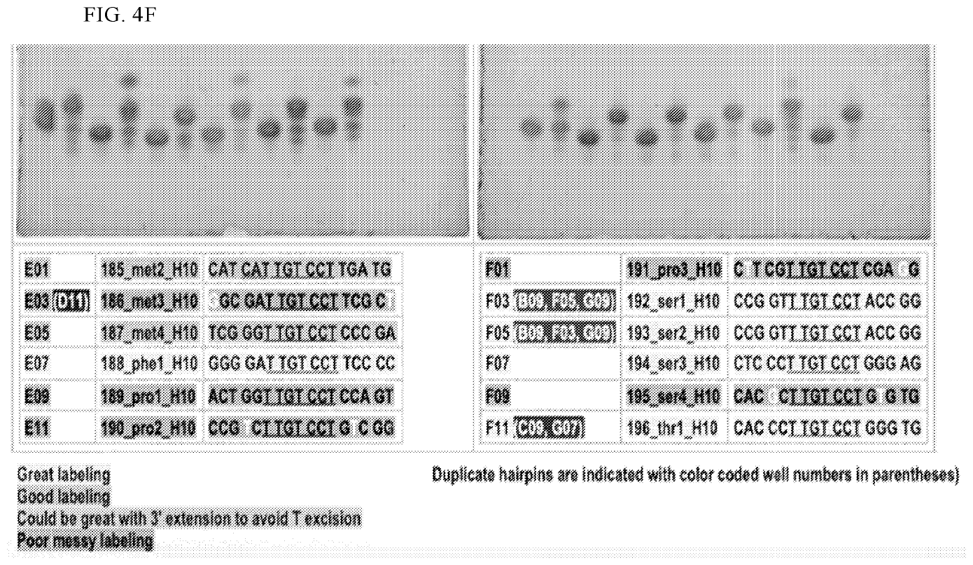

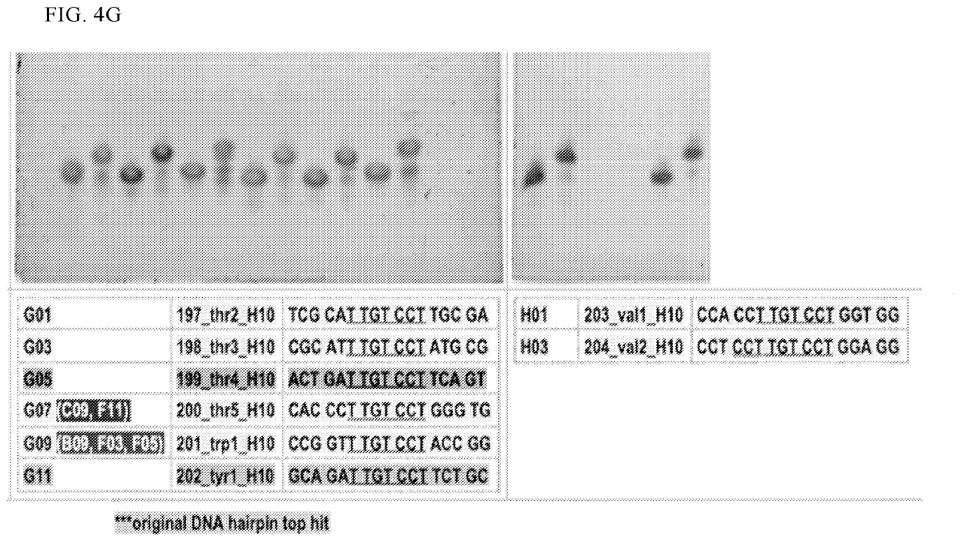

A variety of loop mutants of the HIS stem were tested. These experiments confirmed the importance of the loop, finally leading to a minimal substrate loop consensus of either YTGTCCY (SEQ ID NO:105) or YTGTYCC (SEQ ID NO:106) where Y contains smaller pyrimidine bases (C or T), confirming our suspicion that sterics may play a role in substrate recognition (FIGS. 4A-4C). Using the loop of one of the top candidates, a series of other stems were tested to determine the effect of stem sequence, revealing that indeed, with a consensus loop, the stem had less effect, as evidenced by urea PAGE gel shift and intensity (FIGS. 4D-4G).

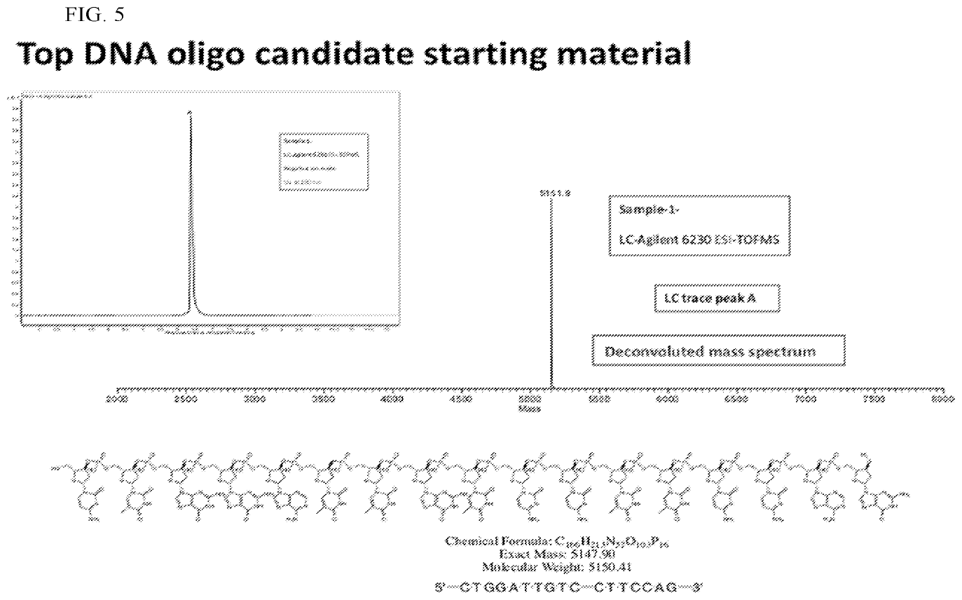

A representative hairpin with the sequence CTGGATTGTCCTTCCAG (SEQ ID NO:21) was further investigated. LCMS analysis of the PreQ1-biotin labeled sequence confirmed the efficient labeling observed via gel shift. The LC trace shows a major peak containing the biotin labeled hairpin as confirmed by mass spectrometry (FIG. 5).

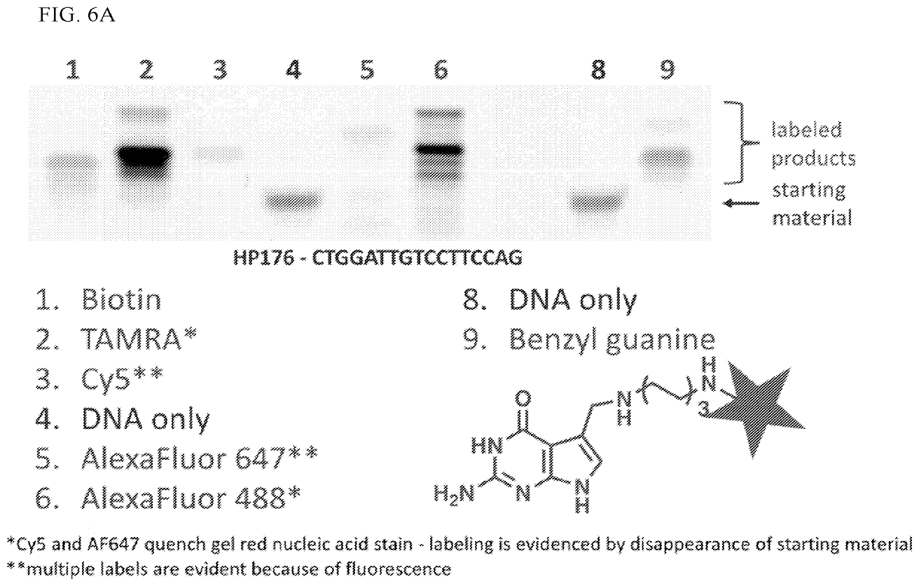

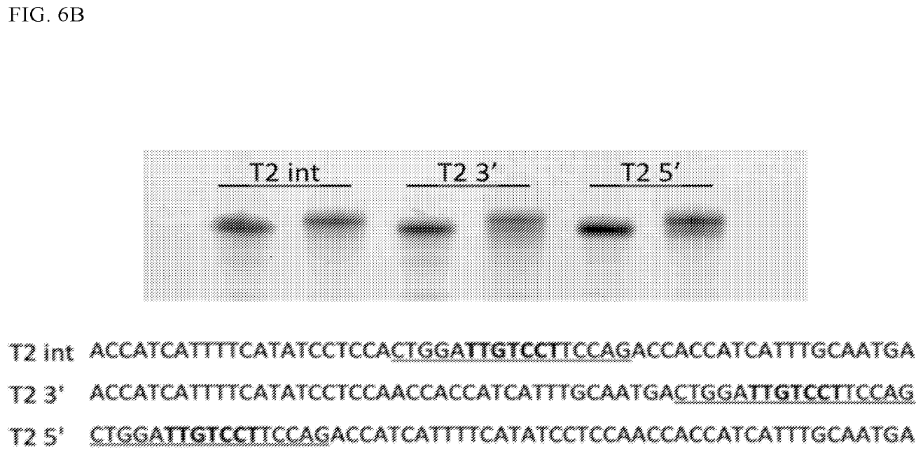

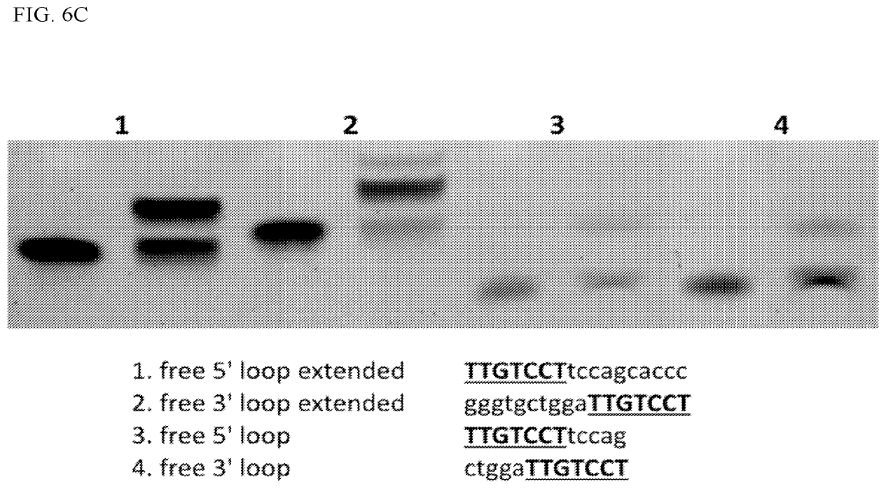

As with RNA, TGT is able to modify ssDNA with a wide variety of functional probes. Quantitative labeling of the representative DNA hairpin substrate was demonstrated by urea PAGE gel shift assays with PreQ1-biotin, TAMRA, Cy5, Alexa Fluor 647, and Alex Fluor 488 (FIG. 6A). To determine whether TGT could still recognize the DNA hairpin when it was appended to a longer ssDNA of interest, we made 59 mers of 5′, 3′, and internal conjugates and tested the extent of their labeling. All three hairpin adducts were efficiently labeled using the PreQ1-biotin probe, demonstrated by urea PAGE gel shift (FIG. 6B). TGT is able to recognize and efficiently modify the loop sequence at the 3′ end of a ssDNA oligo without the presence of a hairpin as evidenced by a shift on denaturing urea PAGE. The loop sequence at the 5′ end is also recognized and modified but to a lesser extent and longer oligos are more efficiently labeled than shorter oligos (FIG. 6C).

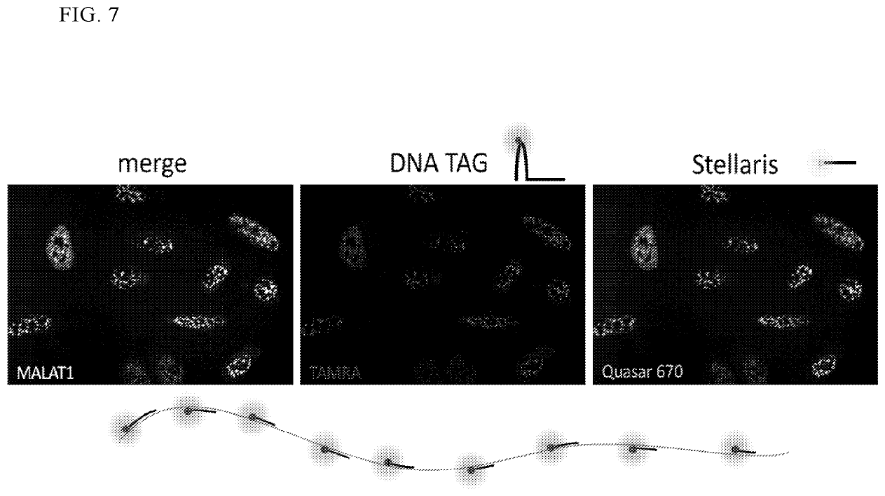

To demonstrate an application of the DNA TAG labeling strategy we performed RNA FISH experiments in U2OS cells. Probe sequences were designed against the long non coding RNA MALAT1 using the Stellaris probe designer. Probe sets of 48 unique oligos containing the DNA-TAG hairpin were fluorescently labeled using TGT and PreQ1-C6-TAMRA. U2OS cells were treated with a DNA-TAG TAMRA labeled probe set, a ready ship probe set from stellaris, and a combination of the two. Fluorescent confocal microscopy was used to confirm the ability of the TGT labeled probe set to effectively hybridize with MALAT 1 and colocalize with the commercial probe set as is cleary seen by presence and overlap of fluorescent puncta (FIG. 7).

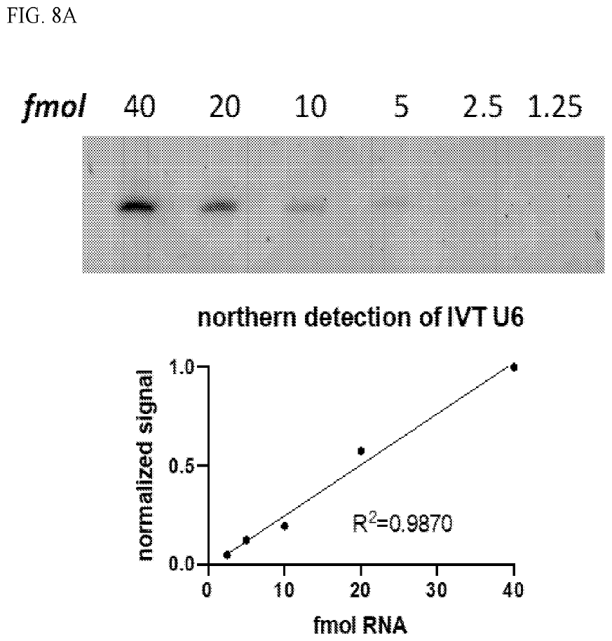

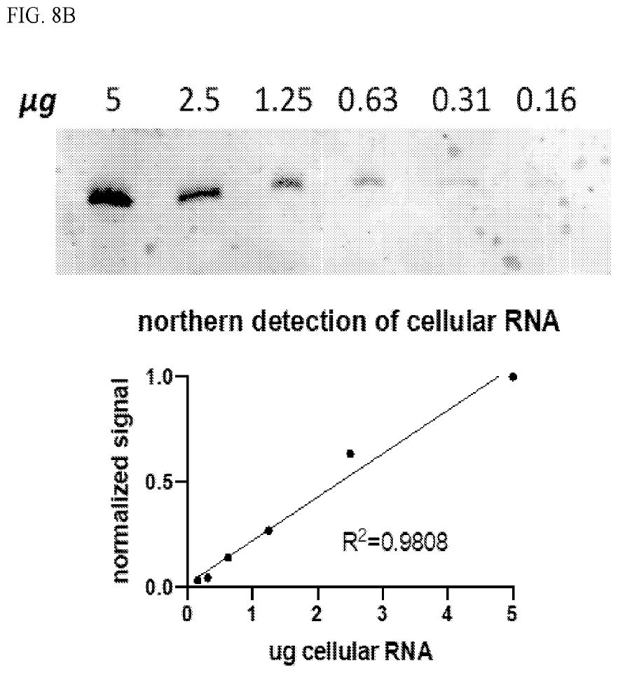

Additionally, we demonstrated the ability to detect RNA species via northern blot using DNA-TAG labeled antisense probes. A template for the small nuclear U6 RNA was PCR amplified from U20S DNA extracts, in vitro transcribed, and detected via northern blot after transfer from a urea PAGE gel. Similarly, native U6 RNA was detected from U20S cellular extracts. Both the IVT and cellular RNAs were detected in a dose dependent manner as can be seen in the dose dependent increase in fluorescent signal from the TGT labeled U6 antisense probe (FIGS. 8A-8B).

### CONCLUSIONS

Beyond being central to life, nucleic acids have proven to be an invaluable tool for various applications ranging from critical therapeutics to basic scientific research. Harnessing the unparalleled programmability and versatility afforded by these biological macromolecules requires tools that install functional handles or facilitate detection. Increasing the versatility and diversity of these tools is critical to the further advancement of relevant fields. Here we introduce a technology that allows for the enzymatic insertion of a variety of functional small molecules into ssDNA substrates of interest for downstream applications. This system is compatible for both internal and end DNA modifications and tolerant of a variety of small molecule substrates. Additionally, this technology allows an inexpensive and straightforward method by which researchers can quickly label several DNA oligos, either simultaneously or in parallel, in a single step and with a short spin column purification.

INFORMAL SEQUENCE LISTING
SEQ ID NO: 1
GGGAGCAGACTGTAAATCTGCTCCC
SEQ ID NO: 2
GGGAGCAGACUGUAAATCTGCTCCC
SEQ ID NO: 3
GGGAGCAGACTGUAAATCTGCTCCC
SEQ ID NO: 4
GGGAGCAGACUGTAAATCTGCTCCC
SEQ ID NO: 5
GGGAGCAGACUGUAAAUCUGCUCCC
SEQ ID NO: 6
GCAGACTGTAAATCTGC
SEQ ID NO: 7
CTGGATTGTGATTCCAG
SEQ ID NO: 8
CCTGCCTGTCACGCAGG
SEQ ID NO: 9
GCGGACTGTTAATCCGT
SEQ ID NO: 10
CCTGCTTGTGATGCAGG
SEQ ID NO: 11
CCTGCCTGTAAAGCAGG
SEQ ID NO: 12
CCTGCCTGTTAAGCAGG
SEQ ID NO: 13
GCGGATTGTGATTCCGT
SEQ ID NO: 14
GCGGACTGTAAATCCGT
SEQ ID NO: 15
GCGGACTGTCACTCCGT
SEQ ID NO: 16
GCAGATTGTGATTCTGC
SEQ ID NO: 17
GCAGACTGTTAATCTGC
SEQ ID NO: 18
GCAGACTGTCACTCTGC
SEQ ID NO: 19
CTGGAYTGTCCYTCCAG
SEQ ID NO: 20
CTGGATTGTCATTCCAG
SEQ ID NO: 21
CTGGATTGTCCTTCCAG
SEQ ID NO: 22
CTGGATTGTTTTTCCAG
SEQ ID NO: 23
CTGGATTGTTATTCCAG
SEQ ID NO: 24
CTGGATTGTCTTTCCAG
SEQ ID NO: 25
CTGGATTGTCGTTCCAG
SEQ ID NO: 26
CTGGAYTGTYCCTCCAG
SEQ ID NO: 27
CTGGATTGTTGTTCCAG
SEQ ID NO: 28
CTGGATTGTTCTTCCAG
SEQ ID NO: 29
CTGGACTGTCCCTCCAG
SEQ ID NO: 30
CTGGACTGTTTCTCCAG
SEQ ID NO: 31
CTGGACTGTTACTCCAG
SEQ ID NO: 32
CTGGACTGTCTCTCCAG
SEQ ID NO: 33
CTGGACTGTCGCTCCAG
SEQ ID NO: 34
CTGGACTGTTGCTCCAG
SEQ ID NO: 35
CTGGACTGTTCCTCCAG
SEQ ID NO: 36
CTGGATTGTCACTCCAG
SEQ ID NO: 37
CTGGATTGTCCCTCCAG
SEQ ID NO: 38
CTGGATTGTTTCTCCAG
SEQ ID NO: 39
CTGGATTGTTACTCCAG
SEQ ID NO: 40
CTGGATTGTCTCTCCAG
SEQ ID NO: 41
CTGGATTGTCGCTCCAG
SEQ ID NO: 42
CTGGATTGTTGCTCCAG
SEQ ID NO: 43
CTGGATTGTTCCTCCAG
SEQ ID NO: 44
CTGGACTGTCATTCCAG
SEQ ID NO: 45
CTGGACTGTCCTTCCAG
SEQ ID NO: 46
CTGGACTGTTTTTCCAG
SEQ ID NO: 47
CTGGACTGTTATTCCAG
SEQ ID NO: 48
CTGGACTGTCTTTCCAG
SEQ ID NO: 49
CTGGACTGTCGTTCCAG
SEQ ID NO: 50
CTGGACTGTTGTTCCAG
SEQ ID NO: 51
CTGGACTGTTCTTCCAG
SEQ ID NO: 52
CTTGCTTGTCCTGCAAG
SEQ ID NO: 53
CCTGCTTGTCCTGCAGG
SEQ ID NO: 54
AGCCCTTGTCCTGGGCT
SEQ ID NO: 55
CTCGGTTGTCCTCCGAG
SEQ ID NO: 56
CTGCCTTGTCCTGGCAG
SEQ ID NO: 57
ACGACTTGTCCTGTCGT
SEQ ID NO: 58
GCGGATTGTCCTTCCGT
SEQ ID NO: 59
GCGGATTGTCCTTCCGT
SEQ ID NO: 60
CCGGATTGTCCTTCCGG
SEQ ID NO: 61
CCGGTTTGTCCTACCGG
SEQ ID NO: 62
CCGCCTTGTCCTGGCGG
SEQ ID NO: 63
AGAGCTTGTCCTGCTCT
SEQ ID NO: 64
TCAGCTTGTCCTGCTGA
SEQ ID NO: 65
CGACCTTGTCCTGGTCG
SEQ ID NO: 66
CACCCTTGTCCTGGGTG
SEQ ID NO: 67
GTTGATTGTCCTTCAAC
SEQ ID NO: 68
CTAGCTTGTCCTGTTAG
SEQ ID NO: 69
CTACCTTGTCCTGGTAG
SEQ ID NO: 70
CCAGATTGTCCTTCTGG
SEQ ID NO: 71
AGGGATTGTCCTTCCCT
SEQ ID NO: 72
GTTGATTGTCCTTCAAT
SEQ ID NO: 73
GGCGATTGTCCTTCGCT
SEQ ID NO: 74
CATCATTGTCCTTGATG
SEQ ID NO: 75
TCGGGTTGTCCTCCCGA
SEQ ID NO: 76
GGGGATTGTCCTTCCCC
SEQ ID NO: 77
ACTGGTTGTCCTCCAGT
SEQ ID NO: 78
CCGTCTTGTCCTGTCGG
SEQ ID NO: 79
CTTCGTTGTCCTCGAGG
SEQ ID NO: 80
CCGGTTTGTCCTACCGG
SEQ ID NO: 81
CTCCCTTGTCCTGGGAG
SEQ ID NO: 82
CACGCTTGTCCTGTGTG
SEQ ID NO: 83
CACCCTTGTCCTGGGTG
SEQ ID NO: 84
TCGCATTGTCCTTGCGA
SEQ ID NO: 85
CGCATTTGTCCTATGCG
SEQ ID NO: 86
ACTGATTGTCCTTCAGT
SEQ ID NO: 87
CCGGTTTGTCCTACCGG
SEQ ID NO: 88
GCAGATTGTCCTTCTGC
SEQ ID NO: 89
CCACCTTGTCCTGGTGG
SEQ ID NO: 90
CCTCCTTGTCCTGGAGG
SEQ ID NO: 91
ACCATCATTTTCATATCCTCCACTGGATTG
TCCTTCCAGACCACCATCATTTGCAATGA
SEQ ID NO: 92
ACCATCATTTTCATATCCTCCAACCACCAT
CATTTGCAATGACTGGATTGTCCTTCCAG
SEQ ID NO: 93
CTGGATTGTCCTTCCAGACCATCATTTTCA
TATCCTCCAACCACCATCATTTGCAATGA
SEQ ID NO: 94
TTGTCCTTCCAGCACCC
SEQ ID NO: 95
GGGTGCTGGATTGTCCT
SEQ ID NO: 96
TTGTCCTTCCAG
SEQ ID NO: 97
CTGGATTGTCCT
SEQ ID NO 98:
GCAGACUGUAAAUCUGC
SEQ ID NO: 99
CTGGACTGTAAATCCAG
SEQ ID NO: 100
CTGGACTGTTAATCCAG
SEQ ID NO: 101
CTGGACTGTCACTCCAG
SEQ ID NO: 102
GGGAGCAGACYGYAAATCTGCTCCC
SEQ ID NO: 103
YYGYYYY
SEQ ID NO: 104
YTGTYYY
SEQ ID NO: 105
YTGTCCY
SEQ ID NO: 106
YTGTYCC
SEQ ID NO: 107
YUGUYYY
SEQ ID NO: 108
YUGTYYY
SEQ ID NO: 109
YTGUYYY

What is claimed is:

1. A method of modifying a DNA molecule, the method comprising contacting the DNA molecule with a tRNA-guanine transglycosylase (TGT) enzyme and a PreQ1 analog,
wherein the DNA molecule comprises a guanine nucleobase within a loop portion of a hairpin in the DNA molecule, and
wherein the PreQ1 analog has the formula: 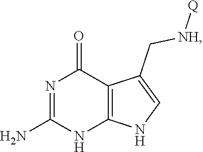
wherein Q is not hydrogen.

2. The method of claim 1, wherein the guanine nucleobase is in a YYGYYYY (SEQ ID NO: 103) sequence within the loop portion of the hairpin in the DNA molecule.

3. The method of claim 2, wherein the sequence within the loop portion of the hairpin is YTGTYYY (SEQ ID NO: 104), YTGTCCY (SEQ ID NO:105), YTGTYCC (SEQ ID NO:106), YUGUYYY (SEQ ID NO:107), YUGTYYY (SEQ ID NO:108), or YTGUYYY (SEQ ID NO:109).

4. The method of claim 3, wherein the sequence within the loop portion of the hairpin is YTGTCCY (SEQ ID NO:105) or YTGTYCC (SEQ ID NO:106).

5. The method of claim 1, wherein the DNA molecule is between about 15 nucleotides to about 30,000 nucleotides in length.

6. The method of claim 5, wherein the DNA molecule is between about 15 nucleotides to about 150 nucleotides in length.

7. The method of claim 1, wherein the DNA molecule is a gene, a cDNA, a non-coding DNA, an extracellular DNA (eDNA), an antisense oligonucleotide, a barcode, a probe, or a primer.

8. The method of claim 1, wherein the DNA molecule, TGT enzyme, and the PreQ1 analog are added to the reaction mixture simultaneously.

9. The method of claim 1, wherein the DNA molecule and the PreQ1 analog are added to the reaction mixture prior to adding the TGT enzyme.

10. The method of claim 1, wherein Q is -L¹-L²-R³;
L¹ and L² are independently a bond, —O—, —S—, —NH—, —C(O)NH₂, —NHC(O)—, —C(O)O—, —OC(O)—, —S(O)₂NH₂, —NHS(O)₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene, a peptide linker, or a cleavable linker;
R³ is a detectable moiety, a biomolecular moiety, a therapeutic moiety, hydrogen, halogen, —CX^{3.1}₃, —CHX^{3.1}₂, —CH₂X^{3.1}, —CN, —SOₙ₁R^{3A}, —SOᵥ₁NR^{3B}R^{3C}, —NHNR^{3B}R^{3C}, —ONR^{3B}R^{3C}, —NHC(O)NHNR^{3B}R^{3C}, —NHC(O)NR^{3B}R^{3C}, —N(O)ₘ₁, —NR^{3B}R^{3C}, —C(O)R^{3D}, —C(O)OR^{3D}, —C(O)NR^{3B}R^{3C}, —OR^{3A}, —NR^{3B}SO₂R^{3A}, —NR^{3B}C(O)R^{3D}, —NR^{3B}C(O)OR^{3D}, —NR^{3B}OR^{3D}, —OCX^{3.1}₃, —OCHX^{3.1}₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3A}, R^{3B}, R^{3C} and R^{3D} are independently hydrogen, halogen, —CF₃, —CCl₃, —CBr₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3B} and R^{3C} substituents bonded to the same nitrogen atom may optionally be joined to form a substituted or unsubstituted heterocycloalkyl or substituted or unsubstituted heteroaryl; and
X^{3.1} is independently —Cl, —Br, —I or —F.

11. The method of claim 10, wherein L¹ or L² comprises a photo-cleavable site.

12. The method of claim 10, wherein L¹ or L² comprises a peptide linker.

13. The method of claim 12, wherein said peptide linker comprises a protease cleavable site.

14. The method of claim 13, wherein said protease cleavable site is a matrix metalloprotease (MMP) cleavage site, a metalloprotease domain-containing (ADAM) metalloprotease cleavage site, a lysosomal cathepsin cleavage site (ALAL), or a legumain endopeptidase cleavage site (TANL).

15. The method of claim 10, wherein L¹ comprises a pH sensitive cleavable site.

16. The method of claim 10, wherein R³ is a detectable moiety.

17. The method of claim 16, wherein the detectable moiety is a fluorescent moiety.

18. A method of substituting a guanine nucleobase within a DNA molecule with a PreQ1 analog, the method comprising contacting the DNA molecule and PreQ1 analog with a tRNA-guanine transglycosylase (TGT) enzyme,
wherein the guanine nucleobase is within a loop portion of a hairpin in the DNA molecule, and
wherein the PreQ1 analog has the formula: 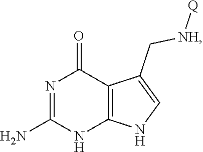
wherein Q is not hydrogen.

19. The method of claim 18, wherein the guanine nucleobase is in a YYGYYYY (SEQ ID NO: 103) sequence within the loop portion of the hairpin in the DNA molecule.

20. The method of claim 19, wherein the sequence within the loop portion of the hairpin is YTGTCCY (SEQ ID NO:105), YTGTYCC (SEQ ID NO: 106), YUGUYYY (SEQ ID NO:107), YUGTYYY (SEQ ID NO:108) or YTGUYYY (SEQ ID NO: 109).

21. The method of claim 20, wherein the sequence within the loop portion of the hairpin is YTGTCCY (SEQ ID NO:105) or YTGTYCC (SEQ ID NO:106).

22. The method of claim 18, wherein the DNA molecule is between about 15 nucleotides to about 30,000 nucleotides in length.

23. The method of claim 22, wherein the DNA molecule is between about 15 nucleotides to about 150 nucleotides in length.

24. The method of claim 18, wherein the DNA molecule is a gene, a cDNA, a non-coding DNA, an extracellular DNA (eDNA), an antisense oligonucleotide, a barcode, a probe, or a primer.

25. The method of claim 18, wherein the DNA molecule, TGT enzyme, and the PreQ1 analog are added to the reaction mixture simultaneously.

26. The method of claim 18, wherein the DNA molecule and the PreQ1 analog are added to the reaction mixture prior to adding the TGT enzyme.

27. The method of claim 18, wherein Q is -L¹-L²-R³;
L¹ and L² are independently a bond, —O—, —S—, —NH—, —C(O)NH₂, —NHC(O)—, —C(O)O—, —OC(O)—, —S(O)₂NH₂, —NHS(O)₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene, a peptide linker, or a cleavable linker;
R³ is a detectable moiety, a biomolecular moiety, a therapeutic moiety, hydrogen, halogen, —CX^{3.1}₃, —CHX^{3.1}₂, —CH₂X^{3.1}, —CN, —SOₙ₁R^{3A}, —SOᵥ₁NR^{3B}R^{3C}, —NHNR^{3B}R^{3C}, —ONR^{3B}R^{3C}, —NHC(O)NHNR^{3B}R^{3C}, —NHC(O)NR^{3B}R^{3C}, N(O)ₘ₁, —NR^{3B}R^{3C}, —C(O)R^{3D}, —C(O)OR^{3D}, —C(O)NR^{3B}R^{3C}, —OR^{3A}, —NR^{3B}SO₂R^{3A}, —NR^{3B}C(O)R^{3D}, —NR^{3B}C(O)OR^{3D}, —NR^{3B}OR^{3D}, —OCX^{3.1}₃, —OCHX^{3.1}₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3A}, R^{3B}, R^{3C} and R^{3D} are independently hydrogen, halogen, —CF₃, —CCl₃, —CBr₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3B} and R^{3C} substituents bonded to the same nitrogen atom may optionally be joined to form a substituted or unsubstituted heterocycloalkyl or substituted or unsubstituted heteroaryl; and
X^{3.1} is independently —Cl, —Br, —I or —F.

28. The method of claim 27, wherein L¹ or L² comprises a photo-cleavable site.

29. The method of claim 27, wherein L¹ or L² comprises a peptide linker.

30. The method of claim 29, wherein said peptide linker comprises a protease cleavable site.

31. The method of claim 30, wherein said protease cleavable site is a matrix metalloprotease (MMP) cleavage site, a metalloprotease domain-containing (ADAM) metalloprotease cleavage site, a lysosomal cathepsin cleavage site (ALAL), or a legumain endopeptidase cleavage site (TANL).

32. The method of claim 27, wherein L¹ comprises a pH sensitive cleavable site.

33. The method of any one of claims 27-32, wherein R³ is a detectable moiety.

34. The method of claim 33, wherein the detectable moiety is a fluorescent moiety.

35. A DNA compound having the formula: 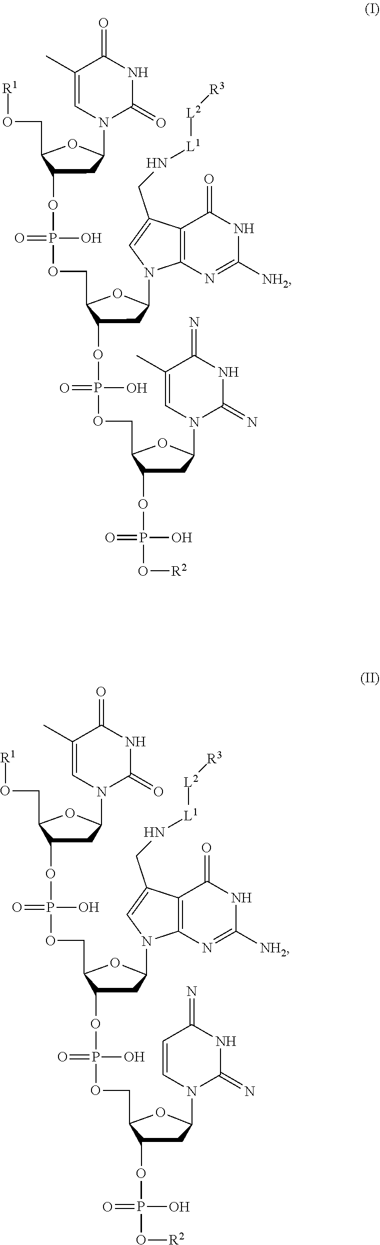 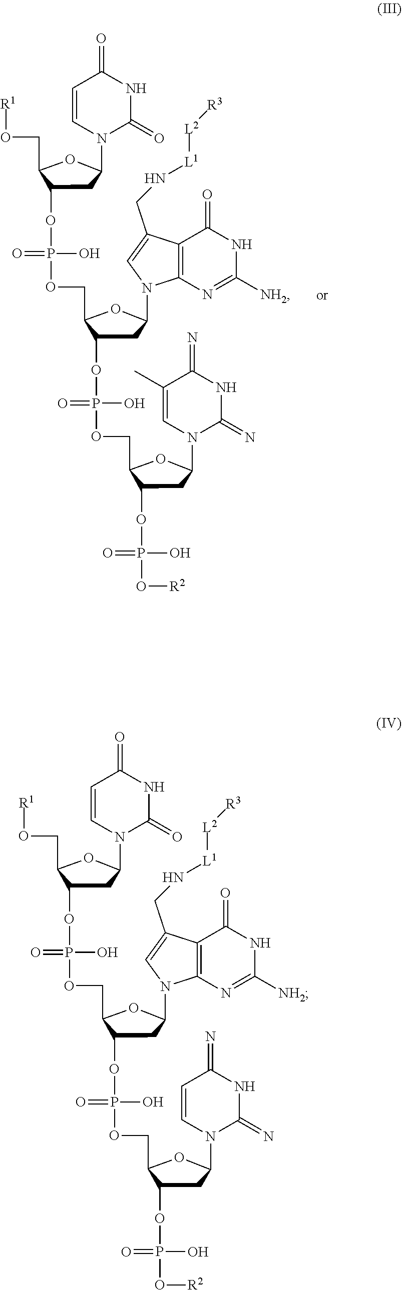
wherein
R¹ is hydrogen, a deoxynucleotide, or a first DNA sequence comprising the 5′ of a DNA hairpin;
R² is hydrogen, a deoxynucleotide, or a second DNA sequence comprising the 3′ of the DNA hairpin;
L¹ and L² are independently a bond, —O—, —S—, —NH—, —C(O)NH₂, —NHC(O)—, —C(O)O—, —OC(O)—, —S(O)₂NH₂, —NHS(O)₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene, a peptide linker, or a cleavable linker;
R³ is a detectable moiety, a biomolecular moiety, a therapeutic moiety, hydrogen, halogen, —CX^{3.1}₃, —CHX^{3.1}₂, —CH₂X^{3.1}, —CN, —SOₙ₁R^{3A}, —SOᵥ₁NR^{3B}R^{3C}, —NHNR^{3B}R^{3C}, —ONR^{3B}R^{3C}, —NHC(O)NHNR^{3B}R^{3C}, —NHC(O)NR^{3B}R^{3C}, —N(O)ₘ₁, —NR^{3B}R^{3C}, —C(O)R^{3D}, —C(O)OR^{3D}, —C(O)NR^{3B}R^{3C}, —OR^{3A}, —NR^{3B}SO₂R^{3A}, —NR^{3B}C(O)R^{3D}, —NR^{3B}C(O)OR^{3D}, —NR^{3B}OR^{3D}, —OCX^{3.1}₃, —OCHX^{3.1}₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3A}, R^{3B}, R^{3C} and R^{3D} are independently hydrogen, halogen, —CF₃, —CCl₃, —CBr₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂,
—NHC(O)NHNH₂, —NHC(O)NH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂,
—OCHI₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3B} and R^{3C} substituents bonded to the same nitrogen atom may optionally be joined to form a substituted or unsubstituted heterocycloalkyl or substituted or unsubstituted heteroaryl; and
X^{3.1} is independently —Cl, —Br, —I or —F.

36. The DNA compound of claim 35, having the formula:

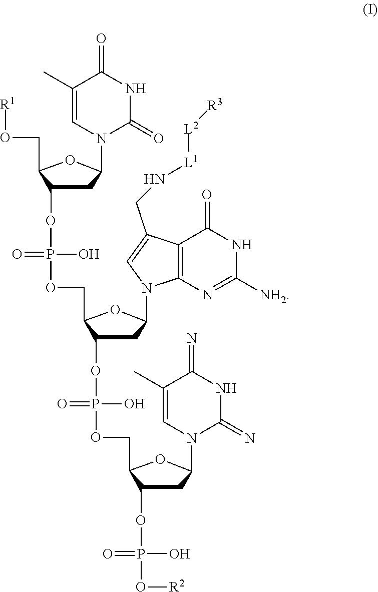

37. The DNA compound of claim 35, having the formula.

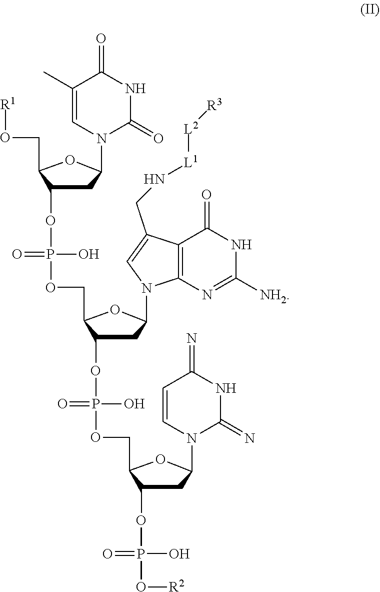

38. The DNA compound of claim 35, having the formula:

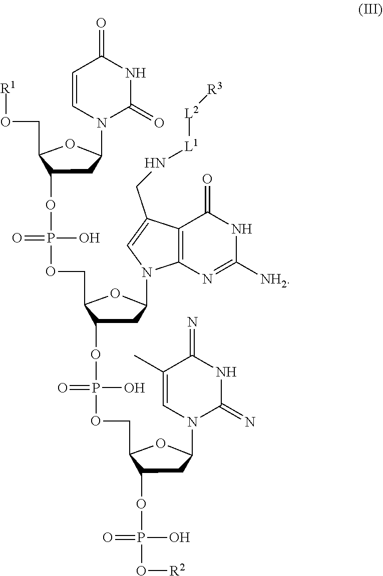

39. The DNA compound of claim 35, having the formula.

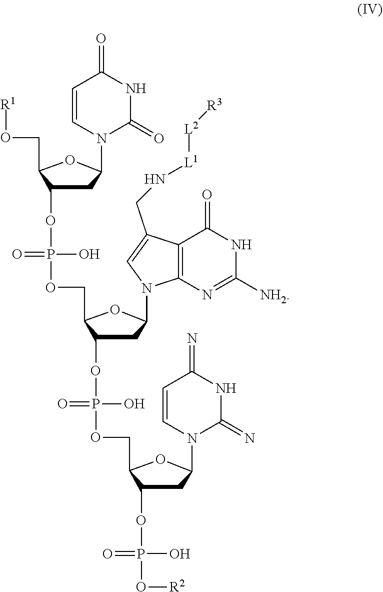

40. The DNA compound of claim 35, wherein R and R² are independently hydrogen.

41. The DNA compound of claim 35, wherein R¹ and R² are independently a deoxynucleotide.

42. The DNA compound of claim 41, wherein R¹ and R² are independently a deoxyadenosine, deoxyguanosine, thymidine, deoxyuridine, or deoxycytidine.

43. The DNA compound of claim 35, wherein R¹ and R² are independently a first DNA sequence comprising the 5′ of a DNA hairpin and a second DNA sequence comprising the 3′ of the DNA hairpin.

44. The DNA compound of claim 35, wherein L¹ is a bond or unsubstituted 2 to 12 membered heteroalkylene.

45. The DNA compound of claim 35, wherein L² is a bond or unsubstituted 2 to 12 membered heteroalkylene.

46. The DNA compound of claim 35, wherein L¹ or L² comprises a photo-cleavable site.

47. The DNA compound of claim 35, wherein L¹ or L² comprises a peptide linker.

48. The DNA method of claim 47, wherein said peptide linker comprises a protease cleavable site.

49. The DNA compound of claim 48, wherein said protease cleavable site is a matrix metalloprotease (MMP) cleavage site, a metalloprotease domain-containing (ADAM) metalloprotease cleavage site, a lysosomal cathepsin cleavage site (ALAL), or a legumain endopeptidase cleavage site (TANL).

50. The DNA compound of claim 35, wherein L¹ comprises a pH sensitive cleavable site.

51. The DNA compound of claim 35, wherein R³ is a detectable moiety.

52. The DNA compound of claim 51, wherein the detectable moiety is a fluorescent compound.

53. A cell comprising the DNA compound of claim 35.

54. The cell of claim 53, further comprising a tRNA-guanine transglycosylase (TGT) enzyme.
